(19) United States
(12) Patent Application Publication
(10) Pub. No.: US 2026/0308228 A1
(43) Pub. Date: Oct. 8, 2026

(54) MHC CLASS I MOLECULES

(71) Applicant: Immudex ApS, Virum (DK)

(72) Inventors: Thomas Holberg BLICHER, Copenhagen Ø (DK); Tobias Guldbech MORTENSEN, Copenhagen N (DK); Liselotte BRIX, Bagsværd (DK)

(21) Appl. No.: 19/482,841

(22) PCT Filed: May 10, 2024
(86) PCT No.: PCT/EP2024/062917

§ 371 Date: Nov. 10, 2025

(30) Foreign Application Priority Data
May 11, 2023 (EP) 23172833.8

(51) Int. Cl. C07K 14/705; A61K 38/00; C07K 19/00; C12N 15/63; G01N 33/50; G01N 33/68

(52) U.S. Cl. CPC C07K 14/70539; A61K 38/00; C07K 19/00; C12N 15/63; G01N 33/5091; G01N 33/5094; G01N 33/68; G01N 2333/70539; G01N 2800/00

(57) ABSTRACT

The present disclosure relates to MHC class I molecules, such as HLA-E, and peptide-MHC class I complexes with improved stability and/or refolding yield due to one or more introduced disulphide bridges. The disclosure further provides MHC class I multimers, cells, such as stem cells, comprising said MHC class I molecules, and various methods for their use.

### TECHNICAL FIELD

The present disclosure relates to MHC class I molecules and peptide-MHC class I complexes. In particular, it relates to MHC class I molecules and peptide-MHC class I complexes with improved stability and/or refolding yield that retain a native-like conformation to allow recognition by T cell receptors or other MHC class I complex-engaging receptors.

### BACKGROUND

MHC molecules present peptides of endogenous and exogenous origin to T cells of the immune system and thus play a central role in the surveillance of all cells in the body. The peptide is bound in a specialised binding groove in the MHC molecule comprised of two alpha-helices on top of a beta-sheet floor, and the groove is lined with binding pockets, which define the unique peptide specificity of each MHC molecule.

The classical MHC class I molecules in humans are known as class I human leukocyte antigens (HLA). These are distributed between three genetic loci, HLA-A, —B and —C, and serve general immune surveillance functions. In addition to these, a range of non-classical MHC molecules—also known as MHC-Ib (HLA-lb in humans)—serve more specialised functions. Among these is HLA-E (or its functional equivalent in non-human organisms), which binds to signal peptide fragments derived from other MHC molecules. This allows natural killer (NK) cells to monitor the general expression level of MHC molecules through HLA-E: Cells, which do not express MHC molecules also lack HLA-E and are eliminated by NK cells. This ensures that cells cannot hide from T cell immune surveillance by downregulating MHC expression.

In contrast to classical MHC class I molecules, which are encoded by some of the most highly polymorphic genes known, HLA-E exists almost exclusively in two, equally abundant allelic forms in the human population—HLA-E*01:01 and HLA-E*01:03—which differ by just a single amino acid residue in the extracellular domain. These two forms are equivalent in terms of peptide binding specificity and overall immunological role, and HLA-E can thus be considered functionally monomorphic from a technical perspective (Kanevskiy et al. 2019). While HLA-E typically binds to MHC signal peptide fragments, there is increasing evidence that HLA-E may also present peptide epitopes derived from bacteria, viruses and even cancers and thereby stimulate CD8+ T cells of the immune system (Sharpe et al., 2019; Yang et al., 2021). This ability to present pathogen- and cancer-derived peptides combined with its conserved functional and biological role makes HLA-E a highly attractive target for immunotherapy, as these properties bypass many of the challenges associated with targeting the highly polymorphic, classical MHC molecules.

Isolated peptide-MHC (pMHC) complexes are routinely used as tools in immunological research to detect specific subsets of T cells and other immune cells. While some pMHC complexes are readily produced or obtained from natural sources, other pMHC complexes are unstable or give low yields when produced recombinantly, either due to inherent instability or due to weak interactions with the bound ligand. In such cases, the MHC-Iigand complexes may become impractical to produce or use in technical or therapeutic applications.

In practical terms, the use of HLA-E complexes for the identification of HLA-α-reactive immune cells as well as for subsequent development of T-cell receptors (TCRs), antibodies or other HLA-E-targeting moieties is complicated by the fact that all known HLA-E complexes, unlike classical MHC molecules, are characterised by low stability, presumably due to the role of HLA-E as a rapid response system for monitoring general MHC expression.

While methods for the stabilisation of MHC complexes do exist, they are either not generally applicable (i.e. allele and/or peptide-dependent), technically complicated or may perturb the native conformation of the MHC complex, thus limiting their further use in technical or therapeutic applications. Some methods increase the stability of pMHC complexes by linking the peptide covalently to the MHC (Truscott et al., 2007; Mitaksov et al., 2007), while others utilise amino acid substitutions to stabilise the peptide-binding groove itself (e.g. US20140162293 A1). One method for stabilising HLA-E, which retains its native-like properties, uses covalent linking of the peptide through a disulphide bridge created between a cysteine-like, non-natural amino acid in the peptide and a cysteine residue introduced as a substitution of a residue in the HLA-E binding groove (WO2021001414 A1). Here, each peptide requires careful selection of the position of the non-natural amino acid within the peptide sequence as well as the anchoring position in the binding groove. Furthermore, since this method uses modified peptides and modifications in the peptide-facing residues of the binding groove, it is not applicable in in vivo (tissue transplants) or ex vivo settings, which rely on presentation of naturally processed peptide ligands.

In addition, refolding yields of MHC molecules are typically low, such as 10-20% in the best of cases.

There is thus a need for methods of stabilizing and/or improving the refolding yields of MHC class I molecules that are allele and/or peptide-independent, and which ensure that the MHC class I molecule retains its native-like binding and immune-mediating properties.

### SUMMARY

The present invention provides MHC class I molecules with improved stability and/or refolding yield, such as HLA-E molecules with improved stability and/or refolding yield, which exhibit native-like properties in terms of recognition by TCRs, NK cells, other MHC class I complex-engaging receptors and/or other immune cells, and thus is indistinguishable from wild type (WT) MHC class I molecules, such as WT HLA-E molecules with respect to these native recognition properties.

The present invention utilises a modified form the MHC class I molecule, where the introduction of one or more non-native disulphide bridges increases the stability of the resulting pMHC class I complexes. In addition, the stabilised form of the MHC class I molecule in some instances enables increased yields from recombinant production and refolding as compared to WT MHC class I molecules. The stabilising disulphide bridge may be placed on the underside of the peptide-binding groove, away from any of the residues involved in peptide binding or TCR recognition.

In some aspects the present disclosure provides an MHC class I molecule comprising or consisting of at least amino acids 1-51 and 91-137 of the amino acid sequence as set forth in SEQ ID NO: 1 or 2, or a variant of said MHC class I molecule having at least 85% sequence homology or identity thereto, such as at least 86% sequence homology or identity thereto, such as at least 87% sequence homology or identity thereto, such as at least 88% sequence homology or identity thereto, such as at least 89% sequence homology or identity thereto, such as at least 90% sequence homology or identity thereto, such as at least 91% sequence homology or identity thereto, such as at least 92% sequence homology or identity thereto, such as at least 93% sequence homology or identity thereto, such as at least 94% sequence homology or identity thereto, such as at least 95% sequence homology or identity thereto, such as at least 96% sequence homology or identity thereto, such as at least 97% sequence homology or identity thereto, such as at least 98% sequence homology or identity thereto, or such as at least 99% sequence homology or identity thereto,
with the proviso that the amino acids corresponding to positions
   i) 4 and 102 of SEQ ID NO: 1 or 2, and/or
   ii) 12 and 94 of SEQ ID NO: 1 or 2, and/or
   iii) 23 and 37 of SEQ ID NO: 1 or 2, and/or
   iv) 115 and 125 of SEQ ID NO: 1 or 2
have been mutated to cysteines and linked by a disulphide bridge.

In some aspects of the present disclosure is provided an MHC class I molecule comprising or consisting of at least amino acids 1-51 and 91-137 of the amino acid sequence as set forth in SEQ ID NO: 1 or 2, or a variant of said MHC class I molecule having at least 85% sequence homology or identity thereto, such as at least 90% sequence homology or identity thereto, such as at least 95% sequence homology or identity thereto, or such as at least 98% sequence homology or identity thereto,
with the proviso that the amino acids corresponding to positions
   i) 4 and 102 of SEQ ID NO: 1 or 2, and/or
   ii) 12 and 94 of SEQ ID NO: 1 or 2, and/or
   iii) 23 and 37 of SEQ ID NO: 1 or 2, and/or
   iv) 115 and 125 of SEQ ID NO: 1 or 2
have been mutated to cysteines and linked by a disulphide bridge, wherein
   i. the MHC class I molecule has increased stability, such as an increased melting temperature, such as increased stability compared to a corresponding wild type MHC class I molecule of identical sequence except not comprising said cysteine mutations linked by a disulphide bridge, and/or
   ii. the MHC class I molecule comprises a refolding test peptide in its peptide-binding groove and has an increased refolding yield compared to a corresponding wild type MHC class I molecule of identical sequence except not comprising said cysteine mutations linked by a disulphide bridge, said corresponding wild type MHC class I molecule also comprising said refolding test peptide in its peptide-binding groove.

In some aspects the present disclosure provides an MHC class I multimer or an MHC class I/peptide multimer comprising at least two MHC class I molecules as described herein.

In some aspects, the present disclosure provides a method of stabilising and/or increasing the refolding yield of an MHC class I molecule,
wherein said MHC class I comprises or consists of at least amino acids 1-51 and 91-137 of the amino acid sequence as set forth in SEQ ID NO: 1 or 2, or a variant thereof having at least 85% sequence homology or identity thereto, such as at least 90% sequence homology or identity thereto, such as at least 95% sequence homology or identity thereto, or such as at least 98% sequence homology or identity to SEQ ID NO: 1 or 2,
said method comprising the step of mutating the amino acids corresponding to positions
   i) 4 and 102 of SEQ ID NO: 1 or 2, and/or
   ii) 12 and 94 of SEQ ID NO: 1 or 2, and/or
   iii) 23 and 37 of SEQ ID NO: 1 or 2, and/or
   iv) 115 and 125 of SEQ ID NO: 1 or 2
      to cysteines and linking each of the cysteines of i) with a disulphide bridge, each of the cysteines of ii) with a disulphide bridge, each of the cysteines of iii) with a disulphide bridge and/or each of the cysteines of iv) with a disulphide bridge.

In some aspects, the present disclosure provides a method for isolation of one or more immune cells, such as antigen-specific T cells, CD8+ T cells, MAIT cells and/or NK cells, a method for detecting an immune cell response, such as an antigen-specific T cell response, a method for monitoring an immune response, or a method for diagnosing a disease, said method comprising the steps of:
a) providing a sample comprising a population of immune cells, such as T cells;
b) providing one or more MHC class I monomers, MHC class I/peptide monomers, MHC class I multimers and/or MHC class I/peptide multimers according to the present disclosure, or a composition comprising one or more of said MHC class I monomers, MHC class I/peptide monomers, MHC class I multimers and/or MHC class I/peptide multimers;
c) contacting said MHC class I monomers, MHC class I/peptide monomers, MHC class I multimers and/or MHC class I/peptide multimers, or said composition, with said sample; and
d) isolating said immune cells, such as T cells specific for said MHC class I monomers, MHC class I/peptide monomers, MHC class I multimers and/or MHC class I/peptide multimers, or composition.

In some aspects, the present disclosure provides an MHC class I monomer, an MHC class I/peptide monomer, an MHC class I multimer, or an MHC class I/peptide multimer, or a combination thereof, for use as a medicament.

In some aspects, the present disclosure provides an MHC class I monomer, an MHC class I/peptide monomer, an MHC class I multimer, or an MHC class I/peptide multimer, or a combination thereof, for use in a method of limiting or preventing NK cell cytotoxicity during or following tissue transplantation of a transplant tissue into a recipient, such as a human being.

In one aspect the present disclosure provides a method for limiting or preventing NK cell cytotoxicity during or following tissue transplantation of a transplant tissue into a recipient, said method comprising the steps of:
a) eliminating expression of native MHC molecules in the transplant tissue prior to transplantation of said transplant tissue into the recipient;
b) transplanting said transplant tissue into the recipient,

wherein the method further comprises the steps of:
c) introducing an MHC class I molecule according to the present disclosure into said transplant tissue prior to or after transplantation of said transplant tissue into the recipient of step b).

In some aspects the present disclosure provides a nucleic acid encoding the MHC class I monomers, MHC class I/peptide monomers, MHC class I multimers and/or MHC class I/peptide multimers.

In some aspects the present disclosure provides a vector comprising a nucleic acid encoding the MHC class I monomers, MHC class I/peptide monomers, MHC class I multimers and/or MHC class I/peptide multimers.

In some aspects the present disclosure provides a cell, such as a stem cell comprising an MHC class I monomer, or an MHC class I/peptide monomer.

### DESCRIPTION OF DRAWINGS

FIG. 1 shows histograms from the flow cytometry analysis of TCR Dextramer®-stained HLA-E beads for each HLA-E wild type. Beads carrying either inhA/HLA-E complexes or NY-ESO-1/HLA-A*02:01 complexes were tested for binding to fluorescent TCR Dextramer reagents carrying a TCR specific for either inhA/HLA-E or NY-ESO-1/HLA-A*02:01. High fluorescence intensity of the stained beads indicates specific binding by the TCR and thus confirms the native-like conformation of the corresponding HLA complex. No cross-binding between TCR Dextramer® reagents and HLA beads was observed for any of the HLA-E variants.

FIG. 2 shows stained peripheral blood mononuclear cells (PBMC) with UL40/HLA-E wild type Dextramer® reagent. A) Gating strategy: Selection of lymphocytes based on forward (FSC-H) and side (SSC-H) scatter profiles. Selection of single cells based on forward scatter signal area versus height. Live, CD4−/CD19− cells were subsequently selected and divided in four subsets based on their expression of CD8 and CD3. The four subsets contain 1) NKT cells, 2) CD8+ T cells, 3) double-negative T cells, and 4) NK cells. B) Comparing HLA-E Dextramer® staining of three cell subsets (NK, NKT, and CD8+ T cells) with their expression of the NK-cell-specific marker CD16. C) Like B) but using CD56, another NK-cell-specific marker. For each marker, the corresponding fluorochrome is given in parenthesis.

### DETAILED DESCRIPTION

### Definitions

As used herein, the singular forms “a”, “an” and “the” include plural referents unless the context clearly states otherwise. Thus, for example, reference to “MHC class I molecule” includes a plurality of MHC class I molecules.

As used herein, the term “variant” refers to either a naturally occurring variation of a given peptide or a recombinantly prepared variation of a given peptide or protein in which one or more amino acid residues have been modified by amino acid substitution, addition, or deletion.

The term “stability” as used herein refers to stability of the folded MHC class I molecule either with or without a peptide in the binding grove. In some embodiments, said stability may be measured by determining the melting temperature for the MHC class I molecule. Thus, an increased stability of an MHC class I molecule compared to a corresponding wild type MHC class I may be an increased melting temperature. The melting temperature of a molecule may be determined by any useful method known in the art, such as by nano differential scanning fluorimetry (nanoDSF). For example, the MHC class I molecule may be refolded with or without a peptide bound in the binding grove, and then subjected to thermal shift analysis through nanoscale differential scanning, e.g. by performing thermal denaturation by increasing the temperature from 20° C. to 95° C. at a rate of 1° C./minute, and determining the melting temperature for the MHC class I molecule from the first derivative of the 350 nm/330 nm ratio (d[F350/F330]/dT).

The term “refolding yield” as used herein refers to the actual yield of peptide-MHC complex (mass). Relative yield is obtained by comparing the actual yield with the theoretical yield, which is calculated by assuming that all MHC heavy chain (HC) added to a refolding mix forms complexes with peptide and light chain (β₂m). For example, the refolding yield may be calculated from the amount of denatured MHC HC vs amount of de novo-folded complete MHC molecules and correcting for difference in molar weight. Refolding yields can be measured by any method that allows for protein quantification, either by concentration or absolute amount: Absorption at 280 nm, amino acid analysis, mass spectrometry, Bradford analysis, bicinchoninic acid (BCA) analysis,—etc. These methods can thus be used to compare actual MHC peptide yield with the theoretical yield assuming that all added MHC HC forms complexes with peptide and β₂m and correcting for differences in molar mass of the HC versus a full complex.

The term “sequence homology” as used herein is used herein to refer to a comparison between amino acid sequences. As will be appreciated by those of ordinary skill in the art, two sequences are generally considered to be “homologous” if they contain homologous residues in corresponding positions. Homologous residues may be identical residues. Alternatively, homologous residues may be non-identical residues with appropriately similar structural and/or functional characteristics. For example, as is well known by those of ordinary skill in the art, certain amino acids are typically classified as “hydrophobic” or “hydrophilic” amino acids, and/or as having “polar” or “non-polar” side chains. Substitution of one amino acid for another of the same type may often be considered a “homologous” substitution. Typical amino acid categorizations are summarized in Table 1, below.

TABLE 1
Amino acid properties

| | | | | Side chain | |
|---|---|---|---|---|---|
| | 3- | 1- | Side chain | acidity or | Hydropathy |
| Amino Acid | Letter | Letter | polarity | basicity | index |
| Alanine | Ala | A | nonpolar | neutral | 1.8 |
| Arginine | Arg | R | polar | basic | −4.5 |
| Asparagine | Asn | N | polar | neutral | −3.5 |
| Aspartic acid | Asp | D | polar | acidic | −3.5 |
| Cysteine | Cys | C | polar | neutral | 2.5 |
| Glutamic acid | Glu | E | polar | acidic | −3.5 |
| Glutamine | Gln | Q | polar | neutral | −3.5 |
| Glycine | Gly | G | nonpolar | neutral | −0.4 |
| Histidine | His | H | polar | basic | −3.2 |
| Isoleucine | Ile | I | nonpolar | neutral | 4.5 |
| Leucine | Leu | L | nonpolar | neutral | 3.8 |
| Lysine | Lys | K | polar | basic | −3.9 |
| Methionine | Met | M | nonpolar | neutral | 1.9 |
| Phenylalanine | Phe | F | nonpolar | neutral | 2.8 |
| Proline | Pro | P | nonpolar | neutral | −1.6 |
| Serine | Ser | S | polar | neutral | −0.8 |
| Threonine | Thr | T | polar | neutral | −0.7 |
| Tryptophan | Trp | W | polar | neutral | −0.9 |
| Tyrosine | Tyr | Y | polar | neutral | −1.3 |
| Valine | Val | V | nonpolar | neutral | 4.2 |

As is well known in this art, amino acid or nucleic acid sequences may be compared using any of a variety of algorithms, including those available in commercial computer programs such as BLASTN for nucleotide sequences and BLASTP, gapped BLAST, and PSI-BLAST for amino acid sequences. Exemplary such programs are described in Altschul, et al., Basic local alignment search tool, J. Mol. Biol., 215(3): 403-410, 1990; Altschul, et al., Methods in Enzymology; Altschul, et al., “Gapped BLAST and PSI-BLAST: a new generation of protein database search programs”, Nucleic Acids Res. 25:3389-3402, 1997; Baxevanis, et al., Bioinformatics: A Practical Guide to the Analysis of Genes and Proteins, Wiley, 1998; and Misener, et al., (eds.), Bioinformatics Methods and Protocols (Methods in Molecular Biology, Vol. 132), Humana Press, 1999; all of the foregoing of which are incorporated herein by reference. In addition to identifying homologous sequences, the programs mentioned above typically provide an indication of the degree of homology, such as a percentage sequence homology.

The term “sequence identity” as used herein describes the relatedness between two amino acid sequences or between two nucleotide sequences, i.e. a candidate sequence (e.g. a mutant sequence) and a reference sequence (such as a wild type sequence) based on their pairwise alignment. For purposes of the present invention, the sequence identity between two amino acid sequences is determined using the Needleman-Wunsch algorithm (Needleman and Wunsch, 1970, J. Mo/. Biol. 48: 443-453) as implemented in the Needle program of the EMBOSS package (EMBOSS: The European Molecular Biology Open Software Suite, Rice et al., 2000, Trends Genet. 16: 276-277,), preferably version 5.0.0 or later (available at https://www.ebi.ac.uk/Tools/psa/emboss_needle/). The parameters used are gap open penalty of 10, gap extension penalty of 0.5, and the EBLOSUM62 (EMBOSS version of 30 BLOSUM62) substitution matrix. The output of Needle labeled “longest identity” (obtained using the -nobrief option) is used as the percent identity and is calculated as follows: (Identical Residues×100)/(Length of Alignment−Total Number of Gaps in Alignment). The Needleman-Wunsch algorithm is also used to determine whether a given amino acid in a sequence other than the reference sequence corresponds to a given position of the reference sequence. For purposes of the present invention, the sequence identity between two nucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the DNAFULL (EMBOSS version of NCBI NUC4.4) substitution matrix. The output of Needle labeled “longest identity” (obtained using the -nobrief option) is used as the percent identity and is calculated as follows: (Identical Deoxyribonucleotides×100)/(Length of Alignment—Total Number of Gaps in Alignment). Sequence identity is always measured compared to the full-length reference sequence, i.e. truncated proteins with no gaps or mismatches are not considered 100% sequence identical to the reference sequence.

The term “mutations” as used herein include insertions, deletions, or substitutions in the amino acid sequence of a protein. A mutant polypeptide or protein may be described as carrying a mutation, when it comprises an amino acid sequence differing from the wild type sequence.

### MHC Class I Molecules

The present disclosure relates to MHC class I molecules with increased stability and/or refolding yield compared to the corresponding wild type MHC class I molecules.

Thus, in some embodiments of the present disclosure, the MHC class I molecule according to the present disclosure comprises two cysteine mutations linked by a disulphide bridge which has increased stability and/or refolding yield compared to a corresponding wild type MHC class I molecule of identical sequence except not comprising said cysteine mutations linked by a disulphide bridge.

In some aspects the present disclosure provides an MHC class I molecule comprising or consisting of at least amino acids 1-51 and 91-137 of the amino acid sequence as set forth in SEQ ID NO: 1 or 2, or a variant of said MHC class I molecule having at least 85% sequence homology or identity thereto, such as at least 86% sequence homology or identity thereto, such as at least 87% sequence homology or identity thereto, such as at least 88% sequence homology or identity thereto, such as at least 89% sequence homology or identity thereto, such as at least 90% sequence homology or identity thereto, such as at least 91% sequence homology or identity thereto, such as at least 92% sequence homology or identity thereto, such as at least 93% sequence homology or identity thereto, such as at least 94% sequence homology or identity thereto, such as at least 95% sequence homology or identity thereto, such as at least 96% sequence homology or identity thereto, such as at least 97% sequence homology or identity thereto, such as at least 98% sequence homology or identity thereto, or such as at least 99% sequence homology or identity thereto,
with the proviso that the amino acids corresponding to positions
   i) 4 and 102 of SEQ ID NO: 1 or 2, and/or
   ii) 12 and 94 of SEQ ID NO: 1 or 2, and/or
   iii) 23 and 37 of SEQ ID NO: 1 or 2, and/or
   iv) 115 and 125 of SEQ ID NO: 1 or 2
have been mutated to cysteines and linked by a disulphide bridge.

In some embodiments the present disclosure provides an MHC class I molecule comprising or consisting of at least amino acids 1-137 of the amino acid sequence as set forth in SEQ ID NO: 1 or 2, or a variant of said MHC class I molecule having at least 85% sequence homology or identity thereto, such as at least 86% sequence homology or identity thereto, such as at least 87% sequence homology or identity thereto, such as at least 88% sequence homology or identity thereto, such as at least 89% sequence homology or identity thereto, such as at least 90% sequence homology or identity thereto, such as at least 91% sequence homology or identity thereto, such as at least 92% sequence homology or identity thereto, such as at least 93% sequence homology or identity thereto, such as at least 94% sequence homology or identity thereto, such as at least 95% sequence homology or identity thereto, such as at least 96% sequence homology or identity thereto, such as at least 97% sequence homology or identity thereto, such as at least 98% sequence homology or identity thereto, or such as at least 99% sequence homology or identity thereto,
with the proviso that the amino acids corresponding to positions
   i) 4 and 102 of SEQ ID NO: 1 or 2, and/or
   ii) 12 and 94 of SEQ ID NO: 1 or 2, and/or
   iii) 23 and 37 of SEQ ID NO: 1 or 2, and/or
   iv) 115 and 125 of SEQ ID NO: 1 or 2
have been mutated to cysteines and linked by a disulphide bridge.

In some embodiments of the present disclosure, the MHC class I molecule comprises or consists of at least amino acids 1-180 of the amino acid sequence according to SEQ ID NO: 1 or 2, or a variant of said MHC class I molecule having at least 85% sequence homology or identity thereto, such as at least 86% sequence homology or identity thereto, such as at least 87% sequence homology or identity thereto, such as at least 88% sequence homology or identity thereto, such as at least 89% sequence homology or identity thereto, such as at least 90% sequence homology or identity thereto, such as at least 91% sequence homology or identity thereto, such as at least 92% sequence homology or identity thereto, such as at least 93% sequence homology or identity thereto, such as at least 94% sequence homology or identity thereto, such as at least 95% sequence homology or identity thereto, such as at least 96% sequence homology or identity thereto, such as at least 97% sequence homology or identity thereto, such as at least 98% sequence homology or identity thereto, or such as at least 99% sequence homology or identity thereto,
with the proviso that the amino acids corresponding to positions
   i) 4 and 102 of SEQ ID NO: 1 or 2, and/or
   ii) 12 and 94 of SEQ ID NO: 1 or 2, and/or
   iii) 23 and 37 of SEQ ID NO: 1 or 2, and/or
   iv) 115 and 125 of SEQ ID NO: 1 or 2
have been mutated to cysteines and linked by a disulphide bridge.

In some embodiments of the present disclosure, the MHC class I molecule comprises or consists of at least amino acids 1-37 of the amino acid sequence according to SEQ ID NO: 1 or 2, such as at least amino acids 1-45 of the amino acid sequence according to SEQ ID NO: 1 or 2, such as at least amino acids 1-60 of the amino acid sequence according to SEQ ID NO: 1 or 2, such as at least amino acids 1-80 of the amino acid sequence according to SEQ ID NO: 1 or 2, such as at least amino acids 1-100 of the amino acid sequence according to SEQ ID NO: 1 or 2, such as at least amino acids 1-120 of the amino acid sequence according to SEQ ID NO: 1 or 2, such as at least amino acids 1-140 of the amino acid sequence according to SEQ ID NO: 1 or 2, such as at least amino acids 1-160 of the amino acid sequence according to SEQ ID NO: 1 or 2, such as at least amino acids 1-180 of the amino acid sequence according to SEQ ID NO: 1 or 2, such as at least amino acids 1-200 of the amino acid sequence according to SEQ ID NO: 1 or 2, such as at least amino acids 1-220 of the amino acid sequence according to SEQ ID NO: 1 or 2, such as at least amino acids 1-240 of the amino acid sequence according to SEQ ID NO: 1 or 2, such as at least amino acids 1-260 of the amino acid sequence according to SEQ ID NO: 1 or 2, such as at least amino acids 1-276 of the amino acid sequence according to SEQ ID NO: 1 or 2, or a variant of said MHC class I molecule having at least 85% sequence homology or identity thereto, such as at least 86% sequence homology or identity thereto, such as at least 87% sequence homology or identity thereto, such as at least 88% sequence homology or identity thereto, such as at least 89% sequence homology or identity thereto, such as at least 90% sequence homology or identity thereto, such as at least 91% sequence homology or identity thereto, such as at least 92% sequence homology or identity thereto, such as at least 93% sequence homology or identity thereto, such as at least 94% sequence homology or identity thereto, such as at least 95% sequence homology or identity thereto, such as at least 96% sequence homology or identity thereto, such as at least 97% sequence homology or identity thereto, such as at least 98% sequence homology or identity thereto, or such as at least 99% sequence homology or identity thereto,
with the proviso that the amino acids corresponding to positions
   i) 4 and 102 of SEQ ID NO: 1 or 2, and/or
   ii) 12 and 94 of SEQ ID NO: 1 or 2, and/or
   iii) 23 and 37 of SEQ ID NO: 1 or 2, and/or
   iv) 115 and 125 of SEQ ID NO: 1 or 2
have been mutated to cysteines and linked by a disulphide bridge.

In some embodiments of the present disclosure, the MHC class I molecule comprises or consists of at least amino acids 1-102 of the amino acid sequence according to SEQ ID NO: 1 or 2, such as at least amino acids 1-120 of the amino acid sequence according to SEQ ID NO: 1 or 2, such as at least amino acids 1-140 of the amino acid sequence according to SEQ ID NO: 1 or 2, such as at least amino acids 1-160 of the amino acid sequence according to SEQ ID NO: 1 or 2, such as at least amino acids 1-180 of the amino acid sequence according to SEQ ID NO: 1 or 2, such as at least amino acids 1-200 of the amino acid sequence according to SEQ ID NO: 1 or 2, such as at least amino acids 1-220 of the amino acid sequence according to SEQ ID NO: 1 or 2, such as at least amino acids 1-240 of the amino acid sequence according to SEQ ID NO: 1 or 2, such as at least amino acids 1-260 of the amino acid sequence according to SEQ ID NO: 1 or 2, such as at least amino acids 1-276 of the amino acid sequence according to SEQ ID NO: 1 or 2, or a variant of said MHC class I molecule having at least 85% sequence homology or identity thereto, such as at least 86% sequence homology or identity thereto, such as at least 87% sequence homology or identity thereto, such as at least 88% sequence homology or identity thereto, such as at least 89% sequence homology or identity thereto, such as at least 90% sequence homology or identity thereto, such as at least 91% sequence homology or identity thereto, such as at least 92% sequence homology or identity thereto, such as at least 93% sequence homology or identity thereto, such as at least 94% sequence homology or identity thereto, such as at least 95% sequence homology or identity thereto, such as at least 96% sequence homology or identity thereto, such as at least 97% sequence homology or identity thereto, such as at least 98% sequence homology or identity thereto, or such as at least 99% sequence homology or identity thereto,
with the proviso that the amino acids corresponding to positions
   i) 4 and 102 of SEQ ID NO: 1 or 2,
have been mutated to cysteines and linked by a disulphide bridge.

In some embodiments of the present disclosure, the MHC class I molecule comprises or consists of at least amino acids 1-94 of the amino acid sequence according to SEQ ID NO: 1 or 2, such as at least amino acids 1-100 of the amino acid sequence according to SEQ ID NO: 1 or 2, such as at least amino acids 1-120 of the amino acid sequence according to SEQ ID NO: 1 or 2, such as at least amino acids 1-140 of the amino acid sequence according to SEQ ID NO: 1 or 2, such as at least amino acids 1-160 of the amino acid sequence according to SEQ ID NO: 1 or 2, such as at least amino acids 1-180 of the amino acid sequence according to SEQ ID NO: 1 or 2, such as at least amino acids 1-200 of the amino acid sequence according to SEQ ID NO: 1 or 2, such as at least amino acids 1-220 of the amino acid sequence according to SEQ ID NO: 1 or 2, such as at least amino acids 1-240 of the amino acid sequence according to SEQ ID NO: 1 or 2, such as at least amino acids 1-260 of the amino acid sequence according to SEQ ID NO: 1 or 2, such as at least amino acids 1-276 of the amino acid sequence according to SEQ ID NO: 1 or 2, or a variant of said MHC class I molecule having at least 85% sequence homology or identity thereto, such as at least 86% sequence homology or identity thereto, such as at least 87% sequence homology or identity thereto, such as at least 88% sequence homology or identity thereto, such as at least 89% sequence homology or identity thereto, such as at least 90% sequence homology or identity thereto, such as at least 91% sequence homology or identity thereto, such as at least 92% sequence homology or identity thereto, such as at least 93% sequence homology or identity thereto, such as at least 94% sequence homology or identity thereto, such as at least 95% sequence homology or identity thereto, such as at least 96% sequence homology or identity thereto, such as at least 97% sequence homology or identity thereto, such as at least 98% sequence homology or identity thereto, or such as at least 99% sequence homology or identity thereto,
with the proviso that the amino acids corresponding to positions
   ii) 12 and 94 of SEQ ID NO: 1 or 2,
have been mutated to cysteines and linked by a disulphide bridge.

In some embodiments of the present disclosure, the MHC class I molecule comprises or consists of at least amino acids 1-37 of the amino acid sequence according to SEQ ID NO: 1 or 2, such as at least amino acids 1-45 of the amino acid sequence according to SEQ ID NO: 1 or 2, such as at least amino acids 1-60 of the amino acid sequence according to SEQ ID NO: 1 or 2, such as at least amino acids 1-80 of the amino acid sequence according to SEQ ID NO: 1 or 2, such as at least amino acids 1-100 of the amino acid sequence according to SEQ ID NO: 1 or 2, such as at least amino acids 1-120 of the amino acid sequence according to SEQ ID NO: 1 or 2, such as at least amino acids 1-140 of the amino acid sequence according to SEQ ID NO: 1 or 2, such as at least amino acids 1-160 of the amino acid sequence according to SEQ ID NO: 1 or 2, such as at least amino acids 1-180 of the amino acid sequence according to SEQ ID NO: 1 or 2, such as at least amino acids 1-200 of the amino acid sequence according to SEQ ID NO: 1 or 2, such as at least amino acids 1-220 of the amino acid sequence according to SEQ ID NO: 1 or 2, such as at least amino acids 1-240 of the amino acid sequence according to SEQ ID NO: 1 or 2, such as at least amino acids 1-260 of the amino acid sequence according to SEQ ID NO: 1 or 2, such as at least amino acids 1-276 of the amino acid sequence according to SEQ ID NO: 1 or 2, or a variant of said MHC class I molecule having at least 85% sequence homology or identity thereto, such as at least 86% sequence homology or identity thereto, such as at least 87% sequence homology or identity thereto, such as at least 88% sequence homology or identity thereto, such as at least 89% sequence homology or identity thereto, such as at least 90% sequence homology or identity thereto, such as at least 91% sequence homology or identity thereto, such as at least 92% sequence homology or identity thereto, such as at least 93% sequence homology or identity thereto, such as at least 94% sequence homology or identity thereto, such as at least 95% sequence homology or identity thereto, such as at least 96% sequence homology or identity thereto, such as at least 97% sequence homology or identity thereto, such as at least 98% sequence homology or identity thereto, or such as at least 99% sequence homology or identity thereto,
with the proviso that the amino acids corresponding to positions
   iii) 23 and 37 of SEQ ID NO: 1 or 2,
have been mutated to cysteines and linked by a disulphide bridge.

In some embodiments of the present disclosure, the MHC class I molecule comprises or consists of at least amino acids 1-115 of the amino acid sequence according to SEQ ID NO: 1 or 2, such as at least amino acids 1-120 of the amino acid sequence according to SEQ ID NO: 1 or 2, such as at least amino acids 1-140 of the amino acid sequence according to SEQ ID NO: 1 or 2, such as at least amino acids 1-160 of the amino acid sequence according to SEQ ID NO: 1 or 2, such as at least amino acids 1-180 of the amino acid sequence according to SEQ ID NO: 1 or 2, such as at least amino acids 1-200 of the amino acid sequence according to SEQ ID NO: 1 or 2, such as at least amino acids 1-220 of the amino acid sequence according to SEQ ID NO: 1 or 2, such as at least amino acids 1-240 of the amino acid sequence according to SEQ ID NO: 1 or 2, such as at least amino acids 1-260 of the amino acid sequence according to SEQ ID NO: 1 or 2, such as at least amino acids 1-276 of the amino acid sequence according to SEQ ID NO: 1 or 2, or a variant of said MHC class I molecule having at least 85% sequence homology or identity thereto, such as at least 86% sequence homology or identity thereto, such as at least 87% sequence homology or identity thereto, such as at least 88% sequence homology or identity thereto, such as at least 89% sequence homology or identity thereto, such as at least 90% sequence homology or identity thereto, such as at least 91% sequence homology or identity thereto, such as at least 92% sequence homology or identity thereto, such as at least 93% sequence homology or identity thereto, such as at least 94% sequence homology or identity thereto, such as at least 95% sequence homology or identity thereto, such as at least 96% sequence homology or identity thereto, such as at least 97% sequence homology or identity thereto, such as at least 98% sequence homology or identity thereto, or such as at least 99% sequence homology or identity thereto,
with the proviso that the amino acids corresponding to positions
   iv) 115 and 125 of SEQ ID NO: 1 or 2
have been mutated to cysteines and linked by a disulphide bridge.

In some embodiments of the present disclosure, the MHC class I molecule comprises or consists of at least amino acids 1-102 of the amino acid sequence according to SEQ ID NO: 1 or 2, such as at least amino acids 1-120 of the amino acid sequence according to SEQ ID NO: 1 or 2, such as at least amino acids 1-140 of the amino acid sequence according to SEQ ID NO: 1 or 2, such as at least amino acids 1-160 of the amino acid sequence according to SEQ ID NO: 1 or 2, such as at least amino acids 1-180 of the amino acid sequence according to SEQ ID NO: 1 or 2, such as at least amino acids 1-200 of the amino acid sequence according to SEQ ID NO: 1 or 2, such as at least amino acids 1-220 of the amino acid sequence according to SEQ ID NO: 1 or 2, such as at least amino acids 1-240 of the amino acid sequence according to SEQ ID NO: 1 or 2, such as at least amino acids 1-260 of the amino acid sequence according to SEQ ID NO: 1 or 2, such as at least amino acids 1-276 of the amino acid sequence according to SEQ ID NO: 1 or 2, or a variant of said MHC class I molecule having at least 85% sequence homology or identity thereto, such as at least 86% sequence homology or identity thereto, such as at least 87% sequence homology or identity thereto, such as at least 88% sequence homology or identity thereto, such as at least 89% sequence homology or identity thereto, such as at least 90% sequence homology or identity thereto, such as at least 91% sequence homology or identity thereto, such as at least 92% sequence homology or identity thereto, such as at least 93% sequence homology or identity thereto, such as at least 94% sequence homology or identity thereto, such as at least 95% sequence homology or identity thereto, such as at least 96% sequence homology or identity thereto, such as at least 97% sequence homology or identity thereto, such as at least 98% sequence homology or identity thereto, or such as at least 99% sequence homology or identity thereto,
with the proviso that the amino acids corresponding to positions
   i) 4 and 102 of SEQ ID NO: 1 or 2, and
   ii) 12 and 94 of SEQ ID NO: 1 or 2,
have been mutated to cysteines and linked by a disulphide bridge.

In some embodiments of the present disclosure, the MHC class I molecule comprises or consists of at least amino acids 1-102 of the amino acid sequence according to SEQ ID NO: 1 or 2, such as at least amino acids 1-120 of the amino acid sequence according to SEQ ID NO: 1 or 2, such as at least amino acids 1-140 of the amino acid sequence according to SEQ ID NO: 1 or 2, such as at least amino acids 1-160 of the amino acid sequence according to SEQ ID NO: 1 or 2, such as at least amino acids 1-180 of the amino acid sequence according to SEQ ID NO: 1 or 2, such as at least amino acids 1-200 of the amino acid sequence according to SEQ ID NO: 1 or 2, such as at least amino acids 1-220 of the amino acid sequence according to SEQ ID NO: 1 or 2, such as at least amino acids 1-240 of the amino acid sequence according to SEQ ID NO: 1 or 2, such as at least amino acids 1-260 of the amino acid sequence according to SEQ ID NO: 1 or 2, such as at least amino acids 1-276 of the amino acid sequence according to SEQ ID NO: 1 or 2, or a variant of said MHC class I molecule having at least 85% sequence homology or identity thereto, such as at least 86% sequence homology or identity thereto, such as at least 87% sequence homology or identity thereto, such as at least 88% sequence homology or identity thereto, such as at least 89% sequence homology or identity thereto, such as at least 90% sequence homology or identity thereto, such as at least 91% sequence homology or identity thereto, such as at least 92% sequence homology or identity thereto, such as at least 93% sequence homology or identity thereto, such as at least 94% sequence homology or identity thereto, such as at least 95% sequence homology or identity thereto, such as at least 96% sequence homology or identity thereto, such as at least 97% sequence homology or identity thereto, such as at least 98% sequence homology or identity thereto, or such as at least 99% sequence homology or identity thereto,
with the proviso that the amino acids corresponding to positions
   i) 4 and 102 of SEQ ID NO: 1 or 2, and
   iii) 23 and 37 of SEQ ID NO: 1 or 2,
have been mutated to cysteines and linked by a disulphide bridge.

In some embodiments of the present disclosure, the MHC class I molecule comprises or consists of at least amino acids 1-125 of the amino acid sequence according to SEQ ID NO: 1 or 2, such as at least amino acids 1-140 of the amino acid sequence according to SEQ ID NO: 1 or 2, such as at least amino acids 1-160 of the amino acid sequence according to SEQ ID NO: 1 or 2, such as at least amino acids 1-180 of the amino acid sequence according to SEQ ID NO: 1 or 2, such as at least amino acids 1-200 of the amino acid sequence according to SEQ ID NO: 1 or 2, such as at least amino acids 1-220 of the amino acid sequence according to SEQ ID NO: 1 or 2, such as at least amino acids 1-240 of the amino acid sequence according to SEQ ID NO: 1 or 2, such as at least amino acids 1-260 of the amino acid sequence according to SEQ ID NO: 1 or 2, such as at least amino acids 1-276 of the amino acid sequence according to SEQ ID NO: 1 or 2, or a variant of said MHC class I molecule having at least 85% sequence homology or identity thereto, such as at least 86% sequence homology or identity thereto, such as at least 87% sequence homology or identity thereto, such as at least 88% sequence homology or identity thereto, such as at least 89% sequence homology or identity thereto, such as at least 90% sequence homology or identity thereto, such as at least 91% sequence homology or identity thereto, such as at least 92% sequence homology or identity thereto, such as at least 93% sequence homology or identity thereto, such as at least 94% sequence homology or identity thereto, such as at least 95% sequence homology or identity thereto, such as at least 96% sequence homology or identity thereto, such as at least 97% sequence homology or identity thereto, such as at least 98% sequence homology or identity thereto, or such as at least 99% sequence homology or identity thereto,
with the proviso that the amino acids corresponding to positions
   i) 4 and 102 of SEQ ID NO: 1 or 2, and
   iv) 115 and 125 of SEQ ID NO: 1 or 2
have been mutated to cysteines and linked by a disulphide bridge.

In some embodiments of the present disclosure, the MHC class I molecule comprises or consists of at least amino acids 1-94 of the amino acid sequence according to SEQ ID NO: 1 or 2, such as at least amino acids 1-100 of the amino acid sequence according to SEQ ID NO: 1 or 2, such as at least amino acids 1-120 of the amino acid sequence according to SEQ ID NO: 1 or 2, such as at least amino acids 1-140 of the amino acid sequence according to SEQ ID NO: 1 or 2, such as at least amino acids 1-160 of the amino acid sequence according to SEQ ID NO: 1 or 2, such as at least amino acids 1-180 of the amino acid sequence according to SEQ ID NO: 1 or 2, such as at least amino acids 1-200 of the amino acid sequence according to SEQ ID NO: 1 or 2, such as at least amino acids 1-220 of the amino acid sequence according to SEQ ID NO: 1 or 2, such as at least amino acids 1-240 of the amino acid sequence according to SEQ ID NO: 1 or 2, such as at least amino acids 1-260 of the amino acid sequence according to SEQ ID NO: 1 or 2, such as at least amino acids 1-276 of the amino acid sequence according to SEQ ID NO: 1 or 2, or a variant of said MHC class I molecule having at least 85% sequence homology or identity thereto, such as at least 86% sequence homology or identity thereto, such as at least 87% sequence homology or identity thereto, such as at least 88% sequence homology or identity thereto, such as at least 89% sequence homology or identity thereto, such as at least 90% sequence homology or identity thereto, such as at least 91% sequence homology or identity thereto, such as at least 92% sequence homology or identity thereto, such as at least 93% sequence homology or identity thereto, such as at least 94% sequence homology or identity thereto, such as at least 95% sequence homology or identity thereto, such as at least 96% sequence homology or identity thereto, such as at least 97% sequence homology or identity thereto, such as at least 98% sequence homology or identity thereto, or such as at least 99% sequence homology or identity thereto,
with the proviso that the amino acids corresponding to positions
   ii) 12 and 94 of SEQ ID NO: 1 or 2, and
   iii) 23 and 37 of SEQ ID NO: 1 or 2,
have been mutated to cysteines and linked by a disulphide bridge.

In some embodiments of the present disclosure, the MHC class I molecule comprises or consists of at least amino acids 1-125 of the amino acid sequence according to SEQ ID NO: 1 or 2, such as at least amino acids 1-140 of the amino acid sequence according to SEQ ID NO: 1 or 2, such as at least amino acids 1-160 of the amino acid sequence according to SEQ ID NO: 1 or 2, such as at least amino acids 1-180 of the amino acid sequence according to SEQ ID NO: 1 or 2, such as at least amino acids 1-200 of the amino acid sequence according to SEQ ID NO: 1 or 2, such as at least amino acids 1-220 of the amino acid sequence according to SEQ ID NO: 1 or 2, such as at least amino acids 1-240 of the amino acid sequence according to SEQ ID NO: 1 or 2, such as at least amino acids 1-260 of the amino acid sequence according to SEQ ID NO: 1 or 2, such as at least amino acids 1-276 of the amino acid sequence according to SEQ ID NO: 1 or 2, or a variant of said MHC class I molecule having at least 85% sequence homology or identity thereto, such as at least 86% sequence homology or identity thereto, such as at least 87% sequence homology or identity thereto, such as at least 88% sequence homology or identity thereto, such as at least 89% sequence homology or identity thereto, such as at least 90% sequence homology or identity thereto, such as at least 91% sequence homology or identity thereto, such as at least 92% sequence homology or identity thereto, such as at least 93% sequence homology or identity thereto, such as at least 94% sequence homology or identity thereto, such as at least 95% sequence homology or identity thereto, such as at least 96% sequence homology or identity thereto, such as at least 97% sequence homology or identity thereto, such as at least 98% sequence homology or identity thereto, or such as at least 99% sequence homology or identity thereto,
with the proviso that the amino acids corresponding to positions
   ii) 12 and 94 of SEQ ID NO: 1 or 2,
   iv) 115 and 125 of SEQ ID NO: 1 or 2
have been mutated to cysteines and linked by a disulphide bridge.

In some embodiments of the present disclosure, the MHC class I molecule comprises or consists of at least amino acids 1-125 of the amino acid sequence according to SEQ ID NO: 1 or 2, such as at least amino acids 1-140 of the amino acid sequence according to SEQ ID NO: 1 or 2, such as at least amino acids 1-160 of the amino acid sequence according to SEQ ID NO: 1 or 2, such as at least amino acids 1-180 of the amino acid sequence according to SEQ ID NO: 1 or 2, such as at least amino acids 1-200 of the amino acid sequence according to SEQ ID NO: 1 or 2, such as at least amino acids 1-220 of the amino acid sequence according to SEQ ID NO: 1 or 2, such as at least amino acids 1-240 of the amino acid sequence according to SEQ ID NO: 1 or 2, such as at least amino acids 1-260 of the amino acid sequence according to SEQ ID NO: 1 or 2, such as at least amino acids 1-276 of the amino acid sequence according to SEQ ID NO: 1 or 2, or a variant of said MHC class I molecule having at least 85% sequence homology or identity thereto, such as at least 86% sequence homology or identity thereto, such as at least 87% sequence homology or identity thereto, such as at least 88% sequence homology or identity thereto, such as at least 89% sequence homology or identity thereto, such as at least 90% sequence homology or identity thereto, such as at least 91% sequence homology or identity thereto, such as at least 92% sequence homology or identity thereto, such as at least 93% sequence homology or identity thereto, such as at least 94% sequence homology or identity thereto, such as at least 95% sequence homology or identity thereto, such as at least 96% sequence homology or identity thereto, such as at least 97% sequence homology or identity thereto, such as at least 98% sequence homology or identity thereto, or such as at least 99% sequence homology or identity thereto,
with the proviso that the amino acids corresponding to positions
   iii) 23 and 37 of SEQ ID NO: 1 or 2,
   iv) 115 and 125 of SEQ ID NO: 1 or 2
have been mutated to cysteines and linked by a disulphide bridge.

In some embodiments of the present disclosure, the MHC class I molecule comprises or consists of at least amino acids 1-102 of the amino acid sequence according to SEQ ID NO: 1 or 2, such as at least amino acids 1-120 of the amino acid sequence according to SEQ ID NO: 1 or 2, such as at least amino acids 1-140 of the amino acid sequence according to SEQ ID NO: 1 or 2, such as at least amino acids 1-160 of the amino acid sequence according to SEQ ID NO: 1 or 2, such as at least amino acids 1-180 of the amino acid sequence according to SEQ ID NO: 1 or 2, such as at least amino acids 1-200 of the amino acid sequence according to SEQ ID NO: 1 or 2, such as at least amino acids 1-220 of the amino acid sequence according to SEQ ID NO: 1 or 2, such as at least amino acids 1-240 of the amino acid sequence according to SEQ ID NO: 1 or 2, such as at least amino acids 1-260 of the amino acid sequence according to SEQ ID NO: 1 or 2, such as at least amino acids 1-276 of the amino acid sequence according to SEQ ID NO: 1 or 2, or a variant of said MHC class I molecule having at least 85% sequence homology or identity thereto, such as at least 86% sequence homology or identity thereto, such as at least 87% sequence homology or identity thereto, such as at least 88% sequence homology or identity thereto, such as at least 89% sequence homology or identity thereto, such as at least 90% sequence homology or identity thereto, such as at least 91% sequence homology or identity thereto, such as at least 92% sequence homology or identity thereto, such as at least 93% sequence homology or identity thereto, such as at least 94% sequence homology or identity thereto, such as at least 95% sequence homology or identity thereto, such as at least 96% sequence homology or identity thereto, such as at least 97% sequence homology or identity thereto, such as at least 98% sequence homology or identity thereto, or such as at least 99% sequence homology or identity thereto,
with the proviso that the amino acids corresponding to positions
   i) 4 and 102 of SEQ ID NO: 1 or 2, and
   ii) 12 and 94 of SEQ ID NO: 1 or 2, and
   iii) 23 and 37 of SEQ ID NO: 1 or 2,
have been mutated to cysteines and linked by a disulphide bridge.

In some embodiments of the present disclosure, the MHC class I molecule comprises or consists of at least amino acids 1-125 of the amino acid sequence according to SEQ ID NO: 1 or 2, such as at least amino acids 1-140 of the amino acid sequence according to SEQ ID NO: 1 or 2, such as at least amino acids 1-160 of the amino acid sequence according to SEQ ID NO: 1 or 2, such as at least amino acids 1-180 of the amino acid sequence according to SEQ ID NO: 1 or 2, such as at least amino acids 1-200 of the amino acid sequence according to SEQ ID NO: 1 or 2, such as at least amino acids 1-220 of the amino acid sequence according to SEQ ID NO: 1 or 2, such as at least amino acids 1-240 of the amino acid sequence according to SEQ ID NO: 1 or 2, such as at least amino acids 1-260 of the amino acid sequence according to SEQ ID NO: 1 or 2, such as at least amino acids 1-276 of the amino acid sequence according to SEQ ID NO: 1 or 2, or a variant of said MHC class I molecule having at least 85% sequence homology or identity thereto, such as at least 86% sequence homology or identity thereto, such as at least 87% sequence homology or identity thereto, such as at least 88% sequence homology or identity thereto, such as at least 89% sequence homology or identity thereto, such as at least 90% sequence homology or identity thereto, such as at least 91% sequence homology or identity thereto, such as at least 92% sequence homology or identity thereto, such as at least 93% sequence homology or identity thereto, such as at least 94% sequence homology or identity thereto, such as at least 95% sequence homology or identity thereto, such as at least 96% sequence homology or identity thereto, such as at least 97% sequence homology or identity thereto, such as at least 98% sequence homology or identity thereto, or such as at least 99% sequence homology or identity thereto,
with the proviso that the amino acids corresponding to positions
   i) 4 and 102 of SEQ ID NO: 1 or 2, and
   ii) 12 and 94 of SEQ ID NO: 1 or 2, and
   iv) 115 and 125 of SEQ ID NO: 1 or 2
have been mutated to cysteines and linked by a disulphide bridge.

In some embodiments of the present disclosure, the MHC class I molecule comprises or consists of at least amino acids 1-125 of the amino acid sequence according to SEQ ID NO: 1 or 2, such as at least amino acids 1-140 of the amino acid sequence according to SEQ ID NO: 1 or 2, such as at least amino acids 1-160 of the amino acid sequence according to SEQ ID NO: 1 or 2, such as at least amino acids 1-180 of the amino acid sequence according to SEQ ID NO: 1 or 2, such as at least amino acids 1-200 of the amino acid sequence according to SEQ ID NO: 1 or 2, such as at least amino acids 1-220 of the amino acid sequence according to SEQ ID NO: 1 or 2, such as at least amino acids 1-240 of the amino acid sequence according to SEQ ID NO: 1 or 2, such as at least amino acids 1-260 of the amino acid sequence according to SEQ ID NO: 1 or 2, such as at least amino acids 1-276 of the amino acid sequence according to SEQ ID NO: 1 or 2, or a variant of said MHC class I molecule having at least 85% sequence homology or identity thereto, such as at least 86% sequence homology or identity thereto, such as at least 87% sequence homology or identity thereto, such as at least 88% sequence homology or identity thereto, such as at least 89% sequence homology or identity thereto, such as at least 90% sequence homology or identity thereto, such as at least 91% sequence homology or identity thereto, such as at least 92% sequence homology or identity thereto, such as at least 93% sequence homology or identity thereto, such as at least 94% sequence homology or identity thereto, such as at least 95% sequence homology or identity thereto, such as at least 96% sequence homology or identity thereto, such as at least 97% sequence homology or identity thereto, such as at least 98% sequence homology or identity thereto, or such as at least 99% sequence homology or identity thereto,
with the proviso that the amino acids corresponding to positions
   i) 4 and 102 of SEQ ID NO: 1 or 2, and
   iii) 23 and 37 of SEQ ID NO: 1 or 2, and
   iv) 115 and 125 of SEQ ID NO: 1 or 2
have been mutated to cysteines and linked by a disulphide bridge.

In some embodiments of the present disclosure, the MHC class I molecule comprises or consists of at least amino acids 1-125 of the amino acid sequence according to SEQ ID NO: 1 or 2, such as at least amino acids 1-140 of the amino acid sequence according to SEQ ID NO: 1 or 2, such as at least amino acids 1-160 of the amino acid sequence according to SEQ ID NO: 1 or 2, such as at least amino acids 1-180 of the amino acid sequence according to SEQ ID NO: 1 or 2, such as at least amino acids 1-200 of the amino acid sequence according to SEQ ID NO: 1 or 2, such as at least amino acids 1-220 of the amino acid sequence according to SEQ ID NO: 1 or 2, such as at least amino acids 1-240 of the amino acid sequence according to SEQ ID NO: 1 or 2, such as at least amino acids 1-260 of the amino acid sequence according to SEQ ID NO: 1 or 2, such as at least amino acids 1-276 of the amino acid sequence according to SEQ ID NO: 1 or 2, or a variant of said MHC class I molecule having at least 85% sequence homology or identity thereto, such as at least 86% sequence homology or identity thereto, such as at least 87% sequence homology or identity thereto, such as at least 88% sequence homology or identity thereto, such as at least 89% sequence homology or identity thereto, such as at least 90% sequence homology or identity thereto, such as at least 91% sequence homology or identity thereto, such as at least 92% sequence homology or identity thereto, such as at least 93% sequence homology or identity thereto, such as at least 94% sequence homology or identity thereto, such as at least 95% sequence homology or identity thereto, such as at least 96% sequence homology or identity thereto, such as at least 97% sequence homology or identity thereto, such as at least 98% sequence homology or identity thereto, or such as at least 99% sequence homology or identity thereto,
with the proviso that the amino acids corresponding to positions
   ii) 12 and 94 of SEQ ID NO: 1 or 2,
   iii) 23 and 37 of SEQ ID NO: 1 or 2,
   iv) 115 and 125 of SEQ ID NO: 1 or 2
have been mutated to cysteines and linked by a disulphide bridge.

In some embodiments of the present disclosure, the MHC class I molecule comprises or consists of at least amino acids 1-125 of the amino acid sequence according to SEQ ID NO: 1 or 2, such as at least amino acids 1-140 of the amino acid sequence according to SEQ ID NO: 1 or 2, such as at least amino acids 1-160 of the amino acid sequence according to SEQ ID NO: 1 or 2, such as at least amino acids 1-180 of the amino acid sequence according to SEQ ID NO: 1 or 2, such as at least amino acids 1-200 of the amino acid sequence according to SEQ ID NO: 1 or 2, such as at least amino acids 1-220 of the amino acid sequence according to SEQ ID NO: 1 or 2, such as at least amino acids 1-240 of the amino acid sequence according to SEQ ID NO: 1 or 2, such as at least amino acids 1-260 of the amino acid sequence according to SEQ ID NO: 1 or 2, such as at least amino acids 1-276 of the amino acid sequence according to SEQ ID NO: 1 or 2, or a variant of said MHC class I molecule having at least 85% sequence homology or identity thereto, such as at least 86% sequence homology or identity thereto, such as at least 87% sequence homology or identity thereto, such as at least 88% sequence homology or identity thereto, such as at least 89% sequence homology or identity thereto, such as at least 90% sequence homology or identity thereto, such as at least 91% sequence homology or identity thereto, such as at least 92% sequence homology or identity thereto, such as at least 93% sequence homology or identity thereto, such as at least 94% sequence homology or identity thereto, such as at least 95% sequence homology or identity thereto, such as at least 96% sequence homology or identity thereto, such as at least 97% sequence homology or identity thereto, such as at least 98% sequence homology or identity thereto, or such as at least 99% sequence homology or identity thereto,
with the proviso that the amino acids corresponding to positions
   i) 4 and 102 of SEQ ID NO: 1 or 2, and
   ii) 12 and 94 of SEQ ID NO: 1 or 2, and
   iii) 23 and 37 of SEQ ID NO: 1 or 2, and
   iv) 115 and 125 of SEQ ID NO: 1 or 2
have been mutated to cysteines and linked by a disulphide bridge.

In some embodiments of the present disclosure, the MHC class I molecule comprises or consists of at the most amino acids 1-37 of the amino acid sequence according to SEQ ID NO: 1 or 2, such as at the most amino acids 1-45 of the amino acid sequence according to SEQ ID NO: 1 or 2, such as at the most amino acids 1-60 of the amino acid sequence according to SEQ ID NO: 1 or 2, such as at the most amino acids 1-80 of the amino acid sequence according to SEQ ID NO: 1 or 2, such as at the most amino acids 1-100 of the amino acid sequence according to SEQ ID NO: 1 or 2, such as at the most amino acids 1-120 of the amino acid sequence according to SEQ ID NO: 1 or 2, such as at the most amino acids 1-140 of the amino acid sequence according to SEQ ID NO: 1 or 2, such as at the most amino acids 1-160 of the amino acid sequence according to SEQ ID NO: 1 or 2, such as at the most amino acids 1-180 of the amino acid sequence according to SEQ ID NO: 1 or 2, such as at the most amino acids 1-200 of the amino acid sequence according to SEQ ID NO: 1 or 2, such as at the most amino acids 1-220 of the amino acid sequence according to SEQ ID NO: 1 or 2, such as at the most amino acids 1-240 of the amino acid sequence according to SEQ ID NO: 1 or 2, such as at the most amino acids 1-260 of the amino acid sequence according to SEQ ID NO: 1 or 2, such as at the most amino acids 1-276 of the amino acid sequence according to SEQ ID NO: 1 or 2, or a variant of said MHC class I molecule having at least 85% sequence homology or identity thereto, such as at least 86% sequence homology or identity thereto, such as at least 87% sequence homology or identity thereto, such as at least 88% sequence homology or identity thereto, such as at least 89% sequence homology or identity thereto, such as at least 90% sequence homology or identity thereto, such as at least 91% sequence homology or identity thereto, such as at least 92% sequence homology or identity thereto, such as at least 93% sequence homology or identity thereto, such as at least 94% sequence homology or identity thereto, such as at least 95% sequence homology or identity thereto, such as at least 96% sequence homology or identity thereto, such as at least 97% sequence homology or identity thereto, such as at least 98% sequence homology or identity thereto, or such as at least 99% sequence homology or identity thereto,
with the proviso that the amino acids corresponding to positions
   i) 4 and 102 of SEQ ID NO: 1 or 2, and/or
   ii) 12 and 94 of SEQ ID NO: 1 or 2, and/or
   iii) 23 and 37 of SEQ ID NO: 1 or 2, and/or
   iv) 115 and 125 of SEQ ID NO: 1 or 2
have been mutated to cysteines and linked by a disulphide bridge.

In some embodiments of the present disclosure, the MHC class I molecule comprises or consists of at the most amino acids 1-102 of the amino acid sequence according to SEQ ID NO: 1 or 2, such as at the most amino acids 1-120 of the amino acid sequence according to SEQ ID NO: 1 or 2, such as at the most amino acids 1-140 of the amino acid sequence according to SEQ ID NO: 1 or 2, such as at the most amino acids 1-160 of the amino acid sequence according to SEQ ID NO: 1 or 2, such as at the most amino acids 1-180 of the amino acid sequence according to SEQ ID NO: 1 or 2, such as at the most amino acids 1-200 of the amino acid sequence according to SEQ ID NO: 1 or 2, such as at the most amino acids 1-220 of the amino acid sequence according to SEQ ID NO: 1 or 2, such as at the most amino acids 1-240 of the amino acid sequence according to SEQ ID NO: 1 or 2, such as at the most amino acids 1-260 of the amino acid sequence according to SEQ ID NO: 1 or 2, such as at the most amino acids 1-276 of the amino acid sequence according to SEQ ID NO: 1 or 2, or a variant of said MHC class I molecule having at least 85% sequence homology or identity thereto, such as at least 86% sequence homology or identity thereto, such as at least 87% sequence homology or identity thereto, such as at least 88% sequence homology or identity thereto, such as at least 89% sequence homology or identity thereto, such as at least 90% sequence homology or identity thereto, such as at least 91% sequence homology or identity thereto, such as at least 92% sequence homology or identity thereto, such as at least 93% sequence homology or identity thereto, such as at least 94% sequence homology or identity thereto, such as at least 95% sequence homology or identity thereto, such as at least 96% sequence homology or identity thereto, such as at least 97% sequence homology or identity thereto, such as at least 98% sequence homology or identity thereto, or such as at least 99% sequence homology or identity thereto,
with the proviso that the amino acids corresponding to positions
   i) 4 and 102 of SEQ ID NO: 1 or 2,
have been mutated to cysteines and linked by a disulphide bridge.

In some embodiments of the present disclosure, the MHC class I molecule comprises or consists of at the most amino acids 1-94 of the amino acid sequence according to SEQ ID NO: 1 or 2, such as at the most amino acids 1-100 of the amino acid sequence according to SEQ ID NO: 1 or 2, such as at the most amino acids 1-120 of the amino acid sequence according to SEQ ID NO: 1 or 2, such as at the most amino acids 1-140 of the amino acid sequence according to SEQ ID NO: 1 or 2, such as at the most amino acids 1-160 of the amino acid sequence according to SEQ ID NO: 1 or 2, such as at the most amino acids 1-180 of the amino acid sequence according to SEQ ID NO: 1 or 2, such as at the most amino acids 1-200 of the amino acid sequence according to SEQ ID NO: 1 or 2, such as at the most amino acids 1-220 of the amino acid sequence according to SEQ ID NO: 1 or 2, such as at the most amino acids 1-240 of the amino acid sequence according to SEQ ID NO: 1 or 2, such as at the most amino acids 1-260 of the amino acid sequence according to SEQ ID NO: 1 or 2, such as at the most amino acids 1-276 of the amino acid sequence according to SEQ ID NO: 1 or 2, or a variant of said MHC class I molecule having at least 85% sequence homology or identity thereto, such as at least 86% sequence homology or identity thereto, such as at least 87% sequence homology or identity thereto, such as at least 88% sequence homology or identity thereto, such as at least 89% sequence homology or identity thereto, such as at least 90% sequence homology or identity thereto, such as at least 91% sequence homology or identity thereto, such as at least 92% sequence homology or identity thereto, such as at least 93% sequence homology or identity thereto, such as at least 94% sequence homology or identity thereto, such as at least 95% sequence homology or identity thereto, such as at least 96% sequence homology or identity thereto, such as at least 97% sequence homology or identity thereto, such as at least 98% sequence homology or identity thereto, or such as at least 99% sequence homology or identity thereto,
with the proviso that the amino acids corresponding to positions
   ii) 12 and 94 of SEQ ID NO: 1 or 2,
have been mutated to cysteines and linked by a disulphide bridge.

In some embodiments of the present disclosure, the MHC class I molecule comprises or consists of at the most amino acids 1-37 of the amino acid sequence according to SEQ ID NO: 1 or 2, such as at the most amino acids 1-45 of the amino acid sequence according to SEQ ID NO: 1 or 2, such as at the most amino acids 1-60 of the amino acid sequence according to SEQ ID NO: 1 or 2, such as at the most amino acids 1-80 of the amino acid sequence according to SEQ ID NO: 1 or 2, such as at the most amino acids 1-100 of the amino acid sequence according to SEQ ID NO: 1 or 2, such as at the most amino acids 1-120 of the amino acid sequence according to SEQ ID NO: 1 or 2, such as at the most amino acids 1-140 of the amino acid sequence according to SEQ ID NO: 1 or 2, such as at the most amino acids 1-160 of the amino acid sequence according to SEQ ID NO: 1 or 2, such as at the most amino acids 1-180 of the amino acid sequence according to SEQ ID NO: 1 or 2, such as at the most amino acids 1-200 of the amino acid sequence according to SEQ ID NO: 1 or 2, such as at the most amino acids 1-220 of the amino acid sequence according to SEQ ID NO: 1 or 2, such as at the most amino acids 1-240 of the amino acid sequence according to SEQ ID NO: 1 or 2, such as at the most amino acids 1-260 of the amino acid sequence according to SEQ ID NO: 1 or 2, such as at the most amino acids 1-276 of the amino acid sequence according to SEQ ID NO: 1 or 2, or a variant of said MHC class I molecule having at least 85% sequence homology or identity thereto, such as at least 86% sequence homology or identity thereto, such as at least 87% sequence homology or identity thereto, such as at least 88% sequence homology or identity thereto, such as at least 89% sequence homology or identity thereto, such as at least 90% sequence homology or identity thereto, such as at least 91% sequence homology or identity thereto, such as at least 92% sequence homology or identity thereto, such as at least 93% sequence homology or identity thereto, such as at least 94% sequence homology or identity thereto, such as at least 95% sequence homology or identity thereto, such as at least 96% sequence homology or identity thereto, such as at least 97% sequence homology or identity thereto, such as at least 98% sequence homology or identity thereto, or such as at least 99% sequence homology or identity thereto,
with the proviso that the amino acids corresponding to positions
   iii) 23 and 37 of SEQ ID NO: 1 or 2,
have been mutated to cysteines and linked by a disulphide bridge.

In some embodiments of the present disclosure, the MHC class I molecule comprises or consists of at the most amino acids 1-115 of the amino acid sequence according to SEQ ID NO: 1 or 2, such as at the most amino acids 1-120 of the amino acid sequence according to SEQ ID NO: 1 or 2, such as at the most amino acids 1-140 of the amino acid sequence according to SEQ ID NO: 1 or 2, such as at the most amino acids 1-160 of the amino acid sequence according to SEQ ID NO: 1 or 2, such as at the most amino acids 1-180 of the amino acid sequence according to SEQ ID NO: 1 or 2, such as at the most amino acids 1-200 of the amino acid sequence according to SEQ ID NO: 1 or 2, such as at the most amino acids 1-220 of the amino acid sequence according to SEQ ID NO: 1 or 2, such as at the most amino acids 1-240 of the amino acid sequence according to SEQ ID NO: 1 or 2, such as at the most amino acids 1-260 of the amino acid sequence according to SEQ ID NO: 1 or 2, such as at the most amino acids 1-276 of the amino acid sequence according to SEQ ID NO: 1 or 2, or a variant of said MHC class I molecule having at least 85% sequence homology or identity thereto, such as at least 86% sequence homology or identity thereto, such as at least 87% sequence homology or identity thereto, such as at least 88% sequence homology or identity thereto, such as at least 89% sequence homology or identity thereto, such as at least 90% sequence homology or identity thereto, such as at least 91% sequence homology or identity thereto, such as at least 92% sequence homology or identity thereto, such as at least 93% sequence homology or identity thereto, such as at least 94% sequence homology or identity thereto, such as at least 95% sequence homology or identity thereto, such as at least 96% sequence homology or identity thereto, such as at least 97% sequence homology or identity thereto, such as at least 98% sequence homology or identity thereto, or such as at least 99% sequence homology or identity thereto,
with the proviso that the amino acids corresponding to positions
   iv) 115 and 125 of SEQ ID NO: 1 or 2
have been mutated to cysteines and linked by a disulphide bridge.

In some embodiments of the present disclosure, the MHC class I molecule comprises or consists of at the most amino acids 1-102 of the amino acid sequence according to SEQ ID NO: 1 or 2, such as at the most amino acids 1-120 of the amino acid sequence according to SEQ ID NO: 1 or 2, such as at the most amino acids 1-140 of the amino acid sequence according to SEQ ID NO: 1 or 2, such as at the most amino acids 1-160 of the amino acid sequence according to SEQ ID NO: 1 or 2, such as at the most amino acids 1-180 of the amino acid sequence according to SEQ ID NO: 1 or 2, such as at the most amino acids 1-200 of the amino acid sequence according to SEQ ID NO: 1 or 2, such as at the most amino acids 1-220 of the amino acid sequence according to SEQ ID NO: 1 or 2, such as at the most amino acids 1-240 of the amino acid sequence according to SEQ ID NO: 1 or 2, such as at the most amino acids 1-260 of the amino acid sequence according to SEQ ID NO: 1 or 2, such as at the most amino acids 1-276 of the amino acid sequence according to SEQ ID NO: 1 or 2, or a variant of said MHC class I molecule having at least 85% sequence homology or identity thereto, such as at least 86% sequence homology or identity thereto, such as at least 87% sequence homology or identity thereto, such as at least 88% sequence homology or identity thereto, such as at least 89% sequence homology or identity thereto, such as at least 90% sequence homology or identity thereto, such as at least 91% sequence homology or identity thereto, such as at least 92% sequence homology or identity thereto, such as at least 93% sequence homology or identity thereto, such as at least 94% sequence homology or identity thereto, such as at least 95% sequence homology or identity thereto, such as at least 96% sequence homology or identity thereto, such as at least 97% sequence homology or identity thereto, such as at least 98% sequence homology or identity thereto, or such as at least 99% sequence homology or identity thereto,
with the proviso that the amino acids corresponding to positions
   i) 4 and 102 of SEQ ID NO: 1 or 2, and
   ii) 12 and 94 of SEQ ID NO: 1 or 2,
have been mutated to cysteines and linked by a disulphide bridge.

In some embodiments of the present disclosure, the MHC class I molecule comprises or consists of at the most amino acids 1-102 of the amino acid sequence according to SEQ ID NO: 1 or 2, such as at the most amino acids 1-120 of the amino acid sequence according to SEQ ID NO: 1 or 2, such as at the most amino acids 1-140 of the amino acid sequence according to SEQ ID NO: 1 or 2, such as at the most amino acids 1-160 of the amino acid sequence according to SEQ ID NO: 1 or 2, such as at the most amino acids 1-180 of the amino acid sequence according to SEQ ID NO: 1 or 2, such as at the most amino acids 1-200 of the amino acid sequence according to SEQ ID NO: 1 or 2, such as at the most amino acids 1-220 of the amino acid sequence according to SEQ ID NO: 1 or 2, such as at the most amino acids 1-240 of the amino acid sequence according to SEQ ID NO: 1 or 2, such as at the most amino acids 1-260 of the amino acid sequence according to SEQ ID NO: 1 or 2, such as at the most amino acids 1-276 of the amino acid sequence according to SEQ ID NO: 1 or 2, or a variant of said MHC class I molecule having at least 85% sequence homology or identity thereto, such as at least 86% sequence homology or identity thereto, such as at least 87% sequence homology or identity thereto, such as at least 88% sequence homology or identity thereto, such as at least 89% sequence homology or identity thereto, such as at least 90% sequence homology or identity thereto, such as at least 91% sequence homology or identity thereto, such as at least 92% sequence homology or identity thereto, such as at least 93% sequence homology or identity thereto, such as at least 94% sequence homology or identity thereto, such as at least 95% sequence homology or identity thereto, such as at least 96% sequence homology or identity thereto, such as at least 97% sequence homology or identity thereto, such as at least 98% sequence homology or identity thereto, or such as at least 99% sequence homology or identity thereto,
with the proviso that the amino acids corresponding to positions
   i) 4 and 102 of SEQ ID NO: 1 or 2, and
   iii) 23 and 37 of SEQ ID NO: 1 or 2,
have been mutated to cysteines and linked by a disulphide bridge.

In some embodiments of the present disclosure, the MHC class I molecule comprises or consists of at the most amino acids 1-125 of the amino acid sequence according to SEQ ID NO: 1 or 2, such as at the most amino acids 1-140 of the amino acid sequence according to SEQ ID NO: 1 or 2, such as at the most amino acids 1-160 of the amino acid sequence according to SEQ ID NO: 1 or 2, such as at the most amino acids 1-180 of the amino acid sequence according to SEQ ID NO: 1 or 2, such as at the most amino acids 1-200 of the amino acid sequence according to SEQ ID NO: 1 or 2, such as at the most amino acids 1-220 of the amino acid sequence according to SEQ ID NO: 1 or 2, such as at the most amino acids 1-240 of the amino acid sequence according to SEQ ID NO: 1 or 2, such as at the most amino acids 1-260 of the amino acid sequence according to SEQ ID NO: 1 or 2, such as at the most amino acids 1-276 of the amino acid sequence according to SEQ ID NO: 1 or 2, or a variant of said MHC class I molecule having at least 85% sequence homology or identity thereto, such as at least 86% sequence homology or identity thereto, such as at least 87% sequence homology or identity thereto, such as at least 88% sequence homology or identity thereto, such as at least 89% sequence homology or identity thereto, such as at least 90% sequence homology or identity thereto, such as at least 91% sequence homology or identity thereto, such as at least 92% sequence homology or identity thereto, such as at least 93% sequence homology or identity thereto, such as at least 94% sequence homology or identity thereto, such as at least 95% sequence homology or identity thereto, such as at least 96% sequence homology or identity thereto, such as at least 97% sequence homology or identity thereto, such as at least 98% sequence homology or identity thereto, or such as at least 99% sequence homology or identity thereto,
with the proviso that the amino acids corresponding to positions
   i) 4 and 102 of SEQ ID NO: 1 or 2, and
   iv) 115 and 125 of SEQ ID NO: 1 or 2
have been mutated to cysteines and linked by a disulphide bridge.

In some embodiments of the present disclosure, the MHC class I molecule comprises or consists of at the most amino acids 1-94 of the amino acid sequence according to SEQ ID NO: 1 or 2, such as at the most amino acids 1-100 of the amino acid sequence according to SEQ ID NO: 1 or 2, such as at the most amino acids 1-120 of the amino acid sequence according to SEQ ID NO: 1 or 2, such as at the most amino acids 1-140 of the amino acid sequence according to SEQ ID NO: 1 or 2, such as at the most amino acids 1-160 of the amino acid sequence according to SEQ ID NO: 1 or 2, such as at the most amino acids 1-180 of the amino acid sequence according to SEQ ID NO: 1 or 2, such as at the most amino acids 1-200 of the amino acid sequence according to SEQ ID NO: 1 or 2, such as at the most amino acids 1-220 of the amino acid sequence according to SEQ ID NO: 1 or 2, such as at the most amino acids 1-240 of the amino acid sequence according to SEQ ID NO: 1 or 2, such as at the most amino acids 1-260 of the amino acid sequence according to SEQ ID NO: 1 or 2, such as at the most amino acids 1-276 of the amino acid sequence according to SEQ ID NO: 1 or 2, or a variant of said MHC class I molecule having at least 85% sequence homology or identity thereto, such as at least 86% sequence homology or identity thereto, such as at least 87% sequence homology or identity thereto, such as at least 88% sequence homology or identity thereto, such as at least 89% sequence homology or identity thereto, such as at least 90% sequence homology or identity thereto, such as at least 91% sequence homology or identity thereto, such as at least 92% sequence homology or identity thereto, such as at least 93% sequence homology or identity thereto, such as at least 94% sequence homology or identity thereto, such as at least 95% sequence homology or identity thereto, such as at least 96% sequence homology or identity thereto, such as at least 97% sequence homology or identity thereto, such as at least 98% sequence homology or identity thereto, or such as at least 99% sequence homology or identity thereto,
with the proviso that the amino acids corresponding to positions
   ii) 12 and 94 of SEQ ID NO: 1 or 2, and
   iii) 23 and 37 of SEQ ID NO: 1 or 2,
have been mutated to cysteines and linked by a disulphide bridge.

In some embodiments of the present disclosure, the MHC class I molecule comprises or consists of at the most amino acids 1-125 of the amino acid sequence according to SEQ ID NO: 1 or 2, such as at the most amino acids 1-140 of the amino acid sequence according to SEQ ID NO: 1 or 2, such as at the most amino acids 1-160 of the amino acid sequence according to SEQ ID NO: 1 or 2, such as at the most amino acids 1-180 of the amino acid sequence according to SEQ ID NO: 1 or 2, such as at the most amino acids 1-200 of the amino acid sequence according to SEQ ID NO: 1 or 2, such as at the most amino acids 1-220 of the amino acid sequence according to SEQ ID NO: 1 or 2, such as at the most amino acids 1-240 of the amino acid sequence according to SEQ ID NO: 1 or 2, such as at the most amino acids 1-260 of the amino acid sequence according to SEQ ID NO: 1 or 2, such as at the most amino acids 1-276 of the amino acid sequence according to SEQ ID NO: 1 or 2, or a variant of said MHC class I molecule having at least 85% sequence homology or identity thereto, such as at least 86% sequence homology or identity thereto, such as at least 87% sequence homology or identity thereto, such as at least 88% sequence homology or identity thereto, such as at least 89% sequence homology or identity thereto, such as at least 90% sequence homology or identity thereto, such as at least 91% sequence homology or identity thereto, such as at least 92% sequence homology or identity thereto, such as at least 93% sequence homology or identity thereto, such as at least 94% sequence homology or identity thereto, such as at least 95% sequence homology or identity thereto, such as at least 96% sequence homology or identity thereto, such as at least 97% sequence homology or identity thereto, such as at least 98% sequence homology or identity thereto, or such as at least 99% sequence homology or identity thereto,
with the proviso that the amino acids corresponding to positions
   ii) 12 and 94 of SEQ ID NO: 1 or 2,
   iv) 115 and 125 of SEQ ID NO: 1 or 2
have been mutated to cysteines and linked by a disulphide bridge.

In some embodiments of the present disclosure, the MHC class I molecule comprises or consists of at the most amino acids 1-125 of the amino acid sequence according to SEQ ID NO: 1 or 2, such as at the most amino acids 1-140 of the amino acid sequence according to SEQ ID NO: 1 or 2, such as at the most amino acids 1-160 of the amino acid sequence according to SEQ ID NO: 1 or 2, such as at the most amino acids 1-180 of the amino acid sequence according to SEQ ID NO: 1 or 2, such as at the most amino acids 1-200 of the amino acid sequence according to SEQ ID NO: 1 or 2, such as at the most amino acids 1-220 of the amino acid sequence according to SEQ ID NO: 1 or 2, such as at the most amino acids 1-240 of the amino acid sequence according to SEQ ID NO: 1 or 2, such as at the most amino acids 1-260 of the amino acid sequence according to SEQ ID NO: 1 or 2, such as at the most amino acids 1-276 of the amino acid sequence according to SEQ ID NO: 1 or 2, or a variant of said MHC class I molecule having at least 85% sequence homology or identity thereto, such as at least 86% sequence homology or identity thereto, such as at least 87% sequence homology or identity thereto, such as at least 88% sequence homology or identity thereto, such as at least 89% sequence homology or identity thereto, such as at least 90% sequence homology or identity thereto, such as at least 91% sequence homology or identity thereto, such as at least 92% sequence homology or identity thereto, such as at least 93% sequence homology or identity thereto, such as at least 94% sequence homology or identity thereto, such as at least 95% sequence homology or identity thereto, such as at least 96% sequence homology or identity thereto, such as at least 97% sequence homology or identity thereto, such as at least 98% sequence homology or identity thereto, or such as at least 99% sequence homology or identity thereto,
with the proviso that the amino acids corresponding to positions
   iii) 23 and 37 of SEQ ID NO: 1 or 2,
   iv) 115 and 125 of SEQ ID NO: 1 or 2
have been mutated to cysteines and linked by a disulphide bridge.

In some embodiments of the present disclosure, the MHC class I molecule comprises or consists of at the most amino acids 1-102 of the amino acid sequence according to SEQ ID NO: 1 or 2, such as at the most amino acids 1-120 of the amino acid sequence according to SEQ ID NO: 1 or 2, such as at the most amino acids 1-140 of the amino acid sequence according to SEQ ID NO: 1 or 2, such as at the most amino acids 1-160 of the amino acid sequence according to SEQ ID NO: 1 or 2, such as at the most amino acids 1-180 of the amino acid sequence according to SEQ ID NO: 1 or 2, such as at the most amino acids 1-200 of the amino acid sequence according to SEQ ID NO: 1 or 2, such as at the most amino acids 1-220 of the amino acid sequence according to SEQ ID NO: 1 or 2, such as at the most amino acids 1-240 of the amino acid sequence according to SEQ ID NO: 1 or 2, such as at the most amino acids 1-260 of the amino acid sequence according to SEQ ID NO: 1 or 2, such as at the most amino acids 1-276 of the amino acid sequence according to SEQ ID NO: 1 or 2, or a variant of said MHC class I molecule having at least 85% sequence homology or identity thereto, such as at least 86% sequence homology or identity thereto, such as at least 87% sequence homology or identity thereto, such as at least 88% sequence homology or identity thereto, such as at least 89% sequence homology or identity thereto, such as at least 90% sequence homology or identity thereto, such as at least 91% sequence homology or identity thereto, such as at least 92% sequence homology or identity thereto, such as at least 93% sequence homology or identity thereto, such as at least 94% sequence homology or identity thereto, such as at least 95% sequence homology or identity thereto, such as at least 96% sequence homology or identity thereto, such as at least 97% sequence homology or identity thereto, such as at least 98% sequence homology or identity thereto, or such as at least 99% sequence homology or identity thereto,
with the proviso that the amino acids corresponding to positions
   i) 4 and 102 of SEQ ID NO: 1 or 2, and
   ii) 12 and 94 of SEQ ID NO: 1 or 2, and
   iii) 23 and 37 of SEQ ID NO: 1 or 2,
have been mutated to cysteines and linked by a disulphide bridge.

In some embodiments of the present disclosure, the MHC class I molecule comprises or consists of at the most amino acids 1-125 of the amino acid sequence according to SEQ ID NO: 1 or 2, such as at the most amino acids 1-140 of the amino acid sequence according to SEQ ID NO: 1 or 2, such as at the most amino acids 1-160 of the amino acid sequence according to SEQ ID NO: 1 or 2, such as at the most amino acids 1-180 of the amino acid sequence according to SEQ ID NO: 1 or 2, such as at the most amino acids 1-200 of the amino acid sequence according to SEQ ID NO: 1 or 2, such as at the most amino acids 1-220 of the amino acid sequence according to SEQ ID NO: 1 or 2, such as at the most amino acids 1-240 of the amino acid sequence according to SEQ ID NO: 1 or 2, such as at the most amino acids 1-260 of the amino acid sequence according to SEQ ID NO: 1 or 2, such as at the most amino acids 1-276 of the amino acid sequence according to SEQ ID NO: 1 or 2, or a variant of said MHC class I molecule having at least 85% sequence homology or identity thereto, such as at least 86% sequence homology or identity thereto, such as at least 87% sequence homology or identity thereto, such as at least 88% sequence homology or identity thereto, such as at least 89% sequence homology or identity thereto, such as at least 90% sequence homology or identity thereto, such as at least 91% sequence homology or identity thereto, such as at least 92% sequence homology or identity thereto, such as at least 93% sequence homology or identity thereto, such as at least 94% sequence homology or identity thereto, such as at least 95% sequence homology or identity thereto, such as at least 96% sequence homology or identity thereto, such as at least 97% sequence homology or identity thereto, such as at least 98% sequence homology or identity thereto, or such as at least 99% sequence homology or identity thereto,
with the proviso that the amino acids corresponding to positions
   i) 4 and 102 of SEQ ID NO: 1 or 2, and
   ii) 12 and 94 of SEQ ID NO: 1 or 2, and
   iv) 115 and 125 of SEQ ID NO: 1 or 2 have been mutated to cysteines and linked by a disulphide bridge.

In some embodiments of the present disclosure, the MHC class I molecule comprises or consists of at the most amino acids 1-125 of the amino acid sequence according to SEQ ID NO: 1 or 2, such as at the most amino acids 1-140 of the amino acid sequence according to SEQ ID NO: 1 or 2, such as at the most amino acids 1-160 of the amino acid sequence according to SEQ ID NO: 1 or 2, such as at the most amino acids 1-180 of the amino acid sequence according to SEQ ID NO: 1 or 2, such as at the most amino acids 1-200 of the amino acid sequence according to SEQ ID NO: 1 or 2, such as at the most amino acids 1-220 of the amino acid sequence according to SEQ ID NO: 1 or 2, such as at the most amino acids 1-240 of the amino acid sequence according to SEQ ID NO: 1 or 2, such as at the most amino acids 1-260 of the amino acid sequence according to SEQ ID NO: 1 or 2, such as at the most amino acids 1-276 of the amino acid sequence according to SEQ ID NO: 1 or 2, or a variant of said MHC class I molecule having at least 85% sequence homology or identity thereto, such as at least 86% sequence homology or identity thereto, such as at least 87% sequence homology or identity thereto, such as at least 88% sequence homology or identity thereto, such as at least 89% sequence homology or identity thereto, such as at least 90% sequence homology or identity thereto, such as at least 91% sequence homology or identity thereto, such as at least 92% sequence homology or identity thereto, such as at least 93% sequence homology or identity thereto, such as at least 94% sequence homology or identity thereto, such as at least 95% sequence homology or identity thereto, such as at least 96% sequence homology or identity thereto, such as at least 97% sequence homology or identity thereto, such as at least 98% sequence homology or identity thereto, or such as at least 99% sequence homology or identity thereto,
with the proviso that the amino acids corresponding to positions
   i) 4 and 102 of SEQ ID NO: 1 or 2, and
   iii) 23 and 37 of SEQ ID NO: 1 or 2, and
   iv) 115 and 125 of SEQ ID NO: 1 or 2
have been mutated to cysteines and linked by a disulphide bridge.

In some embodiments of the present disclosure, the MHC class I molecule comprises or consists of at the most amino acids 1-125 of the amino acid sequence according to SEQ ID NO: 1 or 2, such as at the most amino acids 1-140 of the amino acid sequence according to SEQ ID NO: 1 or 2, such as at the most amino acids 1-160 of the amino acid sequence according to SEQ ID NO: 1 or 2, such as at the most amino acids 1-180 of the amino acid sequence according to SEQ ID NO: 1 or 2, such as at the most amino acids 1-200 of the amino acid sequence according to SEQ ID NO: 1 or 2, such as at the most amino acids 1-220 of the amino acid sequence according to SEQ ID NO: 1 or 2, such as at the most amino acids 1-240 of the amino acid sequence according to SEQ ID NO: 1 or 2, such as at the most amino acids 1-260 of the amino acid sequence according to SEQ ID NO: 1 or 2, such as at the most amino acids 1-276 of the amino acid sequence according to SEQ ID NO: 1 or 2, or a variant of said MHC class I molecule having at least 85% sequence homology or identity thereto, such as at least 86% sequence homology or identity thereto, such as at least 87% sequence homology or identity thereto, such as at least 88% sequence homology or identity thereto, such as at least 89% sequence homology or identity thereto, such as at least 90% sequence homology or identity thereto, such as at least 91% sequence homology or identity thereto, such as at least 92% sequence homology or identity thereto, such as at least 93% sequence homology or identity thereto, such as at least 94% sequence homology or identity thereto, such as at least 95% sequence homology or identity thereto, such as at least 96% sequence homology or identity thereto, such as at least 97% sequence homology or identity thereto, such as at least 98% sequence homology or identity thereto, or such as at least 99% sequence homology or identity thereto,
with the proviso that the amino acids corresponding to positions
   ii) 12 and 94 of SEQ ID NO: 1 or 2,
   iii) 23 and 37 of SEQ ID NO: 1 or 2,
   iv) 115 and 125 of SEQ ID NO: 1 or 2
have been mutated to cysteines and linked by a disulphide bridge.

In some embodiments of the present disclosure, the MHC class I molecule comprises or consists of at the most amino acids 1-125 of the amino acid sequence according to SEQ ID NO: 1 or 2, such as at the most amino acids 1-140 of the amino acid sequence according to SEQ ID NO: 1 or 2, such as at the most amino acids 1-160 of the amino acid sequence according to SEQ ID NO: 1 or 2, such as at the most amino acids 1-180 of the amino acid sequence according to SEQ ID NO: 1 or 2, such as at the most amino acids 1-200 of the amino acid sequence according to SEQ ID NO: 1 or 2, such as at the most amino acids 1-220 of the amino acid sequence according to SEQ ID NO: 1 or 2, such as at the most amino acids 1-240 of the amino acid sequence according to SEQ ID NO: 1 or 2, such as at the most amino acids 1-260 of the amino acid sequence according to SEQ ID NO: 1 or 2, such as at the most amino acids 1-276 of the amino acid sequence according to SEQ ID NO: 1 or 2, or a variant of said MHC class I molecule having at least 85% sequence homology or identity thereto, such as at least 86% sequence homology or identity thereto, such as at least 87% sequence homology or identity thereto, such as at least 88% sequence homology or identity thereto, such as at least 89% sequence homology or identity thereto, such as at least 90% sequence homology or identity thereto, such as at least 91% sequence homology or identity thereto, such as at least 92% sequence homology or identity thereto, such as at least 93% sequence homology or identity thereto, such as at least 94% sequence homology or identity thereto, such as at least 95% sequence homology or identity thereto, such as at least 96% sequence homology or identity thereto, such as at least 97% sequence homology or identity thereto, such as at least 98% sequence homology or identity thereto, or such as at least 99% sequence homology or identity thereto,
with the proviso that the amino acids corresponding to positions
   i) 4 and 102 of SEQ ID NO: 1 or 2, and
   ii) 12 and 94 of SEQ ID NO: 1 or 2, and
   iii) 23 and 37 of SEQ ID NO: 1 or 2, and
   iv) 115 and 125 of SEQ ID NO: 1 or 2
have been mutated to cysteines and linked by a disulphide bridge.

In some embodiments of the present disclosure, the MHC class I molecule comprises or consists of at least amino acids 1-276 of the amino acid sequence according to SEQ ID NO: 1 or 2, or a variant of said MHC class I molecule having at least 85% sequence homology or identity thereto, such as at least 86% sequence homology or identity thereto, such as at least 87% sequence homology or identity thereto, such as at least 88% sequence homology or identity thereto, such as at least 89% sequence homology or identity thereto, such as at least 90% sequence homology or identity thereto, such as at least 91% sequence homology or identity thereto, such as at least 92% sequence homology or identity thereto, such as at least 93% sequence homology or identity thereto, such as at least 94% sequence homology or identity thereto, such as at least 95% sequence homology or identity thereto, such as at least 96% sequence homology or identity thereto, such as at least 97% sequence homology or identity thereto, such as at least 98% sequence homology or identity thereto, or such as at least 99% sequence homology or identity thereto,
with the proviso that the amino acids corresponding to positions
   i) 4 and 102 of SEQ ID NO: 1 or 2, and/or
   ii) 12 and 94 of SEQ ID NO: 1 or 2, and/or
   iii) 23 and 37 of SEQ ID NO: 1 or 2, and/or
   iv) 115 and 125 of SEQ ID NO: 1 or 2
have been mutated to cysteines and linked by a disulphide bridge.

In some embodiments of the present disclosure, the MHC class I molecule comprises or consists of at least amino acids 1-276 of the amino acid sequence according to SEQ ID NO: 1 or 2, or a variant of said MHC class I molecule having at least 85% sequence homology or identity thereto, such as at least 86% sequence homology or identity thereto, such as at least 87% sequence homology or identity thereto, such as at least 88% sequence homology or identity thereto, such as at least 89% sequence homology or identity thereto, such as at least 90% sequence homology or identity thereto, such as at least 91% sequence homology or identity thereto, such as at least 92% sequence homology or identity thereto, such as at least 93% sequence homology or identity thereto, such as at least 94% sequence homology or identity thereto, such as at least 95% sequence homology or identity thereto, such as at least 96% sequence homology or identity thereto, such as at least 97% sequence homology or identity thereto, such as at least 98% sequence homology or identity thereto, or such as at least 99% sequence homology or identity thereto,
with the proviso that the amino acids corresponding to positions
   i) 4 and 102 of SEQ ID NO: 1 or 2,
have been mutated to cysteines and linked by a disulphide bridge.

In some embodiments of the present disclosure, the MHC class I molecule comprises or consists of at least amino acids 1-276 of the amino acid sequence according to SEQ ID NO: 1 or 2, or a variant of said MHC class I molecule having at least 85% sequence homology or identity thereto, such as at least 86% sequence homology or identity thereto, such as at least 87% sequence homology or identity thereto, such as at least 88% sequence homology or identity thereto, such as at least 89% sequence homology or identity thereto, such as at least 90% sequence homology or identity thereto, such as at least 91% sequence homology or identity thereto, such as at least 92% sequence homology or identity thereto, such as at least 93% sequence homology or identity thereto, such as at least 94% sequence homology or identity thereto, such as at least 95% sequence homology or identity thereto, such as at least 96% sequence homology or identity thereto, such as at least 97% sequence homology or identity thereto, such as at least 98% sequence homology or identity thereto, or such as at least 99% sequence homology or identity thereto,
with the proviso that the amino acids corresponding to positions
   ii) 12 and 94 of SEQ ID NO: 1 or 2,
have been mutated to cysteines and linked by a disulphide bridge.

In some embodiments of the present disclosure, the MHC class I molecule comprises or consists of at least amino acids 1-276 of the amino acid sequence according to SEQ ID NO: 1 or 2, or a variant of said MHC class I molecule having at least 85% sequence homology or identity thereto, such as at least 86% sequence homology or identity thereto, such as at least 87% sequence homology or identity thereto, such as at least 88% sequence homology or identity thereto, such as at least 89% sequence homology or identity thereto, such as at least 90% sequence homology or identity thereto, such as at least 91% sequence homology or identity thereto, such as at least 92% sequence homology or identity thereto, such as at least 93% sequence homology or identity thereto, such as at least 94% sequence homology or identity thereto, such as at least 95% sequence homology or identity thereto, such as at least 96% sequence homology or identity thereto, such as at least 97% sequence homology or identity thereto, such as at least 98% sequence homology or identity thereto, or such as at least 99% sequence homology or identity thereto,
with the proviso that the amino acids corresponding to positions
   iii) 23 and 37 of SEQ ID NO: 1 or 2,
have been mutated to cysteines and linked by a disulphide bridge.

In some embodiments of the present disclosure, the MHC class I molecule comprises or consists of at least amino acids 1-276 of the amino acid sequence according to SEQ ID NO: 1 or 2, or a variant of said MHC class I molecule having at least 85% sequence homology or identity thereto, such as at least 86% sequence homology or identity thereto, such as at least 87% sequence homology or identity thereto, such as at least 88% sequence homology or identity thereto, such as at least 89% sequence homology or identity thereto, such as at least 90% sequence homology or identity thereto, such as at least 91% sequence homology or identity thereto, such as at least 92% sequence homology or identity thereto, such as at least 93% sequence homology or identity thereto, such as at least 94% sequence homology or identity thereto, such as at least 95% sequence homology or identity thereto, such as at least 96% sequence homology or identity thereto, such as at least 97% sequence homology or identity thereto, such as at least 98% sequence homology or identity thereto, or such as at least 99% sequence homology or identity thereto,
with the proviso that the amino acids corresponding to positions
   iv) 115 and 125 of SEQ ID NO: 1 or 2
have been mutated to cysteines and linked by a disulphide bridge.

In some embodiments of the present disclosure, the MHC class I molecule comprises or consists of at least amino acids 1-276 of the amino acid sequence according to SEQ ID NO: 1 or 2, or a variant of said MHC class I molecule having at least 85% sequence homology or identity thereto, such as at least 86% sequence homology or identity thereto, such as at least 87% sequence homology or identity thereto, such as at least 88% sequence homology or identity thereto, such as at least 89% sequence homology or identity thereto, such as at least 90% sequence homology or identity thereto, such as at least 91% sequence homology or identity thereto, such as at least 92% sequence homology or identity thereto, such as at least 93% sequence homology or identity thereto, such as at least 94% sequence homology or identity thereto, such as at least 95% sequence homology or identity thereto, such as at least 96% sequence homology or identity thereto, such as at least 97% sequence homology or identity thereto, such as at least 98% sequence homology or identity thereto, or such as at least 99% sequence homology or identity thereto,
with the proviso that the amino acids corresponding to positions
   i) 4 and 102 of SEQ ID NO: 1 or 2, and
   ii) 12 and 94 of SEQ ID NO: 1 or 2,
have been mutated to cysteines and linked by a disulphide bridge.

In some embodiments of the present disclosure, the MHC class I molecule comprises or consists of at least amino acids 1-276 of the amino acid sequence according to SEQ ID NO: 1 or 2, or a variant of said MHC class I molecule having at least 85% sequence homology or identity thereto, such as at least 86% sequence homology or identity thereto, such as at least 87% sequence homology or identity thereto, such as at least 88% sequence homology or identity thereto, such as at least 89% sequence homology or identity thereto, such as at least 90% sequence homology or identity thereto, such as at least 91% sequence homology or identity thereto, such as at least 92% sequence homology or identity thereto, such as at least 93% sequence homology or identity thereto, such as at least 94% sequence homology or identity thereto, such as at least 95% sequence homology or identity thereto, such as at least 96% sequence homology or identity thereto, such as at least 97% sequence homology or identity thereto, such as at least 98% sequence homology or identity thereto, or such as at least 99% sequence homology or identity thereto,
with the proviso that the amino acids corresponding to positions
   i) 4 and 102 of SEQ ID NO: 1 or 2, and
   iii) 23 and 37 of SEQ ID NO: 1 or 2,
have been mutated to cysteines and linked by a disulphide bridge.

In some embodiments of the present disclosure, the MHC class I molecule comprises or consists of at least amino acids 1-276 of the amino acid sequence according to SEQ ID NO: 1 or 2, or a variant of said MHC class I molecule having at least 85% sequence homology or identity thereto, such as at least 86% sequence homology or identity thereto, such as at least 87% sequence homology or identity thereto, such as at least 88% sequence homology or identity thereto, such as at least 89% sequence homology or identity thereto, such as at least 90% sequence homology or identity thereto, such as at least 91% sequence homology or identity thereto, such as at least 92% sequence homology or identity thereto, such as at least 93% sequence homology or identity thereto, such as at least 94% sequence homology or identity thereto, such as at least 95% sequence homology or identity thereto, such as at least 96% sequence homology or identity thereto, such as at least 97% sequence homology or identity thereto, such as at least 98% sequence homology or identity thereto, or such as at least 99% sequence homology or identity thereto,
with the proviso that the amino acids corresponding to positions
   i) 4 and 102 of SEQ ID NO: 1 or 2, and
   iv) 115 and 125 of SEQ ID NO: 1 or 2
have been mutated to cysteines and linked by a disulphide bridge.

In some embodiments of the present disclosure, the MHC class I molecule comprises or consists of at least amino acids 1-276 of the amino acid sequence according to SEQ ID NO: 1 or 2, or a variant of said MHC class I molecule having at least 85% sequence homology or identity thereto, such as at least 86% sequence homology or identity thereto, such as at least 87% sequence homology or identity thereto, such as at least 88% sequence homology or identity thereto, such as at least 89% sequence homology or identity thereto, such as at least 90% sequence homology or identity thereto, such as at least 91% sequence homology or identity thereto, such as at least 92% sequence homology or identity thereto, such as at least 93% sequence homology or identity thereto, such as at least 94% sequence homology or identity thereto, such as at least 95% sequence homology or identity thereto, such as at least 96% sequence homology or identity thereto, such as at least 97% sequence homology or identity thereto, such as at least 98% sequence homology or identity thereto, or such as at least 99% sequence homology or identity thereto,
with the proviso that the amino acids corresponding to positions
   ii) 12 and 94 of SEQ ID NO: 1 or 2, and
   iii) 23 and 37 of SEQ ID NO: 1 or 2,
have been mutated to cysteines and linked by a disulphide bridge.

In some embodiments of the present disclosure, the MHC class I molecule comprises or consists of at least amino acids 1-276 of the amino acid sequence according to SEQ ID NO: 1 or 2, or a variant of said MHC class I molecule having at least 85% sequence homology or identity thereto, such as at least 86% sequence homology or identity thereto, such as at least 87% sequence homology or identity thereto, such as at least 88% sequence homology or identity thereto, such as at least 89% sequence homology or identity thereto, such as at least 90% sequence homology or identity thereto, such as at least 91% sequence homology or identity thereto, such as at least 92% sequence homology or identity thereto, such as at least 93% sequence homology or identity thereto, such as at least 94% sequence homology or identity thereto, such as at least 95% sequence homology or identity thereto, such as at least 96% sequence homology or identity thereto, such as at least 97% sequence homology or identity thereto, such as at least 98% sequence homology or identity thereto, or such as at least 99% sequence homology or identity thereto,
with the proviso that the amino acids corresponding to positions
   ii) 12 and 94 of SEQ ID NO: 1 or 2,
   iv) 115 and 125 of SEQ ID NO: 1 or 2
have been mutated to cysteines and linked by a disulphide bridge.

In some embodiments of the present disclosure, the MHC class I molecule comprises or consists of at least amino acids 1-276 of the amino acid sequence according to SEQ ID NO: 1 or 2, or a variant of said MHC class I molecule having at least 85% sequence homology or identity thereto, such as at least 86% sequence homology or identity thereto, such as at least 87% sequence homology or identity thereto, such as at least 88% sequence homology or identity thereto, such as at least 89% sequence homology or identity thereto, such as at least 90% sequence homology or identity thereto, such as at least 91% sequence homology or identity thereto, such as at least 92% sequence homology or identity thereto, such as at least 93% sequence homology or identity thereto, such as at least 94% sequence homology or identity thereto, such as at least 95% sequence homology or identity thereto, such as at least 96% sequence homology or identity thereto, such as at least 97% sequence homology or identity thereto, such as at least 98% sequence homology or identity thereto, or such as at least 99% sequence homology or identity thereto,
with the proviso that the amino acids corresponding to positions
   iii) 23 and 37 of SEQ ID NO: 1 or 2,
   iv) 115 and 125 of SEQ ID NO: 1 or 2
have been mutated to cysteines and linked by a disulphide bridge.

In some embodiments of the present disclosure, the MHC class I molecule comprises or consists of at least amino acids 1-276 of the amino acid sequence according to SEQ ID NO: 1 or 2, or a variant of said MHC class I molecule having at least 85% sequence homology or identity thereto, such as at least 86% sequence homology or identity thereto, such as at least 87% sequence homology or identity thereto, such as at least 88% sequence homology or identity thereto, such as at least 89% sequence homology or identity thereto, such as at least 90% sequence homology or identity thereto, such as at least 91% sequence homology or identity thereto, such as at least 92% sequence homology or identity thereto, such as at least 93% sequence homology or identity thereto, such as at least 94% sequence homology or identity thereto, such as at least 95% sequence homology or identity thereto, such as at least 96% sequence homology or identity thereto, such as at least 97% sequence homology or identity thereto, such as at least 98% sequence homology or identity thereto, or such as at least 99% sequence homology or identity thereto,
with the proviso that the amino acids corresponding to positions
   i) 4 and 102 of SEQ ID NO: 1 or 2, and
   ii) 12 and 94 of SEQ ID NO: 1 or 2, and
   iii) 23 and 37 of SEQ ID NO: 1 or 2,
have been mutated to cysteines and linked by a disulphide bridge.

In some embodiments of the present disclosure, the MHC class I molecule comprises or consists of at least amino acids 1-276 of the amino acid sequence according to SEQ ID NO: 1 or 2, or a variant of said MHC class I molecule having at least 85% sequence homology or identity thereto, such as at least 86% sequence homology or identity thereto, such as at least 87% sequence homology or identity thereto, such as at least 88% sequence homology or identity thereto, such as at least 89% sequence homology or identity thereto, such as at least 90% sequence homology or identity thereto, such as at least 91% sequence homology or identity thereto, such as at least 92% sequence homology or identity thereto, such as at least 93% sequence homology or identity thereto, such as at least 94% sequence homology or identity thereto, such as at least 95% sequence homology or identity thereto, such as at least 96% sequence homology or identity thereto, such as at least 97% sequence homology or identity thereto, such as at least 98% sequence homology or identity thereto, or such as at least 99% sequence homology or identity thereto,
with the proviso that the amino acids corresponding to positions
   i) 4 and 102 of SEQ ID NO: 1 or 2, and
   ii) 12 and 94 of SEQ ID NO: 1 or 2, and
   iv) 115 and 125 of SEQ ID NO: 1 or 2
have been mutated to cysteines and linked by a disulphide bridge.

In some embodiments of the present disclosure, the MHC class I molecule comprises or consists of at least amino acids 1-276 of the amino acid sequence according to SEQ ID NO: 1 or 2, or a variant of said MHC class I molecule having at least 85% sequence homology or identity thereto, such as at least 86% sequence homology or identity thereto, such as at least 87% sequence homology or identity thereto, such as at least 88% sequence homology or identity thereto, such as at least 89% sequence homology or identity thereto, such as at least 90% sequence homology or identity thereto, such as at least 91% sequence homology or identity thereto, such as at least 92% sequence homology or identity thereto, such as at least 93% sequence homology or identity thereto, such as at least 94% sequence homology or identity thereto, such as at least 95% sequence homology or identity thereto, such as at least 96% sequence homology or identity thereto, such as at least 97% sequence homology or identity thereto, such as at least 98% sequence homology or identity thereto, or such as at least 99% sequence homology or identity thereto,
with the proviso that the amino acids corresponding to positions
   i) 4 and 102 of SEQ ID NO: 1 or 2, and
   iii) 23 and 37 of SEQ ID NO: 1 or 2, and
   iv) 115 and 125 of SEQ ID NO: 1 or 2
have been mutated to cysteines and linked by a disulphide bridge.

In some embodiments of the present disclosure, the MHC class I molecule comprises or consists of at least amino acids 1-276 of the amino acid sequence according to SEQ ID NO: 1 or 2, or a variant of said MHC class I molecule having at least 85% sequence homology or identity thereto, such as at least 86% sequence homology or identity thereto, such as at least 87% sequence homology or identity thereto, such as at least 88% sequence homology or identity thereto, such as at least 89% sequence homology or identity thereto, such as at least 90% sequence homology or identity thereto, such as at least 91% sequence homology or identity thereto, such as at least 92% sequence homology or identity thereto, such as at least 93% sequence homology or identity thereto, such as at least 94% sequence homology or identity thereto, such as at least 95% sequence homology or identity thereto, such as at least 96% sequence homology or identity thereto, such as at least 97% sequence homology or identity thereto, such as at least 98% sequence homology or identity thereto, or such as at least 99% sequence homology or identity thereto,
with the proviso that the amino acids corresponding to positions
   ii) 12 and 94 of SEQ ID NO: 1 or 2,
   iii) 23 and 37 of SEQ ID NO: 1 or 2,
   iv) 115 and 125 of SEQ ID NO: 1 or 2
have been mutated to cysteines and linked by a disulphide bridge.

In some embodiments of the present disclosure, the MHC class I molecule comprises or consists of at least amino acids 1-276 of the amino acid sequence according to SEQ ID NO: 1 or 2, or a variant of said MHC class I molecule having at least 85% sequence homology or identity thereto, such as at least 86% sequence homology or identity thereto, such as at least 87% sequence homology or identity thereto, such as at least 88% sequence homology or identity thereto, such as at least 89% sequence homology or identity thereto, such as at least 90% sequence homology or identity thereto, such as at least 91% sequence homology or identity thereto, such as at least 92% sequence homology or identity thereto, such as at least 93% sequence homology or identity thereto, such as at least 94% sequence homology or identity thereto, such as at least 95% sequence homology or identity thereto, such as at least 96% sequence homology or identity thereto, such as at least 97% sequence homology or identity thereto, such as at least 98% sequence homology or identity thereto, or such as at least 99% sequence homology or identity thereto,
with the proviso that the amino acids corresponding to positions
   i) 4 and 102 of SEQ ID NO: 1 or 2, and
   ii) 12 and 94 of SEQ ID NO: 1 or 2, and
   iii) 23 and 37 of SEQ ID NO: 1 or 2, and
   iv) 115 and 125 of SEQ ID NO: 1 or 2
have been mutated to cysteines and linked by a disulphide bridge.

In some embodiments of the present disclosure, the MHC class I molecule comprises or consists of at least amino acids 115-276 of the amino acid sequence according to SEQ ID NO: 1 or 2, or a variant of said MHC class I molecule having at least 85% sequence homology or identity thereto, such as at least 86% sequence homology or identity thereto, such as at least 87% sequence homology or identity thereto, such as at least 88% sequence homology or identity thereto, such as at least 89% sequence homology or identity thereto, such as at least 90% sequence homology or identity thereto, such as at least 91% sequence homology or identity thereto, such as at least 92% sequence homology or identity thereto, such as at least 93% sequence homology or identity thereto, such as at least 94% sequence homology or identity thereto, such as at least 95% sequence homology or identity thereto, such as at least 96% sequence homology or identity thereto, such as at least 97% sequence homology or identity thereto, such as at least 98% sequence homology or identity thereto, or such as at least 99% sequence homology or identity thereto,
with the proviso that the amino acids corresponding to positions
   iv) 115 and 125 of SEQ ID NO: 1 or 2
have been mutated to cysteines and linked by a disulphide bridge.

In some embodiments of the present disclosure, the MHC class I molecule comprises or consists of at least amino acids 100-276 of the amino acid sequence according to SEQ ID NO: 1 or 2, such as at least amino acids 80-276 of the amino acid sequence according to SEQ ID NO: 1 or 2, such as at least amino acids 60-276 of the amino acid sequence according to SEQ ID NO: 1 or 2, such as at least amino acids 40-276 of the amino acid sequence according to SEQ ID NO: 1 or 2, such as at least amino acids 20-276 of the amino acid sequence according to SEQ ID NO: 1 or 2, or a variant of said MHC class I molecule having at least 85% sequence homology or identity thereto, such as at least 86% sequence homology or identity thereto, such as at least 87% sequence homology or identity thereto, such as at least 88% sequence homology or identity thereto, such as at least 89% sequence homology or identity thereto, such as at least 90% sequence homology or identity thereto, such as at least 91% sequence homology or identity thereto, such as at least 92% sequence homology or identity thereto, such as at least 93% sequence homology or identity thereto, such as at least 94% sequence homology or identity thereto, such as at least 95% sequence homology or identity thereto, such as at least 96% sequence homology or identity thereto, such as at least 97% sequence homology or identity thereto, such as at least 98% sequence homology or identity thereto, or such as at least 99% sequence homology or identity thereto,
with the proviso that the amino acids corresponding to positions
   iii) 23 and 37 of SEQ ID NO: 1 or 2, and/or
   iv) 115 and 125 of SEQ ID NO: 1 or 2
have been mutated to cysteines and linked by a disulphide bridge.

In some embodiments of the present disclosure, the MHC class I molecule comprises or consists of at least amino acids 100-276 of the amino acid sequence according to SEQ ID NO: 1 or 2, such as at least amino acids 80-276 of the amino acid sequence according to SEQ ID NO: 1 or 2, such as at least amino acids 60-276 of the amino acid sequence according to SEQ ID NO: 1 or 2, such as at least amino acids 40-276 of the amino acid sequence according to SEQ ID NO: 1 or 2, such as at least amino acids 20-276 of the amino acid sequence according to SEQ ID NO: 1 or 2, or a variant of said MHC class I molecule having at least 85% sequence homology or identity thereto, such as at least 86% sequence homology or identity thereto, such as at least 87% sequence homology or identity thereto, such as at least 88% sequence homology or identity thereto, such as at least 89% sequence homology or identity thereto, such as at least 90% sequence homology or identity thereto, such as at least 91% sequence homology or identity thereto, such as at least 92% sequence homology or identity thereto, such as at least 93% sequence homology or identity thereto, such as at least 94% sequence homology or identity thereto, such as at least 95% sequence homology or identity thereto, such as at least 96% sequence homology or identity thereto, such as at least 97% sequence homology or identity thereto, such as at least 98% sequence homology or identity thereto, or such as at least 99% sequence homology or identity thereto,
with the proviso that the amino acids corresponding to positions
   iv) 115 and 125 of SEQ ID NO: 1 or 2
have been mutated to cysteines and linked by a disulphide bridge.

In some embodiments of the present disclosure, the MHC class I molecule comprises or consists of at least amino acids 20-276 of the amino acid sequence according to SEQ ID NO: 1 or 2, or a variant of said MHC class I molecule having at least 85% sequence homology or identity thereto, such as at least 86% sequence homology or identity thereto, such as at least 87% sequence homology or identity thereto, such as at least 88% sequence homology or identity thereto, such as at least 89% sequence homology or identity thereto, such as at least 90% sequence homology or identity thereto, such as at least 91% sequence homology or identity thereto, such as at least 92% sequence homology or identity thereto, such as at least 93% sequence homology or identity thereto, such as at least 94% sequence homology or identity thereto, such as at least 95% sequence homology or identity thereto, such as at least 96% sequence homology or identity thereto, such as at least 97% sequence homology or identity thereto, such as at least 98% sequence homology or identity thereto, or such as at least 99% sequence homology or identity thereto,
with the proviso that the amino acids corresponding to positions
   iii) 23 and 37 of SEQ ID NO: 1 or 2, and
   iv) 115 and 125 of SEQ ID NO: 1 or 2
have been mutated to cysteines and linked by a disulphide bridge.

In some embodiments of the present disclosure, the MHC class I molecule comprises or consists of at the most amino acids 115-276 of the amino acid sequence according to SEQ ID NO: 1 or 2, or a variant of said MHC class I molecule having at least 85% sequence homology or identity thereto, such as at least 86% sequence homology or identity thereto, such as at least 87% sequence homology or identity thereto, such as at least 88% sequence homology or identity thereto, such as at least 89% sequence homology or identity thereto, such as at least 90% sequence homology or identity thereto, such as at least 91% sequence homology or identity thereto, such as at least 92% sequence homology or identity thereto, such as at least 93% sequence homology or identity thereto, such as at least 94% sequence homology or identity thereto, such as at least 95% sequence homology or identity thereto, such as at least 96% sequence homology or identity thereto, such as at least 97% sequence homology or identity thereto, such as at least 98% sequence homology or identity thereto, or such as at least 99% sequence homology or identity thereto,
with the proviso that the amino acids corresponding to positions
   iv) 115 and 125 of SEQ ID NO: 1 or 2
have been mutated to cysteines and linked by a disulphide bridge.

In some embodiments of the present disclosure, the MHC class I molecule comprises or consists of at the most amino acids 100-276 of the amino acid sequence according to SEQ ID NO: 1 or 2, such as at the most amino acids 80-276 of the amino acid sequence according to SEQ ID NO: 1 or 2, such as at the most amino acids 60-276 of the amino acid sequence according to SEQ ID NO: 1 or 2, such as at the most amino acids 40-276 of the amino acid sequence according to SEQ ID NO: 1 or 2, such as at the most amino acids 20-276 of the amino acid sequence according to SEQ ID NO: 1 or 2, or a variant of said MHC class I molecule having at least 85% sequence homology or identity thereto, such as at least 86% sequence homology or identity thereto, such as at least 87% sequence homology or identity thereto, such as at least 88% sequence homology or identity thereto, such as at least 89% sequence homology or identity thereto, such as at least 90% sequence homology or identity thereto, such as at least 91% sequence homology or identity thereto, such as at least 92% sequence homology or identity thereto, such as at least 93% sequence homology or identity thereto, such as at least 94% sequence homology or identity thereto, such as at least 95% sequence homology or identity thereto, such as at least 96% sequence homology or identity thereto, such as at least 97% sequence homology or identity thereto, such as at least 98% sequence homology or identity thereto, or such as at least 99% sequence homology or identity thereto,
with the proviso that the amino acids corresponding to positions
   iii) 23 and 37 of SEQ ID NO: 1 or 2, and/or
   iv) 115 and 125 of SEQ ID NO: 1 or 2
have been mutated to cysteines and linked by a disulphide bridge.

In some embodiments of the present disclosure, the MHC class I molecule comprises or consists of at the most amino acids 20-276 of the amino acid sequence according to SEQ ID NO: 1 or 2, or a variant of said MHC class I molecule having at least 85% sequence homology or identity thereto, such as at least 86% sequence homology or identity thereto, such as at least 87% sequence homology or identity thereto, such as at least 88% sequence homology or identity thereto, such as at least 89% sequence homology or identity thereto, such as at least 90% sequence homology or identity thereto, such as at least 91% sequence homology or identity thereto, such as at least 92% sequence homology or identity thereto, such as at least 93% sequence homology or identity thereto, such as at least 94% sequence homology or identity thereto, such as at least 95% sequence homology or identity thereto, such as at least 96% sequence homology or identity thereto, such as at least 97% sequence homology or identity thereto, such as at least 98% sequence homology or identity thereto, or such as at least 99% sequence homology or identity thereto,
with the proviso that the amino acids corresponding to positions
   iii) 23 and 37 of SEQ ID NO: 1 or 2, and
   iv) 115 and 125 of SEQ ID NO: 1 or 2
have been mutated to cysteines and linked by a disulphide bridge.

In some embodiments of the present disclosure, the MHC class I molecule comprises or consists of at least amino acids 91-276 of the amino acid sequence according to SEQ ID NO: 1 or 2, or a variant of said MHC class I molecule having at least 85% sequence homology or identity thereto, such as at least 86% sequence homology or identity thereto, such as at least 87% sequence homology or identity thereto, such as at least 88% sequence homology or identity thereto, such as at least 89% sequence homology or identity thereto, such as at least 90% sequence homology or identity thereto, such as at least 91% sequence homology or identity thereto, such as at least 92% sequence homology or identity thereto, such as at least 93% sequence homology or identity thereto, such as at least 94% sequence homology or identity thereto, such as at least 95% sequence homology or identity thereto, such as at least 96% sequence homology or identity thereto, such as at least 97% sequence homology or identity thereto, such as at least 98% sequence homology or identity thereto, or such as at least 99% sequence homology or identity thereto,
with the proviso that the amino acids corresponding to positions
   iv) 115 and 125 of SEQ ID NO: 1 or 2
have been mutated to cysteines and linked by a disulphide bridge.

In some embodiments of the present disclosure, the MHC class I molecule comprises or consists of the amino acid sequence as set forth in SEQ ID NO: 3 or SEQ ID NO: 4, or comprises or consists of a variant of said MHC class I molecule having at least 85% sequence homology or identity thereto, such as at least 86% sequence homology or identity thereto, such as at least 87% sequence homology or identity thereto, such as at least 88% sequence homology or identity thereto, such as at least 89% sequence homology or identity thereto, such as at least 90% sequence homology or identity thereto, such as at least 91% sequence homology or identity thereto, such as at least 92% sequence homology or identity thereto, such as at least 93% sequence homology or identity thereto, such as at least 94% sequence homology or identity thereto, such as at least 95% sequence homology or identity thereto, such as at least 96% sequence homology or identity thereto, such as at least 97% sequence homology or identity thereto, such as at least 98% sequence homology or identity thereto, or such as at least 99% sequence homology or identity thereto,
with the proviso that the amino acids corresponding to positions 4 and 102 of SEQ ID NO: 3 or SEQ ID NO: 4 are cysteines and linked by a disulphide bridge.

In some embodiments of the present disclosure, the MHC class I molecule comprises or consists of the amino acid sequence as set forth in SEQ ID NO: 5 or SEQ ID NO: 6, or comprises or consists of a variant of said MHC class I molecule having at least 85% sequence homology or identity thereto, such as at least 86% sequence homology or identity thereto, such as at least 87% sequence homology or identity thereto, such as at least 88% sequence homology or identity thereto, such as at least 89% sequence homology or identity thereto, such as at least 90% sequence homology or identity thereto, such as at least 91% sequence homology or identity thereto, such as at least 92% sequence homology or identity thereto, such as at least 93% sequence homology or identity thereto, such as at least 94% sequence homology or identity thereto, such as at least 95% sequence homology or identity thereto, such as at least 96% sequence homology or identity thereto, such as at least 97% sequence homology or identity thereto, such as at least 98% sequence homology or identity thereto, or such as at least 99% sequence homology or identity thereto,
with the proviso that the amino acids corresponding to positions 12 and 94 of SEQ ID NO: 5 or SEQ ID NO: 6 are cysteines and linked by a disulphide bridge.

In some embodiments of the present disclosure, the MHC class I molecule comprises or consists of the amino acid sequence as set forth in SEQ ID NO: 7 or SEQ ID NO: 8, or comprises or consists of a variant of said MHC class I molecule having at least 85% sequence homology or identity thereto, such as at least 86% sequence homology or identity thereto, such as at least 87% sequence homology or identity thereto, such as at least 88% sequence homology or identity thereto, such as at least 89% sequence homology or identity thereto, such as at least 90% sequence homology or identity thereto, such as at least 91% sequence homology or identity thereto, such as at least 92% sequence homology or identity thereto, such as at least 93% sequence homology or identity thereto, such as at least 94% sequence homology or identity thereto, such as at least 95% sequence homology or identity thereto, such as at least 96% sequence homology or identity thereto, such as at least 97% sequence homology or identity thereto, such as at least 98% sequence homology or identity thereto, or such as at least 99% sequence homology or identity thereto,
with the proviso that the amino acids corresponding to positions 115 and 125 of SEQ ID NO: 7 or SEQ ID NO: 8 are cysteines and linked by a disulphide bridge.

In some embodiments of the present disclosure, the MHC class I molecule comprises or consists of the amino acid sequence as set forth in SEQ ID NO: 9 or SEQ ID NO: 10, or comprises or consists of a variant of said MHC class I molecule having at least 85% sequence homology or identity thereto, such as at least 86% sequence homology or identity thereto, such as at least 87% sequence homology or identity thereto, such as at least 88% sequence homology or identity thereto, such as at least 89% sequence homology or identity thereto, such as at least 90% sequence homology or identity thereto, such as at least 91% sequence homology or identity thereto, such as at least 92% sequence homology or identity thereto, such as at least 93% sequence homology or identity thereto, such as at least 94% sequence homology or identity thereto, such as at least 95% sequence homology or identity thereto, such as at least 96% sequence homology or identity thereto, such as at least 97% sequence homology or identity thereto, such as at least 98% sequence homology or identity thereto, or such as at least 99% sequence homology or identity thereto,
with the proviso that the amino acids corresponding to positions 23 and 37 of SEQ ID NO: 9 or SEQ ID NO: 10 are cysteines and linked by a disulphide bridge.

In some embodiments the present disclosure provides stable MHC class I molecules which comprises at least two cysteine mutations linked by a disulphide bridge. In some embodiments the present disclosure provides stable MHC class I molecules which comprises at least two pairs of cysteine mutations linked by a disulphide bridge. In some embodiments the present disclosure provides stable MHC class I molecules which comprises at least three pairs of cysteine mutations linked by a disulphide bridge. In some embodiments the present disclosure provides stable MHC class I molecules which comprises at least four pairs of cysteine mutations linked by a disulphide bridge.

In some embodiments of the present disclosure, the MHC class I molecule according to the present disclosure comprises no more than two cysteine mutations linked by a disulphide bridge.

In some embodiments, the MHC class I molecule according to the present disclosure
i. has increased stability, such as an increased melting temperature, such as increased stability compared to a corresponding wild type MHC class I molecule of identical sequence except not comprising said cysteine mutations linked by a disulphide bridge, and/or
ii. comprises a refolding test peptide in its peptide-binding groove and has an increased refolding yield compared to a corresponding wild type MHC class I molecule of identical sequence except not comprising said cysteine mutations linked by a disulphide bridge, said corresponding wild type MHC class I molecule also comprising said refolding test peptide in its peptide-binding groove.

In some embodiments, the MHC class I molecule according to the present disclosure
i. has increased stability, such as an increased melting temperature, such as increased stability compared to a corresponding wild type MHC class I molecule of identical sequence except not comprising said cysteine mutations linked by a disulphide bridge, and
ii. comprises a refolding test peptide in its peptide-binding groove and has an increased refolding yield compared to a corresponding wild type MHC class I molecule of identical sequence except not comprising said cysteine mutations linked by a disulphide bridge, said corresponding wild type MHC class I molecule also comprising said refolding test peptide in its peptide-binding groove.

In some embodiments the MHC class I molecule according to the present disclosure comprising at least two cysteine mutations linked by a disulphide bridge are stable, such as has improved stability.

In some embodiments of the present disclosure, the MHC class I molecule according to the present disclosure comprising at least two cysteine mutations linked by a disulphide bridge has increased stability; such as increased melting temperature; such as increased stability compared to a corresponding wild type MHC class I molecule of identical sequence except not comprising said cysteine mutations linked by a disulphide bridge.

In some embodiments the MHC class I molecule of the present disclosure, has an increased melting temperature of at least 1.00° C., such as at least 1.25° C., such as at least 1.50° C., such as at least 1.75° C., such as at least 2.0° C., such as at least 2.25° C., such as at least 2.5° C., such as at least 2.75° C., such as at least 3.00° C., such as at least 3.5° C., such as at least 4.0° C., such as at least 4.5° C., such as at least 5.0° C., such as at least 5.5° C. or such as at least 6.0° C., compared to a corresponding wild type MHC class I molecule of identical sequence except not comprising said cysteine mutations linked by a disulphide bridge.

In some embodiments, the MHC class I molecule of the present disclosure has a melting temperature of at least 49.0° C. In some embodiments the MHC class I molecule of the present disclosure has a melting temperature of at least 49.5° C. In some embodiments, the MHC class I molecule of the present disclosure has a melting temperature of at least 50.0° C. In some embodiments, the MHC class I molecule of the present disclosure has a melting temperature of at least 50.5° C. In some embodiments the MHC class I molecule of the present disclosure has a melting temperature of at least 51.0° C. In some embodiments, the MHC class I molecule of the present disclosure has a melting temperature of at least 51.5° C. In some embodiments, the MHC class I molecule of the present disclosure has a melting temperature of at least 52° C. In some embodiments, the MHC class I molecule of the present disclosure has a melting temperature of at least 52.5° C. In some embodiments, the MHC class I molecule of the present disclosure has a melting temperature of at least 53.0° C. In some embodiments, the MHC class I molecule of the present disclosure has a melting temperature of at least 53.5° C. In some embodiments, the MHC class I molecule of the present disclosure has a melting temperature of at least 54.0° C. In some embodiments, the MHC class I molecule of the present disclosure has a melting temperature of at least 54.5° C. In some embodiments, the MHC class I molecule of the present disclosure has a melting temperature of at least 55.0° C. In some embodiments, the MHC class I molecule of the present disclosure has a melting temperature of at least 55.5° C. In some embodiments, the MHC class I molecule of the present disclosure has a melting temperature of at least 56.0° C.

In some embodiments, the MHC class I molecule of the present disclosure has a melting temperature of about 49.0° C., such as about 49.5° C., such as about 50.0° C., such as about 50.5° C., such as about 51.0° C., such as 51.5° C., such as about 52.0° C., such as 52.5° C., such as about 53.0° C., such as about 53.5° C., such as about 54.0° C., such as about 54.5° C., such as about 55.0° C., such as about 55.5° C., such as about 56.0° C., such as about 56.5° C., such as about 57.0° C., such as about 57.5° C., such as about 58.0° C., such as about 58.5° C., such as about 59.0° C., such as about 59.5° C., such as about 60.0° C.

In some embodiments, the MHC class I molecule of the present disclosure has a melting temperature of about 49.0° C. to about 49.5° C., such as about 49.5° C. to about 50.0° C., such as about 50.0° C. to about 50.5° C., such as about 50.5° C. to about 51.0° C., such as about 51.0° C. to about 51.5° C., such as about 51.5° C. to about 52.0° C., such as about 52.0° C. to about 52.5° C., such as about 52.5° C. to about 53.0° C., such as about 53.0° C. to about 53.5° C., such as about 53.5° C. to about 54.0° C., such as about 54.0° C. to about 54.5° C., such as about 54.5° C. to about 55.0° C., such as about 55.0° C. to about 55.5° C., such as about 55.5° C. to about 56.0° C., such as about 56.0° C. to about 56.5° C., such as about 56.5° C. to about 57.0° C., such as about 57.0° C. to about 57.5° C., such as about 57.5° C. to about 58.0° C., such as about 58.0° C. to about 58.5° C., such as about 58.5° C. to about 59.0° C., such as about 59.0° C. to about 59.5° C., such as about 59.5° C. to about 60.0° C., such as about 60.0° C. to about 61.0° C., such as about 61.0° C. to about 62.0° C., such as about 62.0° C. to about 63.0° C., such as about 63.0° C. to about 64.0° C., such as about 64.0° C. to about 65.0° C., such as about 65.0° C. to about 66.0° C., such as about 66.0° C. to about 67.0° C., such as about 67.0° C. to about 68.0° C., such as about 68.0° C. to about 69.0° C., such as about 69.0° C. to about 70.0° C.

In some embodiments of the present disclosure, said melting temperature is measured by quantitative polymerase chain reaction (qPCR). In some embodiments of the present disclosure, said melting temperature is measured by nano differential scanning fluorimetry (NanoDSF).

In some embodiments, the MHC class I molecule of the present disclosure has an increased melting temperature compared to a corresponding wild type MHC class I molecule of identical sequence except not comprising said cysteine mutations linked by a disulphide bridge, wherein said MHC Class I molecule comprises or consists of the amino acid sequence as set forth in SEQ ID NO: 3 or SEQ ID NO: 4, or comprises or consists of a variant of said MHC class I molecule having at least 85% sequence homology or identity thereto, such as at least 86% sequence homology or identity thereto, such as at least 87% sequence homology or identity thereto, such as at least 88% sequence homology or identity thereto, such as at least 89% sequence homology or identity thereto, such as at least 90% sequence homology or identity thereto, such as at least 91% sequence homology or identity thereto, such as at least 92% sequence homology or identity thereto, such as at least 93% sequence homology or identity thereto, such as at least 94% sequence homology or identity thereto, such as at least 95% sequence homology or identity thereto, such as at least 96% sequence homology or identity thereto, such as at least 97% sequence homology or identity thereto, such as at least 98% sequence homology or identity thereto, or such as at least 99% sequence homology or identity thereto,
with the proviso that the amino acids corresponding to positions 4 and 102 of SEQ ID NO: 3 or 4 are cysteines and linked by a disulphide bridge.

In some embodiments, the MHC class I molecule of the present disclosure has an increased melting temperature compared to a corresponding wild type MHC class I molecule of identical sequence except not comprising said cysteine mutations linked by a disulphide bridge, wherein said MHC Class I molecule comprises or consists of the amino acid sequence as set forth in SEQ ID NO: 5 or SEQ ID NO: 6, or comprises or consists of a variant of said MHC class I molecule having at least 85% sequence homology or identity thereto, such as at least 86% sequence homology or identity thereto, such as at least 87% sequence homology or identity thereto, such as at least 88% sequence homology or identity thereto, such as at least 89% sequence homology or identity thereto, such as at least 90% sequence homology or identity thereto, such as at least 91% sequence homology or identity thereto, such as at least 92% sequence homology or identity thereto, such as at least 93% sequence homology or identity thereto, such as at least 94% sequence homology or identity thereto, such as at least 95% sequence homology or identity thereto, such as at least 96% sequence homology or identity thereto, such as at least 97% sequence homology or identity thereto, such as at least 98% sequence homology or identity thereto, or such as at least 99% sequence homology or identity thereto,
with the proviso that the amino acids corresponding to positions 12 and 94 of SEQ ID NO: 5 or 6 are cysteines and linked by a disulphide bridge.

In some embodiments, the MHC class I molecule of the present disclosure has an increased melting temperature compared to a corresponding wild type MHC class I molecule of identical sequence except not comprising said cysteine mutations linked by a disulphide bridge, wherein said MHC Class I molecule comprises or consists of the amino acid sequence as set forth in SEQ ID NO: 7 or SEQ ID NO: 8, or comprises or consists of a variant of said MHC class I molecule having at least 85% sequence homology or identity thereto, such as at least 86% sequence homology or identity thereto, such as at least 87% sequence homology or identity thereto, such as at least 88% sequence homology or identity thereto, such as at least 89% sequence homology or identity thereto, such as at least 90% sequence homology or identity thereto, such as at least 91% sequence homology or identity thereto, such as at least 92% sequence homology or identity thereto, such as at least 93% sequence homology or identity thereto, such as at least 94% sequence homology or identity thereto, such as at least 95% sequence homology or identity thereto, such as at least 96% sequence homology or identity thereto, such as at least 97% sequence homology or identity thereto, such as at least 98% sequence homology or identity thereto, or such as at least 99% sequence homology or identity thereto,
with the proviso that the amino acids corresponding to positions 115 and 125 of SEQ ID NO: 7 or 8 are cysteines and linked by a disulphide bridge.

In some embodiments, the MHC class I molecule of the present disclosure comprises or consists of the amino acid sequence as set forth in SEQ ID NO: 3 or SEQ ID NO: 4, or comprises or consists of a variant of said MHC class I molecule having at least 85% sequence homology or identity thereto, such as at least 86% sequence homology or identity thereto, such as at least 87% sequence homology or identity thereto, such as at least 88% sequence homology or identity thereto, such as at least 89% sequence homology or identity thereto, such as at least 90% sequence homology or identity thereto, such as at least 91% sequence homology or identity thereto, such as at least 92% sequence homology or identity thereto, such as at least 93% sequence homology or identity thereto, such as at least 94% sequence homology or identity thereto, such as at least 95% sequence homology or identity thereto, such as at least 96% sequence homology or identity thereto, such as at least 97% sequence homology or identity thereto, such as at least 98% sequence homology or identity thereto, or such as at least 99% sequence homology or identity thereto, with the proviso that the amino acids corresponding to positions 4 and 102 of SEQ ID NO: 3 or 4 are cysteines and linked by a disulphide bridge;
wherein said MHC class I molecule has a melting temperature of at least 49.0° C., such as at least 49.5° C., such as at least 50.0° C., such as at least 50.5° C., such as at least 51.0° C., such as at least 51.5° C., such as at least 52.0° C., such as at least 52.5° C., such as at least 53.0° C., such as at least 53.5° C., such as at least 54.0° C., such as at least 54.5° C., such as at least 55.0° C., such as at least 55.5° C., such as at least 56.0° C.; or
wherein said MHC class I molecule has a melting temperature of about 49.0° C., such as about 49.5° C., such as about 50.0° C., such as about 50.5° C., such as about 51.0° C., such as 51.5° C., such as about 52.0° C., such as 52.5° C., such as about 53.0° C., such as about 53.5° C., such as about 54.0° C., such as about 54.5° C., such as about 55.0° C., such as about 55.5° C., such as about 56.0° C., such as about 56.5° C., such as about 57.0° C., such as about 57.5° C., such as about 58.0° C., such as about 58.5° C., such as about 59.0° C., such as about 59.5° C., such as about 60.0° C.; or
wherein said MHC class I molecule has a melting temperature of about 49.0° C. to about 49.5° C., such as about 49.5° C. to about 50.0° C., such as about 50.0° C. to about 50.5° C., such as about 50.5° C. to about 51.0° C., such as about 51.0° C. to about 51.5° C., such as about 51.5° C. to about 52.0° C., such as about 52.0° C. to about 52.5° C., such as about 52.5° C. to about 53.0° C., such as about 53.0° C. to about 53.5° C., such as about 53.5° C. to about 54.0° C., such as about 54.0° C. to about 54.5° C., such as about 54.5° C. to about 55.0° C., such as about 55.0° C. to about 55.5° C., such as about 55.5° C. to about 56.0° C., such as about 56.0° C. to about 56.5° C., such as about 56.5° C. to about 57.0° C., such as about 57.0° C. to about 57.5° C., such as about 57.5° C. to about 58.0° C., such as about 58.0° C. to about 58.5° C., such as about 58.5° C. to about 59.0° C., such as about 59.0° C. to about 59.5° C., such as about 59.5° C. to about 60.0° C., such as about 60.0° C. to about 61.0° C., such as about 61.0° C. to about 62.0° C., such as about 62.0° C. to about 63.0° C., such as about 63.0° C. to about 64.0° C., such as about 64.0° C. to about 65.0° C., such as about 65.0° C. to about 66.0° C., such as about 66.0° C. to about 67.0° C., such as about 67.0° C. to about 68.0° C., such as about 68.0° C. to about 69.0° C., such as about 69.0° C. to about 70.0° C.

In some embodiments, the MHC class I molecule of the present disclosure comprises or consists of the amino acid sequence as set forth in SEQ ID NO: 5 or SEQ ID NO: 6, or comprises or consists of a variant of said MHC class I molecule having at least 85% sequence homology or identity thereto, such as at least 86% sequence homology or identity thereto, such as at least 87% sequence homology or identity thereto, such as at least 88% sequence homology or identity thereto, such as at least 89% sequence homology or identity thereto, such as at least 90% sequence homology or identity thereto, such as at least 91% sequence homology or identity thereto, such as at least 92% sequence homology or identity thereto, such as at least 93% sequence homology or identity thereto, such as at least 94% sequence homology or identity thereto, such as at least 95% sequence homology or identity thereto, such as at least 96% sequence homology or identity thereto, such as at least 97% sequence homology or identity thereto, such as at least 98% sequence homology or identity thereto, or such as at least 99% sequence homology or identity thereto, with the proviso that the amino acids corresponding to positions 12 and 94 of SEQ ID NO: 5 or 6 are cysteines and linked by a disulphide bridge;
wherein said MHC class I molecule has a melting temperature of at least 49.0° C., such as at least 49.5° C., such as at least 50.0° C., such as at least 50.5° C., such as at least 51.0° C., such as at least 51.5° C., such as at least 52.0° C., such as at least 52.5° C., such as at least 53.0° C., such as at least 53.5° C., such as at least 54.0° C., such as at least 54.5° C., such as at least 55.0° C., such as at least 55.5° C., such as at least 56.0° C.; or
wherein said MHC class I molecule has a melting temperature of about 49.0° C., such as about 49.5° C., such as about 50.0° C., such as about 50.5° C., such as about 51.0° C., such as 51.5° C., such as about 52.0° C., such as 52.5° C., such as about 53.0° C., such as about 53.5° C., such as about 54.0° C., such as about 54.5° C., such as about 55.0° C., such as about 55.5° C., such as about 56.0° C., such as about 56.5° C., such as about 57.0° C., such as about 57.5° C., such as about 58.0° C., such as about 58.5° C., such as about 59.0° C., such as about 59.5° C., such as about 60.0° C.; or wherein said MHC class I molecule has a melting temperature of about 49.0° C. to about 49.5° C., such as about 49.5° C. to about 50.0° C., such as about 50.0° C. to about 50.5° C., such as about 50.5° C. to about 51.0° C., such as about 51.0° C. to about 51.5° C., such as about 51.5° C. to about 52.0° C., such as about 52.0° C. to about 52.5° C., such as about 52.5° C. to about 53.0° C., such as about 53.0° C. to about 53.5° C., such as about 53.5° C. to about 54.0° C., such as about 54.0° C. to about 54.5° C., such as about 54.5° C. to about 55.0° C., such as about 55.0° C. to about 55.5° C., such as about 55.5° C. to about 56.0° C., such as about 56.0° C. to about 56.5° C., such as about 56.5° C. to about 57.0° C., such as about 57.0° C. to about 57.5° C., such as about 57.5° C. to about 58.0° C., such as about 58.0° C. to about 58.5° C., such as about 58.5° C. to about 59.0° C., such as about 59.0° C. to about 59.5° C., such as about 59.5° C. to about 60.0° C., such as about 60.0° C. to about 61.0° C., such as about 61.0° C. to about 62.0° C., such as about 62.0° C. to about 63.0° C., such as about 63.0° C. to about 64.0° C., such as about 64.0° C. to about 65.0° C., such as about 65.0° C. to about 66.0° C., such as about 66.0° C. to about 67.0° C., such as about 67.0° C. to about 68.0° C., such as about 68.0° C. to about 69.0° C., such as about 69.0° C. to about 70.0° C.

In some embodiments, the MHC class I molecule of the present disclosure comprises or consists of the amino acid sequence as set forth in SEQ ID NO: 7 or SEQ ID NO: 8, or comprises or consists of a variant of said MHC class I molecule having at least 85% sequence homology or identity thereto, such as at least 86% sequence homology or identity thereto, such as at least 87% sequence homology or identity thereto, such as at least 88% sequence homology or identity thereto, such as at least 89% sequence homology or identity thereto, such as at least 90% sequence homology or identity thereto, such as at least 91% sequence homology or identity thereto, such as at least 92% sequence homology or identity thereto, such as at least 93% sequence homology or identity thereto, such as at least 94% sequence homology or identity thereto, such as at least 95% sequence homology or identity thereto, such as at least 96% sequence homology or identity thereto, such as at least 97% sequence homology or identity thereto, such as at least 98% sequence homology or identity thereto, or such as at least 99% sequence homology or identity thereto, with the proviso that the amino acids corresponding to positions 115 and 125 of SEQ ID NO: 7 or 8 are cysteines and linked by a disulphide bridge,
wherein said MHC class I molecule has a melting temperature of at least 49.0° C., such as at least 49.5° C., such as at least 50.0° C., such as at least 50.5° C., such as at least 51.0° C., such as at least 51.5° C., such as at least 52.0° C., such as at least 52.5° C., such as at least 53.0° C., such as at least 53.5° C., such as at least 54.0° C., such as at least 54.5° C., such as at least 55.0° C., such as at least 55.5° C., such as at least 56.0° C.; or
wherein said MHC class I molecule has a melting temperature of about 49.0° C., such as about 49.5° C., such as about 50.0° C., such as about 50.5° C., such as about 51.0° C., such as 51.5° C., such as about 52.0° C., such as 52.5° C., such as about 53.0° C., such as about 53.5° C., such as about 54.0° C., such as about 54.5° C., such as about 55.0° C., such as about 55.5° C., such as about 56.0° C., such as about 56.5° C., such as about 57.0° C., such as about 57.5° C., such as about 58.0° C., such as about 58.5° C., such as about 59.0° C., such as about 59.5° C., such as about 60.0° C.; or wherein said MHC class I molecule has a melting temperature of about 49.0° C. to about 49.5° C., such as about 49.5° C. to about 50.0° C., such as about 50.0° C. to about 50.5° C., such as about 50.5° C. to about 51.0° C., such as about 51.0° C. to about 51.5° C., such as about 51.5° C. to about 52.0° C., such as about 52.0° C. to about 52.5° C., such as about 52.5° C. to about 53.0° C., such as about 53.0° C. to about 53.5° C., such as about 53.5° C. to about 54.0° C., such as about 54.0° C. to about 54.5° C., such as about 54.5° C. to about 55.0° C., such as about 55.0° C. to about 55.5° C., such as about 55.5° C. to about 56.0° C., such as about 56.0° C. to about 56.5° C., such as about 56.5° C. to about 57.0° C., such as about 57.0° C. to about 57.5° C., such as about 57.5° C. to about 58.0° C., such as about 58.0° C. to about 58.5° C., such as about 58.5° C. to about 59.0° C., such as about 59.0° C. to about 59.5° C., such as about 59.5° C. to about 60.0° C., such as about 60.0° C. to about 61.0° C., such as about 61.0° C. to about 62.0° C., such as about 62.0° C. to about 63.0° C., such as about 63.0° C. to about 64.0° C., such as about 64.0° C. to about 65.0° C., such as about 65.0° C. to about 66.0° C., such as about 66.0° C. to about 67.0° C., such as about 67.0° C. to about 68.0° C., such as about 68.0° C. to about 69.0° C., such as about 69.0° C. to about 70.0° C.

In some embodiments the MHC class I molecule according to the present disclosure comprising at least two cysteine mutations linked by a disulphide bridge has an improved refolding yield.

In some embodiments, the MHC class I molecule according to the present disclosure comprising at least two cysteine mutations linked by a disulphide bridge has increased refolding yield compared to a corresponding wild type MHC class I molecule of identical sequence except not comprising said cysteine mutations linked by a disulphide bridge.

The refolding yield of the MHC class I molecule according to the present disclosure and said corresponding wild type MHC class I molecule of identical sequence except not comprising said cysteine mutations linked by a disulphide bridge may preferably be determined with each of these two molecules comprising a refolding test peptide in the binding grove of the MHC class I molecule.

Thus, in some embodiments the MHC class I molecule of the present disclosure comprises a refolding test peptide in its peptide-binding groove and has an increased refolding yield compared to a corresponding wild type MHC class I molecule of identical sequence except not comprising said cysteine mutations linked by a disulphide bridge, said corresponding wild type MHC class I molecule also comprising said refolding test peptide in its peptide-binding groove.

Any peptide which is able to bind in the binding groove of the MHC class I may be used as a refolding test peptide. In some embodiments of the present disclosure, the refolding test peptide is inhA as set forth in SEQ ID NO: 12. In some embodiments of the present disclosure, the refolding test peptide is UL40 as set forth in SEQ ID NO: 13.

In particular, the refolding yield of the MHC class I molecule may be determined as disclosed in Example 2. Assuming that all HC added to a refolding mix can form MHC complexes with peptide and β₂m, the theoretical yield of a given refolding reaction can be calculated. The actual yield can be measured e.g. as the absorbance at 280 nm of the final, purified MHC complex solution. The A280 readouts can be transformed into a protein concentration and then a total yield. Comparing this actual yield with the theoretical yield gives the relative yield and thus the refolding yield as described herein.

In some embodiments the MHC class I molecule of the present disclosure has an increased refolding yield compared to a corresponding wild type MHC class I molecule of identical sequence except not comprising said cysteine mutations linked by a disulphide bridge, and comprises or consists of the amino acid sequence as set forth in SEQ ID NO: 3 or SEQ ID NO: 4, or comprises or consists of a variant of said MHC class I molecule having at least 85% sequence homology or identity thereto, such as at least 86% sequence homology or identity thereto, such as at least 87% sequence homology or identity thereto, such as at least 88% sequence homology or identity thereto, such as at least 89% sequence homology or identity thereto, such as at least 90% sequence homology or identity thereto, such as at least 91% sequence homology or identity thereto, such as at least 92% sequence homology or identity thereto, such as at least 93% sequence homology or identity thereto, such as at least 94% sequence homology or identity thereto, such as at least 95% sequence homology or identity thereto, such as at least 96% sequence homology or identity thereto, such as at least 97% sequence homology or identity thereto, such as at least 98% sequence homology or identity thereto, or such as at least 99% sequence homology or identity thereto,
with the proviso that the amino acids corresponding to positions 4 and 102 of SEQ ID NO: 3 or 4 are cysteines and linked by a disulphide bridge, and wherein said refolding test peptide is inhA as set forth in SEQ ID NO: 12.

In some embodiments of the present disclosure, the MHC class I molecule has an increased refolding yield compared to a corresponding wild type MHC class I molecule of identical sequence except not comprising said cysteine mutations linked by a disulphide bridge, and comprises or consists of the amino acid sequence as set forth in SEQ ID NO: 5 or SEQ ID NO: 6, or comprises or consists of a variant of said MHC class I molecule having at least 85% sequence homology or identity thereto, such as at least 86% sequence homology or identity thereto, such as at least 87% sequence homology or identity thereto, such as at least 88% sequence homology or identity thereto, such as at least 89% sequence homology or identity thereto, such as at least 90% sequence homology or identity thereto, such as at least 91% sequence homology or identity thereto, such as at least 92% sequence homology or identity thereto, such as at least 93% sequence homology or identity thereto, such as at least 94% sequence homology or identity thereto, such as at least 95% sequence homology or identity thereto, such as at least 96% sequence homology or identity thereto, such as at least 97% sequence homology or identity thereto, such as at least 98% sequence homology or identity thereto, or such as at least 99% sequence homology or identity thereto,
with the proviso that the amino acids corresponding to positions 12 and 94 of SEQ ID NO: 5 or 6 are cysteines and linked by a disulphide bridge, and wherein said refolding test peptide is inhA as set forth in SEQ ID NO: 12 or UL40 as set forth in SEQ ID NO: 13.

In some embodiments of the present disclosure, the MHC class I molecule has an increased refolding yield compared to a corresponding wild type MHC class I molecule of identical sequence except not comprising said cysteine mutations linked by a disulphide bridge, and comprises or consists of the amino acid sequence as set forth in SEQ ID NO: 7 or SEQ ID NO: 8, or comprises or consists of a variant of said MHC class I molecule having at least 85% sequence homology or identity thereto, such as at least 86% sequence homology or identity thereto, such as at least 87% sequence homology or identity thereto, such as at least 88% sequence homology or identity thereto, such as at least 89% sequence homology or identity thereto, such as at least 90% sequence homology or identity thereto, such as at least 91% sequence homology or identity thereto, such as at least 92% sequence homology or identity thereto, such as at least 93% sequence homology or identity thereto, such as at least 94% sequence homology or identity thereto, such as at least 95% sequence homology or identity thereto, such as at least 96% sequence homology or identity thereto, such as at least 97% sequence homology or identity thereto, such as at least 98% sequence homology or identity thereto, or such as at least 99% sequence homology or identity thereto,
with the proviso that the amino acids corresponding to positions 115 and 125 of SEQ ID NO: 7 or 8 are cysteines and linked by a disulphide bridge,
and wherein said refolding test peptide is inhA as set forth in SEQ ID NO: 12.

In some embodiments of the present disclosure, the MHC class I molecule has an increased refolding yield compared to a corresponding wild type MHC class I molecule of identical sequence except not comprising said cysteine mutations linked by a disulphide bridge, and comprises or consists of the amino acid sequence as set forth in SEQ ID NO: 9 or SEQ ID NO: 10, or comprises or consists of a variant of said MHC class I molecule having at least 85% sequence homology or identity thereto, such as at least 86% sequence homology or identity thereto, such as at least 87% sequence homology or identity thereto, such as at least 88% sequence homology or identity thereto, such as at least 89% sequence homology or identity thereto, such as at least 90% sequence homology or identity thereto, such as at least 91% sequence homology or identity thereto, such as at least 92% sequence homology or identity thereto, such as at least 93% sequence homology or identity thereto, such as at least 94% sequence homology or identity thereto, such as at least 95% sequence homology or identity thereto, such as at least 96% sequence homology or identity thereto, such as at least 97% sequence homology or identity thereto, such as at least 98% sequence homology or identity thereto, or such as at least 99% sequence homology or identity thereto,
with the proviso that the amino acids corresponding to positions 23 and 37 of SEQ ID NO: 9 or 10 are cysteines and linked by a disulphide bridge,
and wherein said refolding test peptide is inhA as set forth in SEQ ID NO: 12.

In some embodiments the MHC class I molecule of the present disclosure has an increased refolding yield of at least 125%, such as at least 130%, such as at least 135%, such as at least 140%, such as at least 145%, such as at least 150%, such as at least 155%, such as at least 160%, such as at least 165%, such as at least 170%, such as at least 175%, such as at least 180%, such as at least 185%, such as at least 190%, such as at least 195%, such as at least 200%, such as at least 205%, such as at least 210%, such as at least 215%, such as at least 220%, such as at least 225%, such as at least 230%, such as at least 235%, such as at least 240%, such as at least 245%, such as at least 250%, such as at least 255%, such as at least 260%, such as at least 265%, such as at least 270%, such as at least 275%, such as at least 280%, such as at least 285%, such as at least 290%, such as at least 295%, such as at least 300%, such as at least 305%, such as at least 310%, such as at least 315%, such as at least 320%, such as at least 325%, such as at least 330%, such as at least 335%, such as at least 340%, such as at least 345%, such as at least 350%, such as at least 355%, such as at least 360%, such as at least 365%, such as at least 370%, such as at least 375%, such as at least 380%, such as at least 385%, such as at least 390%, such as at least 395%, such as at least 400%, such as at least 405%, such as at least 410%, such as at least 415%, such as at least 420%, such as at least 425%, such as at least 430%, such as at least 435%, such as at least 440%, such as at least 445%, or such as at least 450% compared to said corresponding wild type MHC class I molecule.

In some embodiments the MHC class I molecule of the present disclosure has an increased refolding yield of about 125%, such as about 130%, such as about 135%, such as about 140%, such as about 145%, such as about 150%, such as about 155% such as about 160%, such as about 165%, such as about 170%, such as about 175% such as about 180%, such as about 185%, such as about 190%, such as about 195% such as about 200%, such as about 205%, such as about 210%, such as about 215% such as about 220%, such as about 225%, such as about 230%, such as about 235% such as about 240%, such as about 245%, such as about 250%, such as about 255% such as about 260%, such as about 265%, such as about 270%, such as about 275% such as about 280%, such as about 285%, such as about 290%, such as about 295% such as about 300%, such as about 305%, such as about 310%, such as about 315% such as about 320%, such as about 325%, such as about 330%, such as about 335% such as about 340%, such as about 345%, such as about 350%, such as about 355% such as about 360%, such as about 365%, such as about 370%, such as about 375% such as about 380%, such as about 385%, such as about 390%, such as about 395% such as about 400%, such as about 405%, such as about 410%, such as about 415% such as about 420%, such as about 425%, such as about 430%, such as about 435% such as about 440%, such as about 445%, or such as about 450% compared to said corresponding wild type MHC class I molecule.

In some embodiments the MHC class I molecule of the present disclosure has an increased refolding yield of about 125% to about 130%, such as about 130% to about 135%, such as about 135% to about 140%, such as about 140% to about 145%, such as about 145% to about 150%, such as about 150% to about 155%, such as about 155% to about 160%, such as about 160% to about 165%, such as about 165% to about 170%, such as about 170% to about 175%, such as about 175% to about 180% such as about 180% to about 185%, such as about 185% to about 190%, such as about 190% to about 195%, such as about 195% to about 200%, such as about 200% to about 205%, such as about 205% to about 210%, such as about 210% to about 215%, such as about 215% to about 220%, such as about 220% to about 225%, such as about 225% to about 230%, such as about 230% to about 235%, such as about 235% to about 240%, such as about 240% to about 245%, such as about 245% to about 250%, such as about 250% to about 255%, such as about 255% to about 260% such as about 260% to about 265%, such as about 265% to about 270%, such as about 270% to about 275%, such as about 275% to about 280%, such as about 280% to about 285%, such as about 285% to about 290%, such as about 290% to about 295%, such as about 295% to about 300%, such as about 300% to about 305%, such as about 305% to about 310%, such as about 310% to about 315%, such as about 315% to about 320%, such as about 320% to about 325%, such as about 325% to about 330%, such as about 330% to about 335%, such as about 335% to about 340%, such as about 340% to about 345%, such as about 345% to about 350%, such as about 350% to about 355%, such as about 355% to about 360%, such as about 360% to about 365%, such as about 365% to about 370%, such as about 370% to about 375%, such as about 375% to about 380%, such as about 380% to about 385%, such as about 385% to about 390%, such as about 390% to about 395%, such as about 395% to about 400%, such as about 400% to about 405%, such as about 405% to about 410%, such as about 410% to about 415%, such as about 415% to about 420%, such as about 420% to about 425%, such as about 425% to about 430%, such as about 430% to about 435%, such as about 435% to about 440%, such as about 440% to about 445%, such as about 445% to about 450% compared to said corresponding wild type MHC class I molecule.

The MHC molecules of humans are designated human leukocyte antigens (HLA). In humans, there are three major different genetic loci that encode MHC class I molecules: HLA-A, HLA-B, HLA-C. HLA-A*01, HLA-A*02, and HLA-A*11 are examples of MHC class I alleles that can be expressed from these loci. Non-classical human MHC class I molecules, such as HLA-E (homolog of mouse Qa-1b), CD1 and MR1 molecules are also encompassed by the present disclosure.

In some embodiments of the present disclosure, the MHC class I molecule is selected from the group consisting of HLA-A, HLA-B, HLA-C, HLA-E, HLA-F, HLA-G, MR1 and CD1.

In some embodiments of the present disclosure, the MHC class I molecule is an HLA-A molecule. In some embodiments of the present disclosure, the MHC class I molecule is an HLA-B molecule. In some embodiments of the present disclosure, the MHC class I molecule is an HLA-C molecule. In some embodiments of the present disclosure, the MHC class I molecule is an HLA-E molecule. In some embodiments of the present disclosure, the MHC class I molecule is an HLA-F molecule. In some embodiments of the present disclosure, the MHC class I molecule is an HLA-G molecule. In some embodiments of the present disclosure, the MHC class I molecule is an MR1 molecule.

In some embodiments of the present disclosure, the MHC class I molecule is a CD1 molecule.

In some embodiments of the present disclosure, the CD1 molecule is selected from the group consisting of CD1a, CD1b, CD1c and CD1d. Thus, in some embodiments of the present disclosure, the MHC class I molecule is a CD1a molecule. Thus, in some embodiments of the present disclosure, the MHC class I molecule is a CD1b molecule.

Thus, in some embodiments of the present disclosure, the MHC class I molecule is a CD1c molecule. Thus, in some embodiments of the present disclosure, the MHC class I molecule is a CD1d molecule.

In some embodiments of the present disclosure, the MHC class I molecule is an MHC class Ib molecule, such as an MHC class Ib molecule selected from the group consisting of MR1, HLA-E, HLA-F, HLA-G, CD1a, CD1b, CD1c and CD1d.

In specific embodiments, the MHC class I molecule of the present disclosure is an HLA-E or MR1 molecule.

In even more specific embodiments, the MHC class I molecule of the present disclosure is an HLA-E molecule.

The MHC class I molecules according to the present disclosure are in one embodiment of human origin and/or animal origin. In one embodiment, the MHC class I molecules according to the present disclosure is of mammal origin (e.g., macaque origin, rodent origin, such as mouse or rat origin). In another embodiment the MHC class I molecules according to the present disclosure is of non-mammalian vertebrate origin (such as from fish, bird, insect, amphibian, and/or reptile origin). In some embodiments, the MHC class I molecule according to the present disclosure is selected from the group consisting of human, non-human primates, Gorilla gorilla, Pan troglodytes, Macacca mulatta, Orangutang, Rodents, Mus musculus, Rattus norvegicus and Lagomorpha, such as rabbits and hares.

Thus, in some embodiments of the present disclosure, the MHC class I molecule is from a gorilla. In some embodiments of the present disclosure, the MHC class I molecule is from a chimpanzee. In some embodiments of the present disclosure, the MHC class I molecule is from a rhesus macaque. In some embodiments of the present disclosure, the MHC class I molecule is from a rodent. In some embodiments of the present disclosure, the MHC class I molecule is from a mouse. In some embodiments of the present disclosure, the MHC class I molecule is from a rat.

In some embodiments the MHC class I molecule of the present disclosure is a human MHC class I molecule.

### Monomers and Multimers

The present disclosure relates to MHC class I molecules in the form of monomers (MHC class I monomers) as disclosed herein above, as well as MHC class I multimers comprising a plurality of the MHC class I molecules as disclosed herein. MHC class I multimers and MHC class I/peptide multimers are described in detail in WO02072631, WO2008116468, WO2009003492 and WO2020127222, which are incorporated herein by reference.

The term “MHC class I monomer” refers to an MHC class I molecule according to the present disclosure, and the terms “MHC class I monomer” and “MHC class I molecule” are therefore used interchangeably herein. The term “MHC class I multimer” refers to a complex comprising multiple MHC class I monomers according to the present disclosure associated by covalent and/or noncovalent bonds. The MHC class I monomers comprised in the MHC class I multimer can be substantially identical MHC class I monomers, or the MHC class I monomers may be different. In some embodiments of the present disclosure, the MHC class I multimer is a dimer, a trimer, a tetramer, a pentamer, a hexamer, a heptamer or an octamer or any higher valency multimer, e.g., comprising 4, 5, 6, 7, 8, 9, 10, 11, 12, 13, 14, 15, 16, 17, 18, 19, 20, 21, 22, 23, 24 or more than 24 monomers according to the present disclosure.

In some embodiments, the MHC class I monomer and the MHC class I multimer according to the present disclosure comprise a peptide in the binding groove of the MHC class I molecule(s), referred to as MHC class I/peptide monomers and MHC class I/peptide multimers, respectively. Accordingly, as used herein, the term “MHC class I/peptide multimer” refers to a multimer such as a stable multimeric complex composed of or comprising MHC class I monomers according to the present disclosure each loaded with a peptide (i.e., MHC class I/peptide monomers). For example, an MHC class I/peptide multimer include, but are not limited to, an MHC class I/peptide dimer, trimer, tetramer, pentamer, hexamer, heptamer or octamer or any higher valency multimer, e.g., comprising 4, 5, 6, 7, 8, 9, 10, 11, 12, 13, 14, 15, 16, 17, 18, 19, 20, 21, 22, 23, 24 or more than 24 MHC class I/peptide monomers.

In some aspects the present disclosure provides an MHC class I multimer or an MHC class I/peptide multimer comprising at least two MHC class I monomers according to the present disclosure.

In some embodiments of the present disclosure, the MHC class I multimer or MHC class I/peptide multimer comprises at least two identical MHC class I monomers according to the present disclosure. In some embodiments of the present disclosure, the MHC class I multimer or the MHC class I/peptide multimer comprises at least 3, such as at least 4, 5, 6, 7, 8, 9, 10, 11, 12, 13, 14, 15, 16, 17, 18, 19 or20 identical MHC class I monomers.

In some embodiments of the present disclosure, the MHC class I multimer or MHC class I/peptide multimer comprises at least two different MHC class I monomers according to the present disclosure. In some embodiments of the present disclosure, the MHC class I multimer or the MHC class I/peptide multimer comprises at least 3, such as at least 4, 5, 6, 7, 8, 9, 10, 11, 12, 13, 14, 15, 16, 17, 18, 19 or 20 different MHC class I monomers.

The MHC class I/peptide monomers in a multimer can be substantially identical MHC class I/peptide monomers, or the MHC class I/peptide monomers may be different.

Each MHC class I monomer of the MHC class I multimer can be associated with one or more multimerization domains, such as a multimerization domain selected from the group consisting of IgG, streptavidin, avidin, streptactin, micelles, cells, polymers, dextran, polysaccharides, beads and other types of solid support, and small organic molecules carrying reactive groups or carrying chemical motifs that can bind MHC class I monomers. Likewise, each MHC class I/peptide monomer of the MHC class I/peptide multimer can be associated with one or more multimerization domains such as a multimerization domain selected from the group consisting of IgG, streptavidin, avidin, streptactin, micelles, cells, polymers, dextran, polysaccharides, beads and other types of solid support, and small organic molecules carrying reactive groups or carrying chemical motifs that can bind MHC class I monomers.

The term “multimerization domain” as used herein can be any type of molecule that is directly or indirectly associated with one or more MHC class I monomers and/or MHC class I/peptide monomers. A multimerization domain is a molecule, a complex of molecules, or a solid support, to which one or more MHC class I and/or MHC class I/peptide monomers can be attached. A multimerization domain can consist of one or more carriers and/or one or more scaffolds and may also contain one or more linkers connecting carrier to scaffold, carrier to carrier, and/or scaffold to scaffold. The multimerization domain may also contain one or more linkers that can be used for attachment of MHC class I monomers and/or MHC class I/peptide monomers and/or other molecules to the multimerization domain. In this disclosure, a multimerization domain will in one embodiment refer to a functionalized polymer (e.g., dextran) that is capable of reacting with MHC class I monomers and/or MHC class I/peptide monomers, thus covalently attaching the MHC class I monomer and/or MHC class I/peptide monomer to the multimerization domain, or that is capable of reacting with scaffold molecules (e.g., streptavidin), thus covalently attaching streptavidin to the multimerization domain; the streptavidin then may bind the MHC class I monomers and/or MHC class I/peptide monomers. Multimerization domains include IgG, streptavidin, avidin, streptactin, micelles, cells, polymers, dextran, polysaccharides, beads and other types of solid support, and small organic molecules carrying reactive groups or carrying chemical motifs that can bind MHC class I monomers and/or MHC class I/peptide monomers and other molecules, such as identified in detail herein elsewhere.

Non-limiting examples of suitable multimerization domain(s) are polysaccharides including dextran molecules, carboxy methyl dextran, dextran polyaldehyde, carboxymethyl dextran lactone, and cyclodextrins, pullulans, schizophyllan, scleroglucan, xanthan, gellan, O-ethylamino guaran, chitins and chitosans inducing 6-O-carboxymethyl chitin and N-carboxymethyl chitosan, derivatised cellolosics including carboxymethyl cellulose, carboxymethyl hydroxyethyl cellulose, hydroxy- ethyl cellulose, 6-amino-6-deoxy cellulose and O-ethyl- amine cellulose, hydroxylated starch, hydroxypropyl starch, hydroxyethyl starch, carrageenans, alginates, and agarose, synthetic polysaccharides including ficoll and carboxy-methylated ficoll, vinyl polymers including poly (acrylic acid), poly (acryl amides), poly (acrylic esters), poly (2-hydroxy ethyl methacrylate), poly (methyl methacrylate), poly (maleic acid), poly (maleic anhydride), poly (acrylamide), poly (ethyl-co- vinyl acetate), poly (methacrylic acid), poly (vinyl- alcohol), poly (vinyl alcohol-co-vinyl chloroacetate), aminated poly (vinyl alcohol), and co block polymers thereof, poly ethylene glycol (PEG) or polypropylene glycol or poly (ethylene oxide-co-propylene oxides) comprising polymer backbones including linear, comb-shaped or StarBurst dendrimers, poly amino acids including polylysines, polyglutamic acid, polyurethanes, poly (ethylene imines), pluriol, proteins including peptides, polypeptides, antigen binding peptides, albumins, immunoglobulins, coiled-coil helixes e.g. Fos-Jun or Fos-Jun like or coiled-coiled dimers/trimers/tetramers/pentamers, streptavidin, avidin, streptactin, T-cell receptors, other protein receptors and virus-like proteins (VLP), and polynucleotides, DNA, RNA, PNA, LNA, oligonucleotides and oligonucleotide dendrimer constructs and small organic molecules including but not limited to steroids, peptides, linear or cyclic structures, aromatic structures, aliphatic structures.

The term “Dextran” as used herein is a complex, branched polysaccharide made of glucose molecules joined into chains of varying lengths. The straight chain consists of α1->6 glycosidic linkages between glucose molecules, while branches begin from α1->3 linkages (and in some cases, α1->2 and α1->4 linkages as well).

In one embodiment of the present disclosure, the MHC class I multimer comprises at least 2 MHC class I monomers, such as at least 3 MHC class I monomers, such as at least 4 MHC class I monomers, such as at least 5 MHC class I monomers, such as at least 6 MHC class I monomers, such as at least 7 MHC class I monomers, such as at least 8 MHC class I monomers, such as at least 9 MHC class I monomers, such as at least 10 MHC class I monomers, such as at least 11 MHC class I monomers, such as at least 12 MHC class I monomers, such as at least 13 MHC class I monomers, such as at least 14 MHC class I monomers, such as at least 15 MHC class I monomers, such as at least 16 MHC class I monomers, such as at least 17 MHC class I monomers, such as at least 18 MHC class I monomers, such as at least 19 MHC class I monomers, or such as at least 20 MHC class I monomers.

In another embodiment of the present disclosure, the MHC class I multimer comprises 2 to 50 MHC class I monomers, such as 10 to 20 MHC class I monomers, such as 2 to 4 MHC class I monomers, such as 4 to 5 MHC class I monomers, such as 5 to 6 MHC class I monomers, such as 6 to 8 MHC class I monomers, such as 8 to 10 MHC class I monomers, such as 10 to 12 MHC class I monomers, such as 12 to 14 MHC class I monomers, such as 14 to 16 MHC class I monomers, such as 16 to 18 MHC class I monomers, such as 18 to 20 MHC class I monomers, such as 20 to 25 MHC class I monomers, such as 25 to 30 MHC class I monomers, such as 30 to 40 MHC class I monomers, such as 40 to 50 MHC class I monomers of the present disclosure, or any combination of these intervals.

In some embodiments of the present disclosure, the MHC class I multimer comprises no more than 30 MHC class I monomers, such as no more than 25 MHC class I monomers, such as no more than 20 MHC class I monomers, such as no more than 15 MHC class I monomers, or no more than 10 MHC class I monomers.

In one embodiment of the present disclosure, the MHC class I/peptide multimer comprises at least 2 MHC class I/peptide monomers, such as at least 3 MHC class I/peptide monomers such as at least 4 MHC class I/peptide monomers, such as at least 5 MHC class I/peptide monomers, such as at least 6 MHC class I/peptide monomers, such as at least 7 MHC class I/peptide monomers, such as at least 8 MHC class I/peptide monomers, such as at least 9 MHC class I/peptide monomers, such as at least 10 MHC class I/peptide monomers, such as at least 11 MHC class I/peptide monomers, such as at least 12 MHC class I/peptide monomers, such as at least 13 MHC class I/peptide monomers, such as at least 14 MHC class I/peptide monomers, such as at least 15 MHC class I/peptide monomers, such as at least 16 MHC class I/peptide monomers, such as at least 17 MHC class I/peptide monomers, such as at least 18 MHC class I/peptide monomers, such as at least 19 MHC class I/peptide monomers, or such as at least 20 MHC class I/peptide monomers of the present disclosure.

In another embodiment the MHC class I/peptide multimer of the present disclosure comprises 2 to 50 MHC class I/peptide monomers, such as 10 to 20 MHC class I/peptide monomers, such as 4 to 6 MHC class I/peptide monomers, such as 6 to 8 MHC class I/peptide monomers, such as 8 to 10 MHC class I/peptide monomers, such as 10 to 12 MHC class I/peptide monomers, such as 12 to 14 MHC class I/peptide monomers, such as 14 to 16 MHC class I/peptide monomers, such as 16 to 18 MHC class I/peptide monomers, such as 18 to 20 MHC class I/peptide monomers, such as 20 to 25 MHC class I/peptide monomers, such as 25 to 30 MHC class I/peptide monomers, such as 30 to 40 MHC class I/peptide monomers, such as 40 to 50 MHC class I/peptide monomers of the present disclosure, or any combination of these intervals.

In some embodiments of the present disclosure, the MHC class I/peptide multimer comprises no more than 30 MHC class I/peptide monomers, such as no more than 25 MHC class I/peptide monomers, such as no more than 20 MHC class I/peptide monomers, such as no more than 15 MHC class I/peptide monomers, or no more than 10 MHC class I/peptide monomers.

In a specific embodiment of the present disclosure, the MHC class I multimer and/or the MHC class I/peptide multimer comprises at least 2, 3, 4, 5, 6, 7, 8, 9, 10, 11, 12, 13, 14, 15, 16, 17, 18, 19, 20, 21, 22, 23, 24 or 25 MHC class I monomers and/or MHC class I/peptide monomers of the present disclosure, respectively.

In a specific embodiment of the present disclosure, the MHC class I multimer and/or the MHC class I/peptide multimer has 2, 3, 4, 5, 6, 7, 8, 9, 10, 11, 12, 13, 14, 15, 16, 17, 18, 19, 20, 21, 22, 23, 24 or 25 MHC class I monomers and/or MHC class I/peptide monomers of the present disclosure, respectively.

In one embodiment, the MHC class I multimer comprises identical MHC class I monomers or all MHC class I monomers of the MHC class I multimer can be identical. In another embodiment the MHC class I multimer comprises different MHC class I monomers or all MHC class I monomers of the MHC class I multimer are different. In another embodiment the MHC class I/peptide multimer comprises identical MHC class I/peptide monomers or all MHC class I/peptide monomers of the MHC class I/peptide multimer are identical. In another embodiment the MHC class I/peptide multimer comprises different MHC class I/peptide monomers or all MHC class I/peptide monomers of the MHC class I/peptide multimer are different.

In one embodiment some of the MHC class I/peptide monomers or all of the MHC class I/peptide monomers comprised in an MHC class I/peptide multimer comprise identical peptides. In another embodiment some of the MHC class I/peptide monomers or all of the MHC class I/peptide monomers comprised in an MHC class I/peptide multimer comprise different peptides. In one embodiment the MHC class I/peptide multimer of the present disclosure comprises at least 2, such as at least 3, 4, 5, 6, 7, 8, 9, 10, 11, 12, 13, 14, 15, 16, 17, 18, 19 or 20 MHC class I/peptide monomers each of which comprise different peptides.

In one embodiment, the MHC class I multimer and/or MHC class I/peptide multimer of the present disclosure comprises one or more labels. In one embodiment the MHC class I multimer and/or MHC class I/peptide multimer of the present disclosure comprises at least two labels. These labels can all be different or identical, or some the labels can be identical and some different. In some embodiments of the present disclosure, all labels are different or at least two of the labels are different. In some embodiments of the present disclosure, all labels are identical or at least two of the labels are identical.

In one embodiment of the present disclosure, the one or more labels comprise at least one fluorescent label and/or at least one oligonucleotide label.

In a specific embodiment of the present disclosure, the at least one oligonucleotide label comprises one or more of: a 5′ first primer region (forward), a barcode region, 3′ second primer region (reverse), random nucleotide region, connector molecule, stability-increasing components, short nucleotide linkers in between any of the above-mentioned components, adaptors for sequencing and annealing region.

In one embodiment the one or more labels are directly attached to the MHC class I multimer and/or MHC class I/peptide multimer. In one embodiment the one or more labels are indirectly attached to the MHC class I multimer and/or MHC class I/peptide multimers, such as via one or more marker molecules carrying one or more labels.

The one or more labels may be used for combinatorial use of labelling. The one or more labels may result in positive selection of said MHC class I/peptide multimer or alternatively in negative selection of said MHC class I/peptide multimer. The one or more labels may comprise one or more covalently attached labels and/or one or more non-covalently attached labels. The one or more labels may be covalently attached to “polypeptide a” of the MHC class I monomer and/or MHC class I/peptide monomer, covalently attached to “polypeptide b” of the MHC class I monomer and/or MHC class I/peptide monomer, covalently attached to the peptide and/or covalently attached to the one or more multimerization domains. Alternatively, the one or more labels may be non-covalently attached to “polypeptide a” of the MHC class I monomer and/or MHC class I/peptide monomer, non-covalently attached to “polypeptide b” of the MHC class I monomer and/or MHC class I/peptide monomer, non-covalently attached to the peptide and/or non-covalently attached to the one or more multimerization domains. In another embodiment of the present disclosure, the one or more labels may be covalently and/or non-covalently attached to the multimerization domain via a molecule, wherein the molecule e.g., may be selected from the group consisting of an antibody, an aptamer, a protein, a sugar residue, and a nucleotide such as DNA. In a specific embodiment the one or more labels are attached to the MHC class I multimer and/or MHC class I/peptide multimer via a streptavidin-biotin linkage.

In embodiments of the present disclosure, wherein multimers comprising at least two MHC class I monomers and/or MHC class I/peptide monomers comprise both at least a fluorescent label and at least an oligonucleotide label, each fluorescent label may be identical.

In embodiments of the present disclosure, wherein multimers comprising at least two MHC class I monomers and/or MHC class I/peptide monomers comprise both at least a fluorescent label and at least an oligonucleotide label, each fluorescent label may be different.

In embodiments of the present disclosure, wherein multimers comprising at least two MHC class I monomers and/or MHC class I/peptide monomers comprise both at least a fluorescent label and at least an oligonucleotide label, each oligonucleotide label may be identical.

In embodiments of the present disclosure, wherein multimers comprising at least two MHC class I monomers and/or MHC class I/peptide monomers comprise both at least a fluorescent label and at least an oligonucleotide label, each oligonucleotide label may be different.

In embodiments of the present disclosure, wherein multimers comprising at least two MHC class I monomers and/or MHC class I/peptide monomers comprise both at least a fluorescent label and at least an oligonucleotide label, each fluorescent label and each oligonucleotide label may be identical.

In embodiments of the present disclosure, wherein multimers comprising at least two MHC class I monomers and/or MHC class I/peptide monomers comprise both at least a fluorescent label and at least an oligonucleotide label, each fluorescent label and each oligonucleotide label may be different.

In embodiments of the present disclosure, wherein multimers comprising at least two MHC class I monomers and/or MHC class I/peptide monomers comprise both at least a fluorescent label and at least an oligonucleotide label, each fluorescent label may be identical and each oligonucleotide label may be different.

In embodiments of the present disclosure, wherein multimers comprising at least two MHC class I monomers and/or MHC class I/peptide monomers comprise both at least a fluorescent label and at least an oligonucleotide label, each fluorescent label may be different and each oligonucleotide label may be identical.

The term “Label” is used interchangeable with labeling molecule. Label as described herein is an identifiable substance that is detectable in an assay and that can be attached to a molecule creating a labeled molecule. The behavior of the labeled molecule can then be studied. Labels may be organic or inorganic molecules or particles. Examples of labels include, but are not limited to, polymers, nucleic acids, DNA, RNA, oligonucleotides, peptides, fluorescent labels, phosphorescent labels, enzyme labels, chemiluminescent labels, bioluminescent labels, haptens, antibodies, dyes, nanoparticle labels, elements, metal particles, heavy metal labels, isotope labels, radioisotopes, stable isotopes, chains of isotopes and single atoms, or combination thereof. The labelling compound may suitably be selected from fluorescent labels such as 5-(and 6)-carboxyfluorescein, 5- or 6-carboxyfluorescein, 6-(fluorescein)-5-(and 6)-carboxamido hexanoic acid, fluorescein isothiocyanate (FITC), rhodamine, tetramethylrhodamine, and dyes such as Cy2, Cy3, and Cy5, optionally substituted coumarin including AMCA, PerCP, phycobiliproteins including R-phycoerythrin (RPE) and allophycoerythrin (APC), Texas Red, Princeston Red, Green fluorescent protein (GFP) and analogues thereof, and conjugates of R-phycoerythrin or allophycoerythrin and e.g. Cy5 or Texas Red, and inorganic fluorescent labels based on semiconductor nanocrystals (like quantum dot and Qdot™ nanocrystals), and time-resolved fluorescent labels based on lanthanides like Eu³⁺ and Sm³⁺.

In one embodiment an MHC class I monomer or MHC class I multimer according to the present disclosure comprises at least one nucleic acid label, such as a nucleotide label, for example an oligonucleotide label. Such nucleic acids labels are disclosed in WO 2015/188839 and WO 2015/185067 (which are hereby incorporated by reference).

In a particular embodiment the label is an oligonucleotide, such as a nucleic acid molecule comprising or consisting of DNA, RNA, and/or artificial nucleotides such as PLA or LNA. In one embodiment the nucleic acid label comprises one or more of the following components: a 5′ first primer region (forward), a barcode region, 3′ second primer region (reverse), random nucleotide region, connector molecule, stability-increasing components, short nucleotide linkers in between any of the above-mentioned components, adaptors for sequencing and annealing region.

Preferably the nucleic acid label comprises at least a barcode region surrounded by primer regions, where the barcode region comprises a sequence of consecutive nucleic acids. In one embodiment the nucleic acid label comprises or consists of DNA, RNA, artificial nucleic acids and/or Xeno nucleic acid (XNA).

In one embodiment at least two different labels are attached to an MHC class I monomer, an MHC class I/peptide monomer, an MHC class I multimer or an MHC class I/peptide multimer according to the present disclosure, such as one or more fluorescent labels and a nucleic acid label.

In some embodiments, the MHC class I multimer and/or MHC class I/peptide multimer according to the preset disclosure comprises one or more fluorescent labels. In some embodiments, the MHC class I multimer and/or MHC class I/peptide multimer according to the preset disclosure comprises one or more fluorescent labels selected from the group of fluorescein isothiocyanate, rhodamine, phycoerythrin, phycocyanin, allophycocyanin, o-phthaldehyde, fluorescamine, 2-4′-maleimidylanilino)naphthalene-6-sulfonic acid sodium salt, 5-((((2-iodoacetyl)amino)ethyl)amino), naphthalene-1-sulfonic acid, Pyrene-1-butanoic acid, AlexaFluor 350 (7-amino-6-sulfonic acid-4-methyl coumarin-3-acetic acid, AMCA (7-amino-4-methyl coumarin-3-acetic acid), 7-hydroxy-4-methyl coumarin-3-acetic acid, Marina Blue (6,8-difluoro-7-hydroxy-4-methyl coumarin-3-acetic acid), 7-dimethylamino-coumarin-4-acetic acid, Fluorescamin-N-butyl amine adduct, 7-hydroxy-coumarine-3-carboxylic acid, CascadeBlue (pyrene-trisulphonic acid acetyl azide), Cascade Yellow, Pacific Blue (6,8 difluoro-7-hydroxy coumarin-3-carboxylic acid), 7-diethylamino-coumarin-3-carboxylic acid, N-(((4-azidobenzoyl)amino)ethyl)-4-amino-3,6-disulfo-1,8-naphthalimide, dipotassium salt), Alexa Fluor 430, 3-perylenedodecanoic acid, 8-hydroxypyrene-1,3,6-trisulfonic acid trisodium salt, 12-(N-(7-nitrobenz-2-oxa-1,3-diazol-4-yl)amino)dodecanoic acid, N,N′-dimethyl-N- (iodoacetyl)-N′-(7-nitrobenz-2-oxa-1,3-diazol-4-yl)ethylenediamine, Oregon Green 488 (difluoro carboxy fluorescein), 5-iodoacetamidofluorescein, propidium iodide-DNA adduct, Carboxy fluorescein, fluor dyes, Pacific Blue™, Pacific Orange™, Cascade Yellow™, AlexaFluor®(AF), AF350, AF405, AF430, AF488,AF500, AF514, AF532, AF546, AF555, AF568, AF594, AF610, AF633, AF635, AF647, AF680, AF700, AF710, AF750, AF800, Quantum Dot based dyes, QDot® Nanocrystals (Invitrogen, MolecularProbs), Qdot®525, Qdot®565, Qdot®585, Qdot*605, Qdot*655, Qdot*705, Qdot*800, DyLight™ Dyes (Pierce) (DL); DL549, DL649, DL680, DL800, Fluorescein (Flu) or any derivate of that, such as FITC, Cy-Dyes, Cy2, Cy3, Cy3.5, Cy5, Cy5.5, Cy7, 7-AAD, TO-Pro-3, fluorescent Proteins, R-Phycoerythrin (RPE), Phycobili Proteins, Allophycocyani (APC), PerCp, B-Phycoerythrin, C-Phycocyanin, APC, fluorescent proteins, Green fluorescent proteins; GFP and GFP derivated mutant proteins; BFP,CFP, YFP, DsRed, DSred-2, T1, Dimer2, mRFP1, MBanana, mOrange, dTomato, tdTomato, mTangerine, mStrawberry, mCherry, Tandem dyes, RPE-Cy5, RPE-Cy5.5, RPE-Cy7, RPE-AlexaFluor® tandem conjugates; RPE-Alexa610, RPE-TxRed, Tandem dyes with APC, APC-Aleca600, APC-Alexa610, APC-Alexa750, APC-Cy5, APC-Cy5.5, multi fluorochrome assemblies, FRET-based dyes (Fluorescence resonance energy transfer), ionophors; ion chelating fluorescent props, props that change wavelength when binding a specific ion, such as Calcium, props that change intensity when binding to a specific ion, such as Calcium, Calcium dyes, Indo-1-Ca²⁺ Indo-2-Ca²⁺.

The one or more labels are in specific embodiments selected from the group consisting of APC, APC-Cy7, ABC-H7, APC-R700, Alexa Flours™ 488, Alexa Flours™555, Alexa Flours™647, Alexa Flours™700, AmCyan, BB151, BB700, BUV395, BUV496, BUV563, BUV615, BUV661, BUV737, BUV805, BV421, BV480, BV510, BV605, BV711, BV750, BV786, FITC, PE, PE-CF594, PE-Cy5, PE-CY5.5, PE-cy7, Pasific Blue, PERCP, pPerCp-Cy5.5, PE, R718, RY586, V450 and V500 (wherein in BV means Brilliant violet, wherein BUV means Brilliant ultra violet and PE means R-Phycoerythrin). In another embodiment the one or more labels can be selected from the group consisting of cFluor®B515, cFluor®B532, cFluor®B548, cFluor®B675, cFluor®B690, cFluor®BY575, cFluor®BY610, cFluor®BY667, cFluor®BY710, cFluor®BY750, cFluor®BY781, cFluor®B250, cFluor®R659, cFluor®R668, cFluor®R685, cFluor®R720, cFluor®R780, cFluor®R840, cFluor®v420, cFluor®v547, cFluor®v450, cFluor®v610 and cFluor*YG610.

In one embodiment, the MHC class I multimer and/or MHC class I/peptide multimer of the present disclosure comprises one or more chemiluminescent labels, such as one or more labels selected from the group consisting of luminol, isoluminol, theromatic acridinium ester, imidazole, acridinium salt and oxalate ester.

In one embodiment the MHC class I multimer and/or MHC class I/peptide multimer of the present disclosure comprises one or more bioluminescent labels, such as one or more labels selected from the group consisting of luciferin, luciferase and aequorin.

In one embodiment the MHC class I multimer and/or MHC class I/peptide multimer of the present disclosure comprises one or more enzyme labels, such as one or more enzyme labels selected from the group peroxidases, malate dehydrogenase, staphylococcal nuclease, delta-5-steroid isomerase, yeast alcohol dehydrogenase, alpha-glycerophosphate, dehydrogenase, triose phosphate isomerase, horseradish peroxidase, alkaline phosphatase, asparaginase, glucose oxidase, beta-galactosidase, ribonuclease, urease, catalase, glucose-6-phosphate dehydrogenase, glucoamylase and acetylcholinesterase.

In one embodiment the MHC class I multimer and/or MHC class I/peptide multimer of the present disclosure comprises one or more chromophore labels.

In one embodiment the MHC class I multimer and/or MHC class I/peptide multimer of the present disclosure comprises one or more metal labels.

In one embodiment the MHC class I multimer and/or MHC class I/peptide multimer of the present disclosure comprises one or more radioactive labels such as one or more labels selected from the group consisting of a radionuclide, an isotope, a label comprising a rays, a label comprising p rays or a label comprising y rays. Any of the above embodiments regarding labels can be combined in any order.

### Composition

The present disclosure further relates to a composition comprising one or more MHC class I molecules, one or more MHC class I monomers, one or more MHC class I/peptide monomers, one or more MHC class I multimers and/or one or more MHC class I/peptide multimers according to the present disclosure.

In some embodiments of the present disclosure, the composition comprises at least 1, such as at least 2, 3, 4, 5, 6, 7, 8, 9, 10, 20, 30, 40, 50, 100 or 1000 MHC class I molecules, MHC class I monomers, MHC class I/peptide monomers, MHC class I multimers and/or MHC class I/peptide multimers according to the present disclosure.

In some embodiments of the present disclosure, the composition comprises at least 2, 3, 4, 5, 6, 7, 8, 9, 10, 20, 30, 40, 50, 100 or 1000 identical MHC class I molecules, MHC class I monomers, MHC class I/peptide monomers, MHC class I multimers and/or MHC class I/peptide multimers according to the present disclosure.

In some embodiments of the present disclosure, the composition comprises at least 2, 3, 4, 5, 6, 7, 8, 9, 10, 20, 30, 40, 50, 100 or 1000 different MHC class I molecules, MHC class I monomers, MHC class I/peptide monomers, MHC class I multimers and/or MHC class I/peptide multimers according to the present disclosure.

In some embodiments of the present disclosure, the composition further comprises one or more pH-stabilizing (buffer) components, such as one or more pH-stabilizing (buffer) components selected from the group consisting of Tris, MES, MOPS, phosphate, carbonate, Bis-tris and HEPES.

In some embodiments of the present disclosure, the composition further comprises one or more salts, such as one or more salts selected from the group consisting of NaCl, CaCl₂, and L-arginine.

In some embodiments of the present disclosure, the composition further comprises one or more stabilizers, such as one or more stabilizers selected from the group consisting of glycerol, PEG and BSA.

In specific embodiments of the present disclosure, the composition comprises one or more pH-stabilizing (buffer) components, one or more salts and one or more stabilizers.

In some embodiments of the present disclosure, the composition is a pharmaceutical composition comprising a pharmaceutically acceptable diluent, carrier and/or excipient.

### Methods/Medical Uses

The present disclosure provides MHC class I molecules, such as MHC class I monomers, MHC class I/peptide monomers, MHC class I multimers and/or MHC class I/peptide multimers.

Thus, in some aspects the present disclosure provides a method of stabilizing and/or increasing the refolding yield of an MHC class I molecule,
wherein said MHC class I comprises or consists of at least amino acids 1-51 and 91-137 of the amino acid sequence as set forth in SEQ ID NO: 1 or 2, or a variant thereof having at least 85% sequence homology or identity thereto, such as at least 90% sequence homology or identity thereto, such as at least 95% sequence homology or identity thereto, or such as at least 98% sequence homology or identity to SEQ ID NO: 1 or 2,
said method comprising the step of mutating the amino acids corresponding to positions
   i) 4 and 102 of SEQ ID NO: 1 or 2, and/or
   ii) 12 and 94 of SEQ ID NO: 1 or 2, and/or
   iii) 23 and 37 of SEQ ID NO: 1 or 2, and/or
   iv) 115 and 125 of SEQ ID NO: 1 or 2
      to cysteines and linking each of the cysteines of i) with a disulphide bridge, each of the cysteines of ii) with a disulphide bridge, each of the cysteines of iii) with a disulphide bridge and/or each of the cysteines of iv) with a disulphide bridge.

In some embodiments of the present disclosure, said MHC class I molecule is an HLA-E molecule. In some embodiments of the present disclosure, said MHC class I molecule is an HLA-F molecule. In some embodiments of the present disclosure, said MHC class I molecule is an HLA-G molecule.

The MHC class I monomers, MHC class I/peptide monomers, MHC class I multimers and/or MHC class I/peptide multimers according to the present disclosure are particularly suitable for isolating, detecting, and/or monitoring immune cells, such as T cells, CD8+ T cells, MAIT cells and/or NK cells and/or immune responses. The MHC class I monomers, MHC class I/peptide monomers, MHC class I multimers and/or MHC class I/peptide multimers according to the present disclosure may also be used for diagnosis of diseases.

In some aspects, the present disclosure provides a method for isolation of one or more immune cells, such as antigen-specific T cells, CD8+ T cells, MAIT cells and/or NK cells, said method comprising the steps of:
a) providing a sample comprising a population of immune cells, such as T cells;
b) providing one or more MHC class I monomers, MHC class I/peptide monomers, MHC class I multimers and/or MHC class I/peptide multimers according to the present disclosure, or a composition comprising one or more of said MHC class I monomers, MHC class I/peptide monomers, MHC class I multimers and/or MHC class I/peptide multimers;
c) contacting said MHC class I monomers, MHC class I/peptide monomers, MHC class I multimers and/or MHC class I/peptide multimers, or said composition, with said sample; and
d) isolating said immune cells, such as T cells specific for said MHC class I monomers, MHC class I/peptide monomers, MHC class I multimers and/or MHC class I/peptide multimers, or composition.

In some aspects, the present disclosure provides a method for detecting an immune cell response, such as an antigen-specific T cell response, the method comprising the steps of:
a) providing a sample comprising a population of immune cells, such as T cells;
b) providing one or more MHC class I monomers, MHC class I/peptide monomers, MHC class I multimers and/or MHC class I/peptide multimers according to the present disclosure, or a composition comprising one or more of said MHC class I monomers, MHC class I/peptide monomers, MHC class I multimers and/or MHC class I/peptide multimers:
c) contacting said MHC class I monomers, MHC class I/peptide monomers, MHC class I multimers and/or MHC class I/peptide multimers, or said composition, with said sample; and
d) measuring the presence, frequency, number, activity and/or state of immune cells, such as T cells specific for said MHC class I monomers, MHC class I/peptide monomers, MHC class I multimers and/or MHC class I/peptide multimers, or composition,
   thereby detecting said immune cell response, such as said antigen-specific T cell response.

In some embodiments, the immune cell is an antigen-specific T cell, a CD8+ T cell, an MAIT cells and/or an NK cells.

In some aspects, the present disclosure provides a method for monitoring an immune response, the method comprising the steps of:
a) providing a sample comprising a population of immune cells, such as T cells;
b) providing one or more MHC class I monomers, MHC class I/peptide monomers, MHC class I multimers and/or MHC class I/peptide multimers according to the present disclosure, or a composition comprising one or more of said MHC class I monomers, MHC class I/peptide monomers, MHC class I multimers and/or MHC class I/peptide multimers; and
c) contacting said MHC class I monomers, MHC class I/peptide monomers, MHC class I multimers and/or MHC class I/peptide multimers, or said composition, with said sample; and
d) measuring the presence, frequency, number, activity and/or state of the immune cells, such as T cells, specific for said one or more MHC class I monomers, MHC class I/peptide monomers, MHC class I multimers and/or MHC class I/peptide multimers, thereby monitoring said immune response.

In some embodiments, the immune cell is an antigen-specific T cell, a CD8+ T cell, a MAIT cell and/or an NK cell.

In some aspects, the present disclosure provides a method for diagnosing a disease, the method comprising the steps of:
a) providing a sample comprising a population of immune cells, such as T cells;
b) providing one or more MHC class I monomers, MHC class I/peptide monomers, MHC class I multimers and/or MHC class I/peptide multimers according to the present disclosure, or a composition comprising one or more of said MHC class I monomers, MHC class I/peptide monomers, MHC class I multimers and/or MHC class I/peptide multimers;
c) contacting said MHC class I monomers, MHC class I/peptide monomers, MHC class I multimers and/or MHC class I/peptide multimers, or said composition, with said sample; and
d) measuring the presence, frequency, number, activity and/or state of immune cells, such as T cells, specific for said one or more MHC class I monomers, MHC class I/peptide monomers, MHC class I multimers and/or MHC class I/peptide multimers,
   thereby diagnosing said disease.

In some embodiments, the immune cell is an antigen-specific T cell, a CD8+ T cell, a MAIT cell and/or an NK cell.

The term “sample” refers to e.g., a liquid sample or a solid sample. A liquid sample include, but is not limited to, blood, lymph, cerebrospinal fluid, synovial fluid, sputum, lymph, semen, fluid cultures of cells, suspensions of solid tissue. Examples of a solid sample include, but is not limited to, solid tissue, tissue sections, organ, part of organ or tissue, human bodies or any part thereof, animal bodies of any part thereof and cells embedded in a solid matrix, e.g., paraffin. The sample can be derived from a human or an animal. The sample is preferably a biological sample.

In some embodiments of the present disclosure, the sample is blood. In some embodiments of the present disclosure, the sample is lymph. In some embodiments of the present disclosure, the sample is cerebrospinal fluid. In some embodiments of the present disclosure, the sample is synovial fluid. In some embodiments of the present disclosure, the sample is sputum. In some embodiments of the present disclosure, the sample is lymph. In some embodiments of the present disclosure, the sample is semen. In some embodiments of the present disclosure, the sample is fluid cultures of cells. In some embodiments of the present disclosure, the sample is suspensions of solid tissue, such as suspensions of tissue sections, organs, part of organs or tissues, human bodies or any part thereof, animal bodies of any part thereof, and cells embedded in a solid matrix, e.g., paraffin.

The MHC class I monomers, MHC class I/peptide monomers, MHC class I multimers and/or MHC class I/peptide multimers of the present disclosure can also be used as therapeutic agents.

In some aspects, the present disclosure thus provides an MHC class I monomer, an MHC class I/peptide monomer, an MHC class I multimer, or an MHC class I/peptide multimer of the present disclosure, or a combination thereof, for use as a medicament.

In some embodiments the MHC class I monomers, MHC class I/peptide monomers, MHC class I multimers and/or MHC class I/peptide multimers of the present disclosure can prevent NK cell cytotoxicity.

In a preferred embodiment the ability of certain MHC class I molecules, such as HLA-E, to prevent NK cell cytotoxicity is exploited in the context of tissue transplants or adoptive cell therapy. Eliminating expression of native MHC expression in a transplant can prevent cells or tissues from being rejected by the adaptive immune system of the recipient (e.g., by using β₂m knockout). To simultaneously prevent NK cell cytotoxicity, MHC class I molecules, such as HLA-E, can be reintroduced into the transplanted tissue, e.g., as a single-chain construct (typically β₂m-linker-HC; before or after transplantation to the recipient), optionally along with a source of HLA signal peptide fragments (either separately expressed or expressed as part of the introduced HLA-E protein). Using a stabilized form of the MHC class I molecule, e.g. a stabilized form of HLA-E, as provided by the present disclosure, allows for higher surface expression levels of MHC class I complexes, such as HLA-E complexes, which increases the ability to pacify NK cells and thus prevents tissue rejection.

In some aspects the present disclosure provides an MHC class I monomer, an MHC class I/peptide monomer, an MHC class I multimer, or an MHC class I/peptide multimer according to the present disclosure, or a combination thereof, for use in a method of limiting or preventing NK cell cytotoxicity during or following tissue transplantation of a transplant tissue into a recipient, such as a human being.

Accordingly, in one aspect of the present disclosure is provided a method for limiting or preventing NK cell cytotoxicity during or following tissue transplantation of a transplant tissue into a recipient, said method comprising the steps of:
a) eliminating expression of native MHC molecules in the transplant tissue prior to transplantation of said transplant tissue into the recipient;
b) transplanting said transplant tissue into the recipient, wherein the method further comprises the steps of:
c) introducing an MHC class I molecule according to the present disclosure into said transplant tissue prior to or after transplantation of said transplant tissue into the recipient of step b).

Without being bound by theory, an MHC class I molecule without a pre-loaded molecule in the binding groove (peptide-free MHC class I molecule) may efficiently take up and display native peptides once introduced into the host tissue. Thus, In some embodiments the MHC class I molecule of the present disclosure is peptide-free.

In some embodiments of the present disclosure, the method further comprises a step d) of introducing a peptide specific for said MHC class I molecule of step c) into said transplant tissue prior to or after transplantation of said transplant tissue into said recipient.

In some embodiments of the present disclosure, said peptide is separately expressed from the introduced MHC class I molecule of step c).

In some embodiments of the present disclosure, said peptide is expressed as part of the introduced MHC class I molecule of step c).

In some embodiments of the present disclosure, the MHC class I molecule is an HLA-E molecule. In some embodiments of the present disclosure, the MHC class I molecule is an HLA-F molecule. In some embodiments of the present disclosure, the MHC class I molecule is an HLA-G molecule.

Tissue is to be understood broadly and includes one or more cell lines, one or more types of cells, one or more organs or parts of organs. Examples of tissues are heart, kidney, liver, lung, pancreas, stomach, intestine, cornea, bone, tendon, skin, pancreas islets, heart valves, nerves, veins and CAR-T cells.

In some embodiments of the present disclosure, the tissue is a heart or a part thereof, such as a heart valve. In some embodiments of the present disclosure, the tissue is a kidney or a part thereof. In some embodiments of the present disclosure, the tissue is a liver or a part thereof. In some embodiments of the present disclosure, the tissue is a lung or a part thereof. In some embodiments of the present disclosure, the tissue is a pancreas or a part thereof. In some embodiments of the present disclosure, the tissue is a stomach or a part thereof. In some embodiments of the present disclosure, the tissue is an intestine or a part thereof. In some embodiments of the present disclosure, the tissue is a cornea or a part thereof. In some embodiments of the present disclosure, the tissue is a bone or a part thereof. In some embodiments of the present disclosure, the tissue is a tendon or a part thereof. In some embodiments of the present disclosure, the tissue is skin. In some embodiments of the present disclosure, the tissue is a pancreas or a part thereof, such as a pancreatic islet. In some embodiments of the present disclosure, the tissue is a nerve or a part thereof. In some embodiments of the present disclosure, the tissue is a vein or a part thereof. In some embodiments of the present disclosure, the tissue is a CAR-T cell.

The recipient is in a preferred embodiment a human being. In some embodiments of the present disclosure, the recipient is a non-human animal.

### Nucleic Acid and Stem Cell

In some aspects the present disclosure provides a nucleic acid encoding the MHC class I monomers, MHC class I/peptide monomers, MHC class I multimers and/or MHC class I/peptide multimers according to the present disclosure.

It is also possible to express MHC molecules as single-chain MHC constructs consisting of a peptide-first linker-β₂m-second linker-MHC. Single-chain MHC constructs are further described in Kotsiou et al., 2011. In some embodiments, the present disclosure provides a nucleic acid encoding a single-chain MHC construct comprising the MHC class I monomer or the MHC class I/peptide monomer according to the present disclosure.

In some aspects, the present disclosure provides a cell, such as in a preferred aspect a stem cell, comprising an MHC class I molecule, such as an MHC class I monomer, or an MHC class I/peptide monomer.

In some embodiments, the present disclosure provides a stem cell comprising a single-chain MHC construct comprising the MHC class I monomer or the MHC class I/peptide monomer according to the present disclosure.

In some embodiments of the present disclosure, the MHC class I molecule is an HLA-E molecule. In some embodiments of the present disclosure, the MHC class I molecule is an HLA-F molecule. In some embodiments of the present disclosure, the MHC class I molecule is an HLA-G molecule.

In some embodiments, the MHC class I molecule is peptide-free. In such embodiments, without being bound by theory, the MHC class I molecule without a pre-loaded molecule in the binding groove (peptide-free MHC class I molecule) may efficiently take up and display native peptides once introduced into the host tissue, such as the stem cell.

In other embodiments, the MHC class I molecule comprises a peptide in its binding groove, such as a peptide specific for said MHC class I molecule.

### Items

1. An MHC class I molecule comprising or consisting of at least amino acids 1-51 and 91-137 of the amino acid sequence as set forth in SEQ ID NO: 1 or 2, or a variant of said MHC class I molecule having at least 85% sequence homology or identity thereto, such as at least 90% sequence homology or identity thereto, such as at least 95% sequence homology or identity thereto, or such as at least 98% sequence homology or identity thereto,
   with the proviso that the amino acids corresponding to positions
      i) 4 and 102 of SEQ ID NO: 1 or 2, and/or
      ii) 12 and 94 of SEQ ID NO: 1 or 2, and/or
      iii) 23 and 37 of SEQ ID NO: 1 or 2, and/or
      iv) 115 and 125 of SEQ ID NO: 1 or 2
   have been mutated to cysteines and linked by a disulphide bridge.
2. The MHC class I molecule according to item 1, wherein said MHC class I molecule comprises or consists of at least amino acids 1-180 of the amino acid sequence according to SEQ ID NO: 1 or 2, or a variant of said MHC class I molecule having at least 85% sequence homology or identity thereto, such as at least 90% sequence homology or identity thereto, such as at least 95% sequence homology or identity thereto, or such as at least 98% sequence homology or identity thereto,
   with the proviso that the amino acids corresponding to positions
      i) 4 and 102 of SEQ ID NO: 1 or 2, and/or
      ii) 12 and 94 of SEQ ID NO: 1 or 2, and/or
      iii) 23 and 37 of SEQ ID NO: 1 or 2, and/or
      iv) 115 and 125 of SEQ ID NO: 1 or 2
   have been mutated to cysteines and linked by a disulphide bridge.
3. The MHC class I molecule according to any one of the preceding items, wherein said MHC class I molecule comprises or consists of at least amino acids 1-276 of the amino acid sequence according to SEQ ID NO: 1 or 2, or a variant of said MHC class I molecule having at least 85% sequence homology or identity thereto, such as at least 90% sequence homology or identity thereto, such as at least 95% sequence homology or identity thereto, or such as at least 98% sequence homology or identity thereto,
   with the proviso that the amino acids corresponding to positions
      i) 4 and 102 of SEQ ID NO: 1 or 2, and/or
      ii) 12 and 94 of SEQ ID NO: 1 or 2, and/or
      iii) 23 and 37 of SEQ ID NO: 1 or 2, and/or
      iv) 115 and 125 of SEQ ID NO: 1 or 2
   have been mutated to cysteines and linked by a disulphide bridge.
4. An MHC class I molecule comprising or consisting of at least amino acids 91-276 of the amino acid sequence according to SEQ ID NO: 1 or 2, or a variant of said MHC class I molecule having at least 85% sequence homology or identity thereto, such as at least 90% sequence homology or identity thereto, such as at least 95% sequence homology or identity thereto, or such as at least 98% sequence homology or identity thereto,
   with the proviso that the amino acids corresponding to positions
      i) 115 and 125 of SEQ ID NO: 1 or 2
   have been mutated to cysteines and linked by a disulphide bridge.
5. The MHC class I molecule according to any one of the preceding items, wherein said variant of said MHC class I molecule has at least 95% sequence homology or identity thereto.
6. The MHC class I molecule according to any one of the preceding items, wherein said MHC class I molecule comprises or consists of
   a. the amino acid sequence as set forth in SEQ ID NO: 3 or SEQ ID NO: 4, or comprises or consists of a variant of said MHC class I molecule having at least 85% sequence homology or identity thereto, such as at least 90% sequence homology or identity thereto, such as at least 95% sequence homology or identity thereto, or such as at least 98% sequence homology or identity thereto,
      with the proviso that the amino acids corresponding to positions 4 and 102 of SEQ ID NO: 3 or 4 are cysteines and linked by a disulphide bridge; or
   b. the amino acid sequence as set forth in SEQ ID NO: 5 or SEQ ID NO: 6, or comprises or consists of a variant of said MHC class I molecule having at least 85% sequence homology or identity thereto, such as at least 90% sequence homology or identity thereto, such as at least 95% sequence homology or identity thereto, or such as at least 98% sequence homology or identity thereto,
      with the proviso that the amino acids corresponding to positions 12 and 94 of SEQ ID NO: 5 or 6 are cysteines and linked by a disulphide bridge; or
   c. the amino acid sequence as set forth in SEQ ID NO: 7 or SEQ ID NO: 8, or comprises or consists of a variant of said MHC class I molecule having at least 85% sequence homology or identity thereto, such as at least 90% sequence homology or identity thereto, such as at least 95% sequence homology or identity thereto, or such as at least 98% sequence homology or identity thereto,
      with the proviso that the amino acids corresponding to positions 115 and 125 of SEQ ID NO: 7 or 8 are cysteines and linked by a disulphide bridge; or
   d. the amino acid sequence as set forth in SEQ ID NO: 9 or SEQ ID NO: 10, or comprises or consists of a variant of said MHC class I molecule having at least 85% sequence homology or identity thereto, such as at least 90% sequence homology or identity thereto, such as at least 95% sequence homology or identity thereto, or such as at least 98% sequence homology or identity thereto,
      with the proviso that the amino acids corresponding to positions 23 and 37 of SEQ ID NO: 9 or 10 are cysteines and linked by a disulphide bridge.
7. The MHC class I molecule according to any one of the preceding items, wherein the MHC class I molecule has increased stability, such as an increased melting temperature,
   such as wherein the MHC class I molecule has a melting temperature of at least 49.0° C., such as at least 50.0° C., such as at least 51.0° C., such as at least 52.0° C., such as at least 53.0° C., such as at least 54.0° C., such as at least 55.0° C., or such as at least 56.0° C., and/or
   such as wherein the MHC class I molecule has an increased melting temperature of at least 1.0° C., such as at least 1.25° C., such as at least 1.5° C., such as at least 2.0° C., such as at least 2.5° C., such as at least 3.0° C., such as at least 4.0° C., such as at least 5.0° C. or such as at least 6.0° C., compared to a corresponding wild type MHC class I molecule of identical sequence except not comprising said cysteine mutations linked by a disulphide bridge.
8. The MHC class I molecule according to any one of the preceding items, wherein the MHC class I molecule comprises a refolding test peptide in its peptide-binding groove and has an increased refolding yield compared to a corresponding wild type MHC class I molecule of identical sequence except not comprising said cysteine mutations linked by a disulphide bridge, said corresponding wild type MHC class I molecule also comprising said refolding test peptide in its peptide-binding groove,
   such as wherein said increased refolding yield is a refolding yield of at least 125%, such as at least 150%, such as at least 175%, such as at least 200%, such as at least 250%, such as at least 300%, such as at least 350%, such as at least 400%, or such as at least 450% compared to said corresponding wild type MHC class I molecule.
9. The MHC class I molecule according to any one of the preceding items, wherein the MHC class I molecule is selected from the group consisting of HLA-A, HLA-B, HLA-C, HLA-E, HLA-F, HLA-G, MR1 and CD1.
10. The MHC class I molecule according to any one of the preceding items, wherein the MHC class I molecule is HLA-E.
11. An MHC class I multimer or an MHC class I/peptide multimer comprising at least two MHC class I monomers according to any one of the preceding items.
12. A cell, such as a stem cell, comprising an MHC class I molecule, an MHC class I multimer and/or an MHC class I/peptide multimer according to any one of the preceding items.
13. A method for isolation of one or more immune cells, such as antigen-specific T cells, said method comprising the steps of:
   a) providing a sample comprising a population of immune cells, such as T cells;
   b) providing one or more MHC class I monomers, MHC class I/peptide monomers, MHC class I multimers and/or MHC class I/peptide multimers according to any one of items 1 to 11, or a composition comprising one or more of said MHC class I monomers, MHC class I/peptide monomers, MHC class I multimers and/or MHC class I/peptide multimers;
   c) contacting said MHC class I monomers, MHC class I/peptide monomers, MHC class I multimers and/or MHC class I/peptide multimers, or said composition, with said sample; and
   d) isolating said immune cells, such as T cells specific for said MHC class I monomers, MHC class I/peptide monomers, MHC class I multimers and/or MHC class I/peptide multimers, or composition.
14. A method for detecting an immune cell response, such as an antigen-specific T cell response, comprising the steps of:
   a) providing a sample comprising a population of immune cells, such as T cells;
   b) providing one or more MHC class I monomers, MHC class I/peptide monomers, MHC class I multimers and/or MHC class I/peptide multimers according to any one of items 1 to 11, or a composition comprising one or more of said MHC class I monomers, MHC class I/peptide monomers, MHC class I multimers and/or MHC class I/peptide multimers;
   c) contacting said MHC class I monomers, MHC class I/peptide monomers, MHC class I multimers and/or MHC class I/peptide multimers, or said composition, with said sample; and
   d) measuring the presence, frequency, number, activity and/or state of immune cells, such as T cells specific for said MHC class I monomers, MHC class I/peptide monomers, MHC class I multimers and/or MHC class I/peptide multimers, or composition,
   thereby detecting said immune cell response, such as said antigen-specific T cell response.
15. An MHC class I monomer, an MHC class I/peptide monomer, an MHC class I multimer, or an MHC class I/peptide multimer, according to any one of items 1 to 11, or a combination thereof, for use in a method of limiting or preventing NK cell cytotoxicity during or following tissue transplantation of a transplant tissue into a recipient, such as a human being.

### EXAMPLES

### Example 1—Production of HLA-E Monomers

The HLA-E heavy (HC) and light (β₂m) chains were produced separately and partially purified as inclusion bodies from an E. coli strain (BL21 (DE3); Novagen, Madison, WI, USA) following standard procedure. The HLA-E HC consisted of the first 276 residues of the native protein sequence (SEQ ID NO: 2) and an added, C-terminal tag for enzymatic biotinylation (SEQ ID NO: 11), and thus omits the signal peptide, transmembrane region, and cytosolic domain.

The isolated inclusion body molecules were solubilised in 8M urea to obtain HC and β₂m preparations for subsequent refolding. The β₂m molecule was refolded by changing to a urea-free buffer and was subsequently purified by size exclusion chromatography following standard procedure.

The HC molecule was additionally purified by ion-exchange chromatography following standard procedure and finally subjected to folding as described below. Peptide epitope-specific HLA-E complexes were generated in vitro using a “folding by dilution” approach where the highly purified preparations of denatured HLA-E heavy chain molecule (about 10-20 μM in 8M urea) were renatured by incubation in a 100-fold dilution buffer (final concentration of heavy chain is thus about 100-200 nM) containing the peptide of interest (10 μM) and folded β₂m (1 μM), for 16 hours at 10° C. Next, the de novo-folded HLA-E molecule was buffer-changed and biotinylated using birA biotin ligase as described by the manufacturer (AVIDITY; Denver, CO, USA). Finally, the mono-biotinylated HLA-E complex was purified by size exclusion chromatography following standard procedure.

### Example 2—Refolding Yields

The refolding yields for different peptide and HLA-E variant combinations produced as described in example 1 were evaluated. Assuming that all HC added to the refolding mix can form MHC complexes with peptide and β2m, the theoretical yield of a given refolding reaction can be calculated. The actual yield for the present peptide and HLA-E variant combinations was measured as the absorbance at 280 nm of the final, purified MHC complex solution. The A280 was transformed into a protein concentration and then a total yield. Comparing this actual yield with the theoretical yield gave the relative yields.

This revealed surprising and significant improvements over WT HLA-E for several of the variants when refolded with inhA peptide (RLPAKAPLL—SEQ ID NO: 12). An increase in yield is evident for HLA-E variants with the following mutations: S4C/E102C (SEQ ID NO: 4), V12C/T94C (SEQ ID NO: 6), 123C/D37C (SEQ ID NO: 10), Q115C/T125C (SEQ ID NO: 8), and V12C/T94C/Q115C/T125C (SEQ ID NO: 17). As an illustration of the role of the refolding peptide, the UL40 peptide (VMAPRTLIL—SEQ ID NO: 13) only gave increased yield with the V12C/T94C variant.

Although the HLA-E variants are designed using the same principle of introducing cysteine residues at positions in the HLA-E sequence judged to be close enough to form a disulphide bridge in the folded HLA-E molecule, not all variants exhibited improved refolding yield. Specifically, W133C/E144C, which borrows a native disulphide bridge observed in the non-classical MHC-I molecule CD1b (Gadola et al., 2002) exhibits lower yields than WT HLA-E. Unexpectedly, the effects of individual mutation pairs are not additive as evidenced by a drop in refolding yield with the V12C/T94C/Q115C/T125C relative to the V12C/T94C variant, even though the Q115C/T125C variant itself exhibits increased refolding yield.

In short, introducing disulphide bridges in HLA-E to increase the yield is non-trivial and the effect of such disulphide bridges is further dependent on the refolding peptide. The refolding yields for various combinations of refolding peptide and HLA-E variant are summarised in table 2, below.

TABLE 2
Refolding yields

| | Refolding yield | | Refolding yield | |
|---|---|---|---|---|
| | with inhA peptide | | with UL40 peptide | |
| | Relative to | | Relative to | |
| | theoretical | Relative to | theoretical | Relative to |
| HLA-E Variant | max; % | WT; % | max; % | WT; % |
| WT | 8 | 100 | 25 | 100 |
| S4C/E102C | 16 | 208 | 14 | 57 |
| V12C/T94C | 36 | 464 | 32 | 131 |
| I23C/D37C | 16 | 198 | 12 | 48 |
| Q115C/T125C | 11 | 144 | 11 | 47 |
| W133C/E144C | 5 | 66 | 12 | 49 |
| V12C/T94C/ | 18 | 231 | 14 | 56 |
| Q115C/T125C | | | | |

### Example 3—Stability Testing of HLA-E Variants

To investigate the stabilising effect of introducing disulphide bridges in HLA-E, the variants in table 2 were refolded with inhA peptide and subjected to stability analysis using thermal shift analysis through nanoscale differential scanning fluorimetry (nanoDSF). Briefly, HLA-E monomer samples were diluted to a concentration of 0.2 mg/mL in a buffer containing 50 mM NaCl and 20 mM Tris-HCl pH 8.0, transferred to Prometheus® High Sensitivity Capillaries and placed into a Prometheus® NT.48 nanoDSF system. Thermal denaturation was performed by increasing the temperature from 20° C. to 95° C. at a rate of 1° C./minute, and the melting temperature for each sample was determined from the first derivative of the 350 nm/330 nm ratio (d[F350/F330]/dT) as described by the manufacturer (NanoTemper Technologies GmbH, Munich, Germany).

The results of this assay are shown in table 3, below.

TABLE 3
Stability of HLA-E variants

NanoDSF

| HLA-E variant w. | Tm; | Stdev; | Tm diff; |
|---|---|---|---|
| inhA peptide | ° C. | ° C. | ° C. |
| WT | 47.5 | 0.1 | 0.0 |
| S4C/E102C | 51.4 | 0.0 | 3.9 |
| V12C/T94C | 49.1 | 0.1 | 1.6 |
| I23C/D37C | 48.1 | 0.1 | 0.6 |
| Q115C/T125C | 54.9 | 0.5 | 7.4 |
| W133C/E144C | 38.8 | 0.2 | −8.7 |
| V12C/T94C/Q115C/T125C | 49.9 | 0.2 | 2.5 |

The introduction of non-native disulphide bridges in HLA-E exhibits a wide range of effects on HLA-E peptide complexes ranging from severe loss of stability (decreased melting point, Tm) to significant improvements (increased Tm). Surprisingly, changes in stability are not correlated with improvements in refolding yield. In particular, the introduction of a native disulphide bridge observed in the non-classical MHC-I molecule CD1b (W133C/E144C) leads to a pronounced loss of complex stability. As was observed for refolding yields, the stabilizing effects observed for individual HLA-E disulphide variants are not additive as evidence by the modest increase in stability of the V12C/T94C/Q115C/T125C variant over the V12C/T94C variant. In summary, the effect of introducing non-native disulphide bridges in HLA-E is surprisingly complex and unpredictable with only some variants exhibiting significant and useful improvements in stability.

This thus demonstrates that the position of the disulphide is non-trivial as evidenced by the failure of many of such disulphide bridges to contribute significant stability to HLA-E complexes.

### Example 4—Initial Functional Validation—TCR Specificity Assay

The modification of an MHC molecule may disrupt the native conformation of the complex and thus perturb its interaction with TCR molecules or other MHC-engaging receptors.

To investigate the impact of HLA-E modifications on TCR recognition, all HLA-E variants were refolded with inhA peptide and tested for binding to an HLA-E/inhA-specific TCR (inhA:01 TCR; Barber & Arena de Souza et al., 2022) as well as an unrelated TCR (1G4_c58c61; Sami et al., 2007) following a previously described method (Low et al., 2012). Briefly, for each HLA-E variant, SPHERO™ Streptavidin magnetic beads (Spherotech, Lake Forest, IL, USA) were coated with corresponding HLA-E/inhA complexes, and the beads were subsequently stained with phycoerythrin-labelled TCR multimers to demonstrate native-like recognition of the HLA-E/inhA variants by a TCR. TCR multimer reagents were prepared as Dextramer® reagents labelled with phycoerythrin fluorophores as described previously for MHC Dextramer® reagents (Example 1 in WO/2002/072631). After staining and washing, the TCR Dextramer®-stained HLA-E beads were analysed by flow cytometry on a NovoCyte flow cytometer (Agilent Technologies, Santa Clara, CA, USA) following standard procedure.

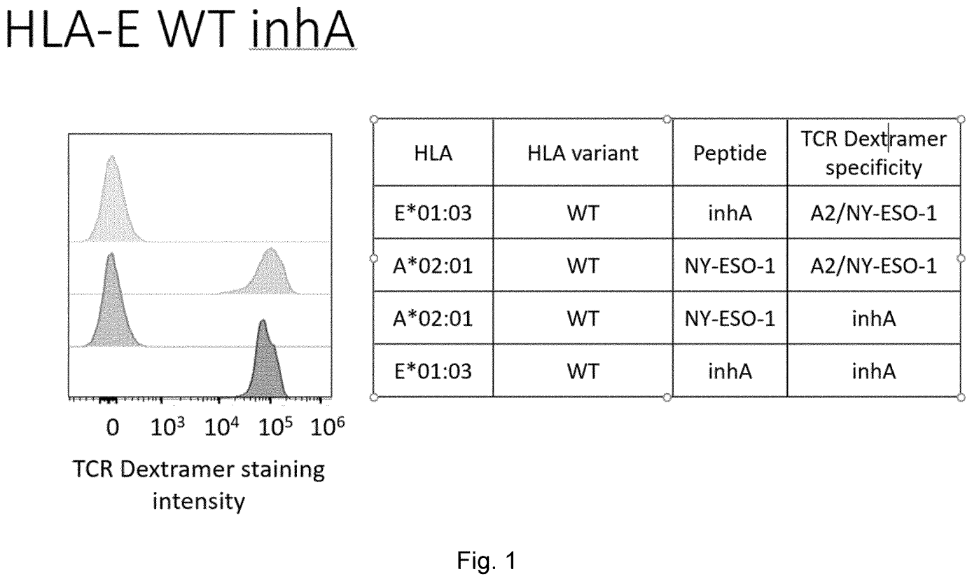

For each HLA-E variant, specific recognition by the HLA-E/inhA-specific TCR was monitored by the fluorescence intensity of the corresponding TCR Dextramer-stained HLA-E beads. To control for any unspecific binding, HLA-E beads were also stained with an unrelated TCR Dextramer® specific for HLA-A*02:01 in complex with NY-ESO-1 peptide (SLLMWITQC—SEQ ID NO: 14). FIG. 1 shows histograms from the flow cytometry analysis of TCR Dextramer®-stained HLA-E beads for HLA-E*01:03 wild type.

The bead-based flow cytometry analysis confirms that all HLA-E variants in complex with the inhA peptide, except W133C/E144C, exhibit native-like recognition by the HLA-E/inhA-specific TCR. No unspecific TCR binding was observed for any of the HLA-E variants or TCR Dextramer® reagents. The results of testing HLA-E variants with TCR Dextramer® reagents are summarised in table 4; Variants marked with “+” exhibit specific staining of pHLA-E beads with the corresponding TCR Dextramer® reagent; variants marked with “−” exhibit no staining.

TABLE 4
HLA-E variant recognition by TCRs

| | | Recognition by TCR | |
|---|---|---|---|
| | | Dextramer reagent | |
| | | TCR | TCR |
| | Pep- | specificity: | specificity: |
| HLA-E*01:03 Variants | tide | HLA-E/inhA | HLA-A2/NY-ESO-1 |
| WT | inhA | + | − |
| S4C/E102C | inhA | + | − |
| V12C/T94C | inhA | + | − |
| I23C/D37C | inhA | + | − |
| Q115C/T125C | inhA | + | − |
| W133C/E144C | inhA | − | − |
| V12C/T94C/Q115C/T125C | inhA | + | − |

### Example 5—Functional Testing of HLA-E Variant Complexes on PBMC Samples—Specific Staining of NK Cells

HLA-E in complex with HLA signal peptide fragments is a known target of the CD94/NKG2 receptor expressed at the surface of NK and NKT cells, and thus HLA-E multimer reagents can be used to specifically stain NK and NKT cells in analogy with staining of antigen-specific T cells using classical MHC multimer reagents. Here, we tested whether the HLA-E variant molecules retained their native conformation in the context of NK and NKT cell recognition by staining samples of peripheral blood mononuclear cells (PMBC) with HLA-E Dextramer reagents loaded with UL40 peptide (residues 15-23, sequence: VMAPRTLIL—SEQ ID NO: 13). The UL40 peptide is derived from cytomegalovirus, is identical to the signal peptide fragment of HLA-Cw*01, is highly similar to other HLA-A/B/C signal peptide fragments and is a target for NK cells.

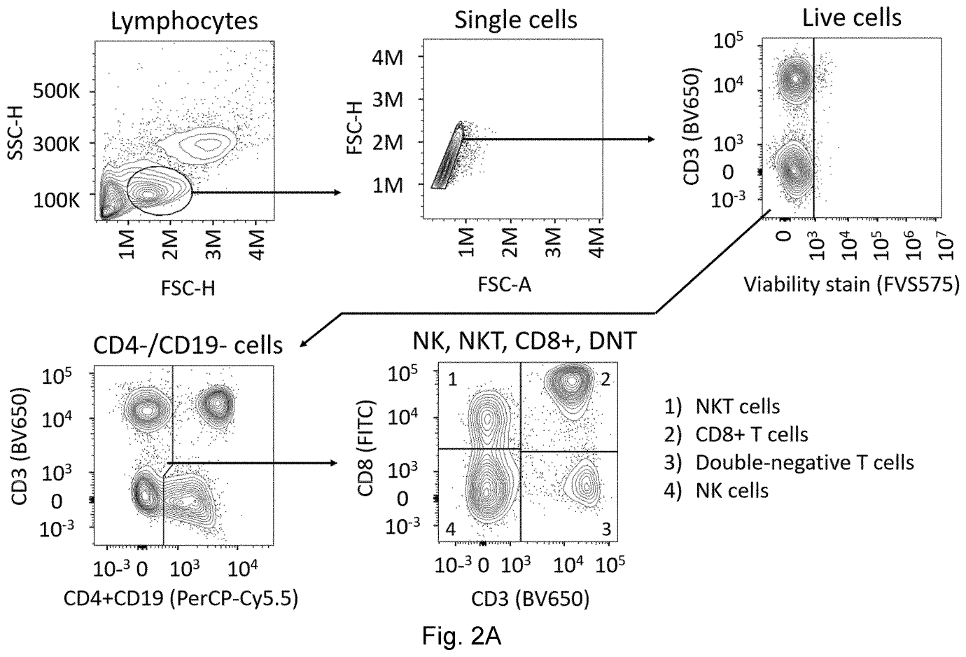

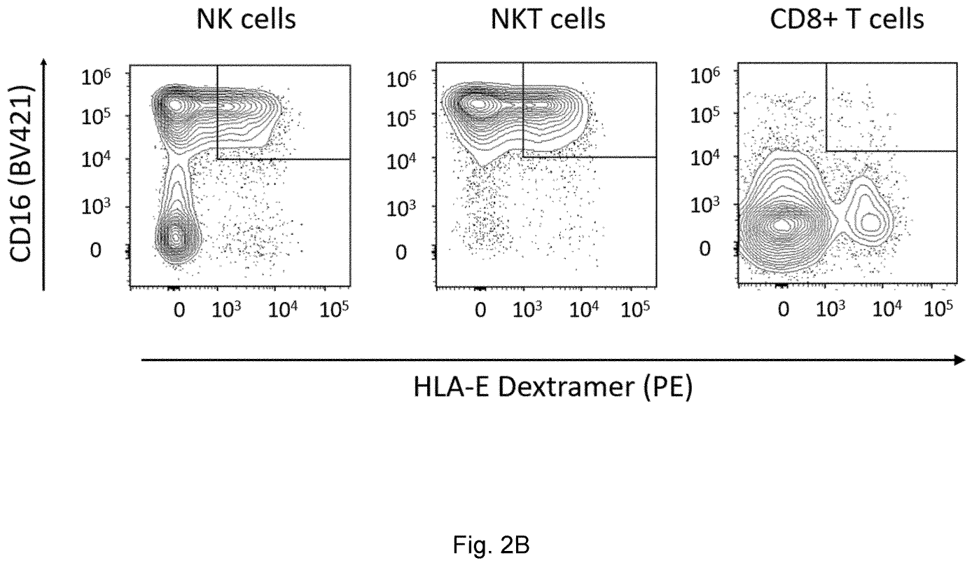

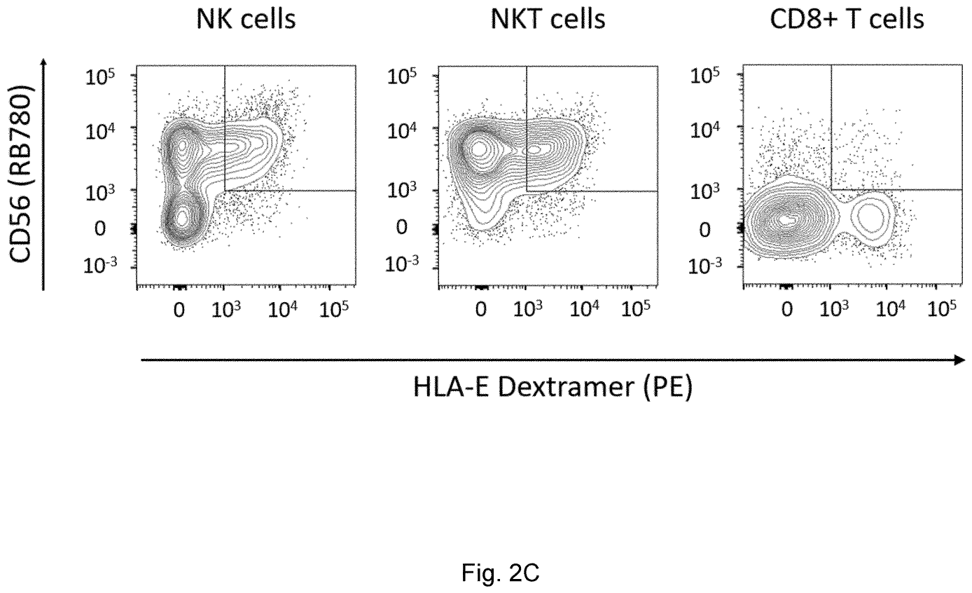

Samples of PBMCs were stained individually with an UL40/HLA-E Dextramer® reagent for each HLA-E variant in combination with an antibody panel (see table 5) following standard procedure. Briefly, for each HLA-E variant, a PBMC sample containing 800,000 cells in 50 μL PBS with 5% serum was mixed with 10 μL (32 nM) HLA-E Dextramer® and 40 μL antibody mixture following standard procedure. HLA-E Dextramer reagents labelled with phycoerythrin fluorophores were prepared as described previously (Example 1 in WO/2002/072631). After staining and washing, the PBMCs were analysed by flow cytometry on a NovoCyte flow cytometer (Agilent Technologies, Santa Clara, CA, USA) following standard procedure. Single, live lymphocytes were gated based on forward scatter (FSC) and side scatter (SSC) profiles as well as staining with viability dye as illustrated in FIG. 2. From these, CD4-/CD19−cells were selected and further divided in three subsets based on their expression of CD3 and CD8. NK cells are defined as CD3^{low}CD8^{low}, NKT cells as CD3^{low}CD8^{high} and CD8+ T cells as CD3^{high}CD8^{high}. Within each of these three subsets, we selected cells which simultaneously stained with HLA-E Dextramer® reagent and exhibited high levels of expression of one of the two NK cell-specific surface markers CD16 or CD56− see FIG. 2 for gating strategy and example data with UL40/HLA-E*01:03 wild type Dextramer® reagents. The fraction of HLA-E+/CD16+ and HLA-E+/CD56+ out of all CD4−/CD19− lymphocytes for NK cells, NKT cells and CD8+ T cells, respectively, is summarised in table 6. HLA-A*02:01 carrying a negative control peptide (peptide sequence: ALIAPVHAV—SEQ ID NO: 15) was used to test for unspecific binding of HLA molecules.

TABLE 5
Antibody panel for staining of PBMCs

| Specificity | Fluorochrome |
|---|---|
| Viability stain | FVS575 |
| CD3 | BV650 |
| CD4 | PerCP-Cy5.5 |
| CD8 | FITC |
| CD16 | BV421 |
| CD19 | PerCP-Cy5.5 |
| CD56 | RB780 |

Staining of PBMC samples demonstrate native-like recognition of several HLA-E variants in complex with the UL40/HLA-Cw01 leader peptide, specifically S4C/E102C, V12C/T94C, 123C/D37C, and Q115C/T125C. In agreement with stability measurements and functional validation by TCR binding, the W133C/E144C variant is not recognised by NK or NKT cells. Surprisingly, the V12C/T94C/Q115C/T125C variant is not recognised by T cells although it does bind the test TCR (see example 4). CD8+ T cells only exhibit very low levels of staining with HLA-E reagents, and no cells exhibit unspecific HLA-staining as tested by the HLA-A*02:01 negative control Dextramer® reagent.

The results for staining of CD4−/CD19− lymphocytes with HLA-E Dextramer® reagents are summarised in table 6, below. Fields marked with “+” signify specific staining of >8% of the parent cell population (NK cells, NKT cells or CD8+ T cells) with the corresponding HLA-E Dextramer® reagent, while fields marked with “−” correspond to staining of <1% (CD16) or <2% (CD56) of the parent cell population.

TABLE 6

| | | NK cells | | NKT cells | | CD8+ T cells | |
|---|---|---|---|---|---|---|---|
| MHC Variants | Peptide | CD16+ | CD56+ | CD16+ | CD56+ | CD16+ | CD56+ |
| HLA-A*02:01 | Neg. Ctrl. | − | − | − | − | − | − |
| pHLA-E*01:03 (WT) | UL40 | + | + | + | + | − | − |
| pHLA-E S4C/E102C | UL40 | + | + | + | + | − | − |
| pHLA-E V12C/T94C | UL40 | + | + | + | + | − | − |
| pHLA-E I23C/D37C | UL40 | + | + | + | + | − | − |
| pHLA-E Q115C/T125C | UL40 | + | + | + | + | − | − |
| pHLA-E W133C/E144C | UL40 | − | − | − | − | − | − |
| pHLA-E V12C/T94C/ | UL40 | − | − | − | − | − | − |
| Q115C/T125C | | | | | | | |

This thus demonstrates that the position of the disulphide is non-trivial as evidenced by the failure of several of such disulphide bridges to retain native-like recognition of the HLA-E by a T cell receptor or NK cell.

### Example 6—In Vivo/Ex Vivo Applications: Cell-Based Assays and Systems, Including Cellular Therapy, Immunotherapy, and Organ/Stem Cell Transplantation

A stabilised form of HLA-E allows for higher surface expression levels of HLA-E complexes, which may find use in technical applications such as cell-based assays for the study of HLA-E ligands. Stabilised HLA-E molecules with higher surface expression levels may also prevent NK cell cytotoxicity and may be exploited in the context of tissue transplants or adoptive cell therapy. Eliminating expression of native MHC expression in a transplant can prevent cells or tissues from being rejected by the adaptive immune system of the recipient (e.g. by using a β₂m knockout). To simultaneously prevent NK cell cytotoxicity, HLA-E can be reintroduced into the transplanted tissue, e.g. as a single-chain construct (typically β₂m-linker-HC) possibly combined with an additional source of relevant peptides such as HLA leader peptides (either separately expressed or expressed as part of the introduced HLA-E protein).

Using a stabilised form of HLA-E allows for higher surface expression levels of HLA-E complexes, which increases the ability to pacify NK cells and thus prevents tissue rejection.

Sequence overview

| SEQ ID | | | |
|---|---|---|---|
| NO: | Description | Organism | Sequence |
| SEQ ID | HLA-E*01:01; | Homo | GSHSLKYFHT SVSRPGRGEP |
| NO: 1 | position 1-276 of | sapiens | RFISVGYVDD TQFVRFDNDA |
| | the native protein | | ASPRMVPRAP WMEQEGSEYW |
| | sequence | | DRETRSARDT AQIFRVNLRT |
| | | | LRGYYNQSEA GSHTLQWMHG |
| | | | CELGPDRRFL RGYEQFAYDG |
| | | | KDYLTLNEDL RSWTAVDTAA |
| | | | QISEQKSNDA SEAEHQRAYL |
| | | | EDTCVEWLHK YLEKGKETLL |
| | | | HLEPPKTHVT HHPISDHEAT |
| | | | LRCWALGFYP AEITLTWQQD |
| | | | GEGHTQDTEL VETRPAGDGT |
| | | | FQKWAAVVVP SGEEQRYTCH |
| | | | VQHEGLPEPV TLRWKP |
| SEQ ID | HLA-E*01:03; | Homo | GSHSLKYFHT SVSRPGRGEP |
| NO: 2 | position 1-276 of | sapiens | RFISVGYVDD TQFVRFDNDA |
| | the native protein | | ASPRMVPRAP WMEQEGSEYW |
| | sequence | | DRETRSARDT AQIFRVNLRT |
| | | | LRGYYNQSEA GSHTLQWMHG |
| | | | CELGPDGRFL RGYEQFAYDG |
| | | | KDYLTLNEDL RSWTAVDTAA |
| | | | QISEQKSNDA SEAEHQRAYL |
| | | | EDTCVEWLHK YLEKGKETLL |
| | | | HLEPPKTHVT HHPISDHEAT |
| | | | LRCWALGFYP AEITLTWQQD |
| | | | GEGHTQDTEL VETRPAGDGT |
| | | | FQKWAAVVVP SGEEQRYTCH |
| | | | VQHEGLPEPV TLRWKP |
| SEQ ID | HLA-E*01:01_1- | Synthetic | GSHCLKYFHT SVSRPGRGEP |
| NO: 3 | 276_S4C_E102C | construct | RFISVGYVDD TQFVRFDNDA |
| | | | ASPRMVPRAP WMEQEGSEYW |
| | | | DRETRSARDT AQIFRVNLRT |
| | | | LRGYYNQSEA GSHTLQWMHG |
| | | | CCLGPDRRFL RGYEQFAYDG |
| | | | KDYLTLNEDL RSWTAVDTAA |
| | | | QISEQKSNDA SEAEHQRAYL |
| | | | EDTCVEWLHK YLEKGKETLL |
| | | | HLEPPKTHVT HHPISDHEAT |
| | | | LRCWALGFYP AEITLTWQQD |
| | | | GEGHTQDTEL VETRPAGDGT |
| | | | FQKWAAVVVP SGEEQRYTCH |
| | | | VQHEGLPEPV TLRWKP |
| SEQ ID | HLA-E*01:03_1- | Synthetic | GSHCLKYFHT SVSRPGRGEP |
| NO: 4 | 276_S4C_E102C | construct | RFISVGYVDD TQFVRFDNDA |
| | | | ASPRMVPRAP WMEQEGSEYW |
| | | | DRETRSARDT AQIFRVNLRT |
| | | | LRGYYNQSEA GSHTLQWMHG |
| | | | CCLGPDGRFL RGYEQFAYDG |
| | | | KDYLTLNEDL RSWTAVDTAA |
| | | | QISEQKSNDA SEAEHQRAYL |
| | | | EDTCVEWLHK YLEKGKETLL |
| | | | HLEPPKTHVT HHPISDHEAT |
| | | | LRCWALGFYP AEITLTWQQD |
| | | | GEGHTQDTEL VETRPAGDGT |
| | | | FQKWAAVVVP SGEEQRYTCH |
| | | | VQHEGLPEPV TLRWKP |
| SEQ ID | HLA- | Synthetic | GSHSLKYFHT SCSRPGRGEP |
| NO: 5 | E*01:01_V12C- | construct | RFISVGYVDD TQFVRFDNDA |
| | T94C_1-276 | | ASPRMVPRAP WMEQEGSEYW |
| | | | DRETRSARDT AQIFRVNLRT |
| | | | LRGYYNQSEA GSHCLQWMHG |
| | | | CELGPDRRFL RGYEQFAYDG |
| | | | KDYLTLNEDL RSWTAVDTAA |
| | | | QISEQKSNDA SEAEHQRAYL |
| | | | EDTCVEWLHK YLEKGKETLL |
| | | | HLEPPKTHVT HHPISDHEAT |
| | | | LRCWALGFYP AEITLTWQQD |
| | | | GEGHTQDTEL VETRPAGDGT |
| | | | FQKWAAVVVP SGEEQRYTCH |
| | | | VQHEGLPEPV TLRWKP |
| SEQ ID | HLA- | Synthetic | GSHSLKYFHT SCSRPGRGEP |
| NO: 6 | E*01:03_V12C- | construct | RFISVGYVDD TQFVRFDNDA |
| | T94C_1-276 | | ASPRMVPRAP WMEQEGSEYW |
| | | | DRETRSARDT AQIFRVNLRT |
| | | | LRGYYNQSEA GSHCLQWMHG |
| | | | CELGPDGRFL RGYEQFAYDG |
| | | | KDYLTLNEDL RSWTAVDTAA |
| | | | QISEQKSNDA SEAEHQRAYL |
| | | | EDTCVEWLHK YLEKGKETLL |
| | | | HLEPPKTHVT HHPISDHEAT |
| | | | LRCWALGFYP AEITLTWQQD |
| | | | GEGHTQDTEL VETRPAGDGT |
| | | | FQKWAAVVVP SGEEQRYTCH |
| | | | VQHEGLPEPV TLRWKP |
| SEQ ID | HLA-E*01:01 | Synthetic | GSHSLKYFHT SVSRPGRGEP |
| NO: 7 | Q115C- | construct | RFISVGYVDD TQFVRFDNDA |
| | T125C_1-276 | | ASPRMVPRAP WMEQEGSEYW |
| | | | DRETRSARDT AQIFRVNLRT |
| | | | LRGYYNQSEA GSHTLQWMHG |
| | | | CELGPDRRFL RGYECFAYDG |
| | | | KDYLCLNEDL RSWTAVDTAA |
| | | | QISEQKSNDA SEAEHQRAYL |
| | | | EDTCVEWLHK YLEKGKETLL |
| | | | HLEPPKTHVT HHPISDHEAT |
| | | | LRCWALGFYP AEITLTWQQD |
| | | | GEGHTQDTEL VETRPAGDGT |
| | | | FQKWAAVVVP SGEEQRYTCH |
| | | | VQHEGLPEPV TLRWKP |
| SEQ ID | HLA-E*01:03 | Synthetic | GSHSLKYFHT SVSRPGRGEP |
| NO: 8 | Q115C- | construct | RFISVGYVDD TQFVRFDNDA |
| | T125C_1-276 | | ASPRMVPRAP WMEQEGSEYW |
| | | | DRETRSARDT AQIFRVNLRT |
| | | | LRGYYNQSEA GSHTLQWMHG |
| | | | CELGPDGRFL RGYECFAYDG |
| | | | KDYLCLNEDL RSWTAVDTAA |
| | | | QISEQKSNDA SEAEHQRAYL |
| | | | EDTCVEWLHK YLEKGKETLL |
| | | | HLEPPKTHVT HHPISDHEAT |
| | | | LRCWALGFYP AEITLTWQQD |
| | | | GEGHTQDTEL VETRPAGDGT |
| | | | FQKWAAVVVP SGEEQRYTCH |
| | | | VQHEGLPEPV TLRWKP |
| SEQ ID | HLA- | Synthetic | GSHSLKYFHT SVSRPGRGEP |
| NO: 9 | E*01:01_I23C- | construct | RFCSVGYVDD TQFVRFCNDA |
| | D37C_1-276 | | ASPRMVPRAP WMEQEGSEYW |
| | | | DRETRSARDT AQIFRVNLRT |
| | | | LRGYYNQSEA GSHTLQWMHG |
| | | | CELGPDRRFL RGYEQFAYDG |
| | | | KDYLTLNEDL RSWTAVDTAA |
| | | | QISEQKSNDA SEAEHQRAYL |
| | | | EDTCVEWLHK YLEKGKETLL |
| | | | HLEPPKTHVT HHPISDHEAT |
| | | | LRCWALGFYP AEITLTWQQD |
| | | | GEGHTQDTEL VETRPAGDGT |
| | | | FQKWAAVVVP SGEEQRYTCH |
| | | | VQHEGLPEPV TLRWKPGSGS |
| SEQ ID | HLA- | Synthetic | GSHSLKYFHT SVSRPGRGEP |
| NO: 10 | E*01:03_I23C- | construct | RFCSVGYVDD TQFVRFCNDA |
| | D37C_1-276 | | ASPRMVPRAP WMEQEGSEYW |
| | | | DRETRSARDT AQIFRVNLRT |
| | | | LRGYYNQSEA GSHTLQWMHG |
| | | | CELGPDGRFL RGYEQFAYDG |
| | | | KDYLTLNEDL RSWTAVDTAA |
| | | | QISEQKSNDA SEAEHQRAYL |
| | | | EDTCVEWLHK YLEKGKETLL |
| | | | HLEPPKTHVT HHPISDHEAT |
| | | | LRCWALGFYP AEITLTWQQD |
| | | | GEGHTQDTEL VETRPAGDGT |
| | | | FQKWAAVVVP SGEEQRYTCH |
| | | | VQHEGLPEPV TLRWKP |
| SEQ ID | Avitag | Synthetic | GSGSLH HILDAQKMVW NHR |
| NO: 11 | | construct | |
| SEQ ID | inhA | Synthetic | RLPAKAPLL |
| NO: 12 | | construct | |
| SEQ ID | UL40 | Synthetic | VMAPRTLIL |
| NO: 13 | | construct | |
| SEQ ID | NY-ESO-1 | Synthetic | SLLMWITQC |
| NO: 14 | | construct | |
| SEQ ID | Negative control | Synthetic | ALIAPVHAV |
| NO: 15 | peptide | construct | |
| SEQ ID | HLA-E*01:01 | Synthetic | GSHSLKYFHT SCSRPGRGEP |
| NO: 16 | V12C-T94C- | construct | RFISVGYVDD TQFVRFDNDA |
| | Q115C- | | ASPRMVPRAP WMEQEGSEYW |
| | T125C_1-276 | | DRETRSARDT AQIFRVNLRT |
| | | | LRGYYNQSEA GSHCLQWMHG |
| | | | CELGPDRRFL RGYECFAYDG |
| | | | KDYLCLNEDL RSWTAVDTAA |
| | | | QISEQKSNDA SEAEHQRAYL |
| | | | EDTCVEWLHK YLEKGKETLL |
| | | | HLEPPKTHVT HHPISDHEAT |
| | | | LRCWALGFYP AEITLTWQQD |
| | | | GEGHTQDTEL VETRPAGDGT |
| | | | FQKWAAVVVP SGEEQRYTCH |
| | | | VQHEGLPEPV TLRWKP |
| SEQ ID | HLA-E*01:03 | Synthetic | GSHSLKYFHT SCSRPGRGEP |
| NO: 17 | V12C-T94C- | construct | RFISVGYVDD TQFVRFDNDA |
| | Q115C- | | ASPRMVPRAP WMEQEGSEYW |
| | T125C_1-276 | | DRETRSARDT AQIFRVNLRT |
| | | | LRGYYNQSEA GSHCLQWMHG |
| | | | CELGPDGRFL RGYECFAYDG |
| | | | KDYLCLNEDL RSWTAVDTAA |
| | | | QISEQKSNDA SEAEHQRAYL |
| | | | EDTCVEWLHK YLEKGKETLL |
| | | | HLEPPKTHVT HHPISDHEAT |
| | | | LRCWALGFYP AEITLTWQQD |
| | | | GEGHTQDTEL VETRPAGDGT |
| | | | FQKWAAVVVP SGEEQRYTCH |
| | | | VQHEGLPEPV TLRWKP |

### REFERENCES

Barber & Arena de Souza et al., Structure-guided stabilization of pathogen-derived peptide-HLA-E complexes using non-natural amino acids conserves native TCR recognition, Eur J Immunol. 2022 April; 52(4): 618-632
Gadola et al. Structure of human CD1b with bound ligands at 2.3 Å, a maze for alkyl chains. Nat Immunol. 2002 August; 3(8):721-6. doi: 10.1038/ni821.

Kanevskiy et al., Dimorphism of HLA-E and Its Disease Association. Int J Mol Sci. 2019 November; 20(21): 5496
Kotsiou E, Brzostek J, Gould K G. Properties and applications of single-chain major histocompatibility complex class I molecules. Antioxid Redox Signal. 2011; 15(3):645-655. doi:10.1089/ars.2010.3694
Low et al., PLoS One. 2012; 7(12):e51397. doi: 10.1371/journal.pone.0051397
Mitaksov et al., Structural engineering of pMHC reagents for T cell vaccines and diagnostics, Chem Biol. 2007 August; 14(8):909-22
Sami, M (2007), Crystal structures of high affinity human T-cell receptors bound to peptide major histocompatibility complex reveal native diagonal binding geometry, Prot Eng Des Sel, 20:397-403
Sharpe et al., HLA-E: Exploiting pathogen-host interactions for vaccine development. Clin. Exp. Immunol. 2019; 196:167-177;
Truscott et al., Disulfide bond engineering to trap peptides in the MHC class I binding groove, Immunol. 2007 May 15; 178(10):6280-9
Yang et al., HLA-E Binding Peptide as a Potential Therapeutic Candidate for High-Risk Multiple Myeloma, Front. Oncol., 2021 v.11, Sec. Hematologic Malignancies

1.-90. (canceled)

91. An MHC class I molecule comprising or consisting of at least amino acids 1-276 of the amino acid sequence as set forth in SEQ ID NO: 1 or 2, or a variant of said MHC class I molecule having at least 90% sequence homology or identity thereto, such as at least 95% sequence homology or identity thereto, or such as at least 98% sequence homology or identity thereto,
with the proviso that the amino acids corresponding to positions
i) 4 and 102 of SEQ ID NO: 1 or 2, or
ii) 12 and 94 of SEQ ID NO: 1 or 2, or
iii) 23 and 37 of SEQ ID NO: 1 or 2, or
iv) 115 and 125 of SEQ ID NO: 1 or 2
have been mutated to cysteines and linked by a disulfide bridge, and wherein the MHC class I molecule has increased stability compared to a corresponding wild type MHC class I molecule of identical sequence except not comprising said cysteine mutations linked by a disulfide bridge.

92. The MHC class I molecule according to claim 91, wherein said MHC class I molecule comprises or consists of the amino acid sequence as set forth in SEQ ID NO: 3 or SEQ ID NO: 4, or comprises or consists of a variant of said MHC class I molecule having at least 90% sequence homology or identity thereto, such as at least 95% sequence homology or identity thereto, or such as at least 98% sequence homology or identity thereto,
with the proviso that the amino acids corresponding to positions 4 and 102 of SEQ ID NO: 3 or 4 are cysteines and linked by a disulfide bridge.

93. The MHC class I molecule according to claim 91, wherein said MHC class I molecule comprises or consists of the amino acid sequence as set forth in SEQ ID NO: 5 or SEQ ID NO: 6, or comprises or consists of a variant of said MHC class I molecule having at least 90% sequence homology or identity thereto, such as at least 95% sequence homology or identity thereto, or such as at least 98% sequence homology or identity thereto,
with the proviso that the amino acids corresponding to positions 12 and 94 of SEQ ID NO: 5 or 6 are cysteines and linked by a disulfide bridge.

94. The MHC class I molecule according to claim 91, wherein said MHC class I molecule comprises or consists of the amino acid sequence as set forth in SEQ ID NO: 7 or SEQ ID NO: 8, or comprises or consists of a variant of said MHC class I molecule having at least 90% sequence homology or identity thereto, such as at least 95% sequence homology or identity thereto, or such as at least 98% sequence homology or identity thereto,
with the proviso that the amino acids corresponding to positions 115 and 125 of SEQ ID NO: 7 or 8 are cysteines and linked by a disulfide bridge.

95. The MHC class I molecule according to claim 91, wherein said MHC class I molecule comprises or consists of the amino acid sequence as set forth in SEQ ID NO: 9 or SEQ ID NO: 10, or comprises or consists of a variant of said MHC class I molecule having at least 90% sequence homology or identity thereto, such as at least 95% sequence homology or identity thereto, or such as at least 98% sequence homology or identity thereto,
with the proviso that the amino acids corresponding to positions 23 and 37 of SEQ ID NO: 9 or 10 are cysteines and linked by a disulfide bridge.

96. The MHC class I molecule according to claim 91, wherein the MHC class I molecule is from an organism selected from the group consisting of human, gorilla, chimpanzee, rhesus macaque and rodent, such as mouse or rat.

97. An MHC class I multimer or an MHC class I/peptide multimer comprising at least two MHC class I molecules according to claim 91.

98. The MHC class I multimer or the MHC class I/peptide multimer according to claim 97, wherein the MHC class I multimer or MHC class I/peptide multimer comprises at least three MHC class I molecules.

99. The MHC class I multimer or the MHC class I/peptide multimer according to claim 97, further comprising one or more labels.

100. The MHC class I multimer or the MHC class I/peptide multimer according to claim 99, wherein the one or more labels comprise at least one fluorescent label.

101. The MHC class I multimer or the MHC class I/peptide multimer according to claim 99, wherein the one or more labels comprise at least one oligonucleotide label comprising or consisting of DNA, RNA, and/or artificial nucleotides such as PLA or LNA.

102. A nucleic acid encoding the MHC class I monomers according to claim 91.

103. A composition comprising one or more MHC class I molecules according to claim 91.

104. The composition according to claim 103 which is a pharmaceutical composition.

105. A composition comprising one or more MHC class I multimers and/or MHC class I/peptide multimers according to claim 97.

106. The composition according to claim 105 which is a pharmaceutical composition.

107. A method for isolation of one or more immune cells, the method comprising the steps of:
a) providing a sample comprising a population of immune cells,
b) providing one or more MHC class I molecules according to claim 91;
c) contacting said MHC class I molecules with said sample; and
d) isolating immune cells specific for said MHC class I molecules.

108. A method for isolation of one or more immune cells, the method comprising the steps of:
a) providing a sample comprising a population of immune cells;
b) providing one or more MHC class I multimers or MHC class I/peptide multimers according to claim 97;
c) contacting said MHC class I multimers or MHC class I/peptide multimers with said sample; and
d) isolating immune cells specific for said MHC class I multimers or MHC class I/peptide multimers.

109. A method for detecting an immune cell response, comprising the steps of:
a) providing a sample comprising a population of immune cells;
b) providing one or more MHC class I molecules according to claim 91;
c) contacting said MHC class I molecules with said sample; and
d) measuring a presence, frequency, number, activity and/or state of said immune cells,
thereby detecting said immune cell response.

110. A method for detecting an immune cell response, comprising the steps of:
a) providing a sample comprising a population of immune cells;
b) providing one or more MHC class I multimers or MHC class I/peptide multimers according to claim 97;
c) contacting said MHC class I multimers or MHC class I/peptide multimers with said sample; and
d) measuring a presence, frequency, number, activity and/or state of said immune cells,
thereby detecting said immune cell response.


SEQUENCE LISTING (17 sequences)

SEQ ID NO: 1 - protein, 276 aa, Homo sapiens
GSHSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDRRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWKP

SEQ ID NO: 2 - protein, 276 aa, Homo sapiens [sequence omitted]

SEQ ID NO: 3 - protein, 276 aa, Synthetic construct
GSHCLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCCLGPDRRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWKP

SEQ ID NO: 4 - protein, 276 aa, Synthetic construct
GSHCLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCCLGPDGRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWKP

SEQ ID NO: 5 - protein, 276 aa, Synthetic construct
GSHSLKYFHTSCSRPGRGEPRFISVGYVDDTQFVRFDNDAASPRMVPRAPWMEQEGSEYWDRETRSARDTAQIFRVNLRTLRGYYNQSEAGSHCLQWMHGCELGPDRRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWKP

SEQ ID NO: 6 - protein, 276 aa, Synthetic construct
GSHSLKYFHTSCSRPGRGEPRFISVGYVDDTQFVRFDNDAASPRMVPRAPWMEQEGSEYWDRETRSARDTAQIFRVNLRTLRGYYNQSEAGSHCLQWMHGCELGPDGRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWKP

SEQ ID NO: 7 - protein, 276 aa, Synthetic construct
GSHSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDRRFLRGYECFAYDGKDYLCLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWKP

SEQ ID NO: 8 - protein, 276 aa, Synthetic construct
GSHSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDGRFLRGYECFAYDGKDYLCLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWKP

SEQ ID NO: 9 - protein, 280 aa, Synthetic construct
GSHSLKYFHTSVSRPGRGEPRFCSVGYVDDTQFVRFCNDAASPRMVPRAPWMEQEGSEYWDRETRSARDTAQIFRVNLRTLRGYYNQSEAGSHTLQWMHGCELGPDRRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWKPGSGS

SEQ ID NO: 10 - protein, 276 aa, Synthetic construct
GSHSLKYFHTSVSRPGRGEPRFCSVGYVDDTQFVRFCNDAASPRMVPRAPWMEQEGSEYWDRETRSARDTAQIFRVNLRTLRGYYNQSEAGSHTLQWMHGCELGPDGRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWKP

SEQ ID NO: 11 - protein, 19 aa, Synthetic construct
GSGSLHHILDAQKMVWNHR

SEQ ID NO: 12 - protein, 9 aa, Synthetic construct
RLPAKAPLL

SEQ ID NO: 13 - protein, 9 aa, Synthetic construct
VMAPRTLIL

SEQ ID NO: 14 - protein, 9 aa, Synthetic construct
SLLMWITQC

SEQ ID NO: 15 - protein, 9 aa, Synthetic construct
ALIAPVHAV

SEQ ID NO: 16 - protein, 276 aa, Synthetic construct
GSHSLKYFHTSCSRPGRGEPRFISVGYVDDTQFVRFDNDAASPRMVPRAPWMEQEGSEYWDRETRSARDTAQIFRVNLRTLRGYYNQSEAGSHCLQWMHGCELGPDRRFLRGYECFAYDGKDYLCLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWKP

SEQ ID NO: 17 - protein, 276 aa, Synthetic construct
GSHSLKYFHTSCSRPGRGEPRFISVGYVDDTQFVRFDNDAASPRMVPRAPWMEQEGSEYWDRETRSARDTAQIFRVNLRTLRGYYNQSEAGSHCLQWMHGCELGPDGRFLRGYECFAYDGKDYLCLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWKP